(19) United States
(12) Patent Application Publication
(10) Pub. No.: US 2016/0083477 A1
(43) Pub. Date: Mar. 24, 2016

(54) Removal of Cancer Cells by Circulating Virus-Specific Cytotoxic T-Cells Using Cancer Cell Targeted MHC Class 1 Compromising Multi-Function Proteins

(71) Applicant: Roche Glycart AG, Schlieren (CH)

(72) Inventors: Christian Klein, Bonstetten (CH); Hendrik Knoetgen, Penzberg (DE); Martina Schmittnaegel, Tutzing (DE); Pablo Umana, Wollerau (CH)

(73) Assignee: Roche Glycart AG, Schlieren (CH)

(21) Appl. No.: 14/724,571

(22) Filed: May 28, 2015

Related U.S. Application Data
(63) Continuation of application No. PCT/EP2013/074759, filed on Nov. 26, 2013.

(30) Foreign Application Priority Data
Nov. 30, 2012 (EP) 12195094.3

(51) Int. Cl. C07K 16/30; C07K 16/28; C07K 14/74; C12N 7/00

(52) U.S. Cl. CPC C07K 16/3053; C12N 7/00; C07K 16/2863; C07K 16/30; C07K 14/70539; C07K 2317/622; C12N 2710/16133; C07K 2319/30; C07K 2319/74; C07K 2319/33; C07K 2317/52

(57) ABSTRACT

Herein is reported a multi-function protein, characterized in that it comprises exactly one antigen presenting domain, exactly one antibody Fc-region, and at least one antigen binding site, wherein the antigen presenting domain comprises in N- to C-terminal direction either (i) a β2-microglobulin, and (ii) the extracellular domains α1, α2, and α3 of a class I MHC molecule with a relative frequency of less than 1%, or (i) a T-cell response eliciting peptide, (ii) a β2-microglobulin, and (iii) the extracellular domains α1, α2, and α3 of a class I MHC molecule with a relative frequency of 1% or more, wherein the antigen binding site binds to a cancer cell surface antigen.

This application is a 35 USC 371 of PCT International Application No. PCT/EP2013/074759, filed on Nov. 26, 2013, currently pending, which is incorporated by reference herein in its entirety, and which claims priority to European Application No. EP12195094.3, filed Nov. 30, 2012, now expired.

The sequence listing includes the sequences identified as Seq ID Nos: 1-138. This file is named P31355-US_Sequence_Listing.txt, is 153,697 bytes in size, was created on Apr. 17, 2015, and is IBM PC/XT/AT: MS-windows compatible.

Herein is reported a multi-function protein comprising an antibody fragment and a MHC class I component and its use for removal of cancer cells by targeted attraction of circulating virus-specific cytotoxic T-cells.

### BACKGROUND OF THE INVENTION

Melanoma chondroitin sulfate proteoglycan (MCSP) is a large transmembrane proteoglycan that is expressed in the majority of melanoma cancers. MCSP is also expressed on other cancers, including glioblastomas, osteosarcomas, chondrosarcomas, some types of ALL and AML, and in basal cell carcinomas. It serves as an early cell surface melanoma progression marker and is involved in stimulating tumor cell proliferation, metastasis, migration, invasion, and angiogenesis (see e.g. Staub, E., et al., FEBS Lett. 527 (2002) 114-118; Campoli, M., et al., Crit. Rev. Immun. 24 (2004) 267-296; Vergilis, I. J., J. Invest. Dermatol. 125 (2005) 526-531; Yang, J., J. Chem. Biol. 165 (2004) 881-891; Luo, W., J. Immunol. 176 (2006) 6046-6054).

The MHC Class I protein consists of an α-chain (α-1 to 3 and a transmembrane domain) and β2-microglobulin. It is polygenic (3 gene loci for MHC-class I protein in the haploid genome) giving rise to six different MHC class I protein α-chains (in humans two HLA-A, two HLA-B, two HLA-C). The MHC is further polymorphic. The human HLA-A allele A*0201 is prevalent in about 30% to 50% of the caucasian population (see e.g. Player, M. A., et al., J Immunother. Emphasis Tumor Immunol. 19 (1996) 357-363).

Human cytomegalovirus huCMV (=human herpesvirus 5, HHV-5) is one of the largest human viruses. Its genome comprises around 230,000 bp linear double stranded DNA and encodes more than 160 proteins (see e.g. Davison, A. J., et al., J. Gen. Virol. 84 (2003) 17-28).

The CMV has evolved to become a sublime parasite of the human genome and it is a potent immunogen and triggers strong immune responses from all arms of the immune system. This virus appears to be among the most immunodominant antigens known to the human immune system and stimulates CD8⁺-T-cell responses of unprecedented magnitude.

The CMV “latency” depends on chronic immune suppression of CMV viruses rather than a change in the pattern of viral transcription (see e.g. Moss & Khan, Human Immunology 65 (2004) 456-464).

CD8⁺-T-cell immune responses are not directed evenly against all CMV proteins but are focused. The CMV proteins pp65 and IE-1 are the predominant targets (see e.g. McLaughlin-Taylor, E., et al., J. Med. Virol. 43 (1994) 103-110; Moss & Khan, Human Immunology 65 (2004) 456-464).

The frequency of CMV-specific T-cells is very high with frequencies for individual peptides in the order of up to 1 to 2% of the total CD8⁺-T-cell repertoire (see e.g. Moss & Khan, Human Immunology supra; Wills, M. R., et al., J. Virol. 70 (1996) 7569-7579).

The CMV-specific CD8⁺-T-cell response increases markedly with age and individual HLA-peptide tetramers frequently stain in excess of 10% of the total CD8⁺-T-cell pool (see e.g. Khan, N., et al., J. Immunol. 169 (2002) 1984-1992).

The total CD8⁺-T-cell response in healthy elderly donors could constitute approximately 50% of the CD8⁺-T-cell repertoire.

The enormous CD8⁺-T-cell expansions are often very clonally restricted, and it is estimated that CMV is the cause of at least 30% of the clonal CD8⁺-T-cell expansions that are seen in peripheral blood with aging. The total CD8⁺-T-cell count is twice as high in CMV-seropositive donors older than age 60 years in comparison to a CMV-seronegative cohort (see e.g. Looney, R. J., et al., Clin. Immunol. 90 (1999) 213-219).

A fusion of soluble HLA and β-2-microglobulin is reported by Mottez, E., et al., Eur. J. Immunol. 21 (1991) 467-471; Godeau, F., et al., J. Biol. Chem. 267 (1992) 24223-24229 and Mage, M. G., et al., Proc. Natl. Acad. Sci. 89 (1992) 10658-10662. A fusion of viral-derived peptide with soluble HLA and β-2-microglobulin is reported by Mottez, E., et al., J. Exp. Med. 181 (1995) 493-502. A fusion of an immunoglobulin heavy chain with soluble HLA and co-expressed β-2-microglobulin is reported by Dal Porto, J., et al., Proc. Natl. Acad. Sci. USA 90 (1993) 6671-6675. A tetrameric multi-function protein of biotinylated peptide-soluble HLA and β-2-microglobulin with streptavidin chemically coupled to a Fab is described by Robert, B., et al., Eur. J. Immun. 30 (2000) 3165-3170. A chemically coupled Fab with a fusion of viral-derived peptide with soluble HLA and β-2-microglobulin is reported by Robert, B., et al., Cancer Immunity 1 (2001) 2. A fusion of a viral-derived peptide with soluble HLA and β-2-microglobulin to a murine monoclonal antibody heavy chain is reported by Greten, T. F., et al., J. Immunol. Methods 271 (2002) 125-135. An E. coli expression of scFv fusions without peptide, in vitro refolding and peptide loading is reported by Lev, A., et al., J. Immunol. 169 (2002) 2988-2996; Lev, A., Proc. Natl. Acad. Sci. 101 (2004) 9051-9056, and Novak, H., et al., Int. J. Cancer 120 (2007) 329-336. The use of biotinylated soluble MHC loaded with peptides and coupled to streptavidin fused Fab or scFv antibodies is reported by Mous, R., et al., Leukemia 20 (2006) 1096-1102.

In WO 2005/099361 are reported MHC class I—peptide-antibody conjugates with modified beta-2-microglobulin. Exemplary conjugates as reported in WO 2005/099361 are obtained by in vitro conjugation of the alpha chain of the MHC-multi-function protein (HLA) or by the co-expression from separate genes in the same cell.

In US 2004/0091488 antigenic constructs of major histocompatibility multi-function protein class I antigens with specific carrier molecules are reported. These reported fusion polypeptides lack the hinge region.

In WO 02/102299 methods and pharmaceutical compositions for immune deception, particularly useful in the treatment of cancer are reported. Antibody-mediated targeting of human single-chain class I MHC with covalently linked peptides induces efficient killing of tumor cells by tumor or viral-specific cytotoxic T lymphocytes are reported by Oved, K., et al. (Immunotherapy 54 (2005) 867-879). Robert, B., et al. (Cancer Immun. 1 (2001) 1-13) report redirecting anti-viral CTL against cancer cells by surface targeting of monomeric MHC class 1-viral peptide conjugated to antibody fragments. Active antiviral T-lymphocyte response can be redirected against tumor cells by antitumor antibody×MHC/viral peptide conjugates (Cresson, V., et al., Clin. Cancer Res. 12 (2006) 7422-7430). Robert, B., et al. (Eur. J. Immunol. 30 (2000) 3165-3170) report antibody-conjugated MHC class I tetramers can target tumor cells for specific lysis by T lymphocytes. Antigenic constructs of major histocompatibility complex class I antigens with specific carrier molecules, the preparation and use thereof are reported in US 2004/0091488. Bluemel, C., et al. (Cancer Immunol. 59 (2010) 1197-1209) report epitope distance to the target cell membrane and antigen size determine the potency of T cell-mediated lysis by BiTE antibodies specific for a large melanoma surface antigen. Redirected lysis of human melanoma cells by a MCSP/CD3-bispecific BiTE antibody that engages patient derived T cells is reported by Torisu-Itakura, H., et al. (J. Immunother. 34 (2011) 597-605). Greten, T., et al. (J. Immunol. Meth. 271 (2002) 125-135) report peptide-beta2-microglobulin-MHC fusion molecules bind antigen-specific T cells and can be used for multivalent MHC-Ig complexes. Redirection of CMV-specific CTL towards B-CLL via CD20-targeted HLA/CMV complexes is reported by Mous, R., et al. (Leukemia 20 (2006) 1096-1102). In WO 2012/175508 removal of target cells by circulating virus-specific cytotoxic T-cells using MHC class I comprising complexes is reported.

### SUMMARY OF THE INVENTION

Herein is reported a multi-function protein comprising exactly one antigen presenting domain as first part, one antibody Fc-region as second part, and at least one antigen binding site that is derived from an antibody and that specifically binds to a target antigen as third part.

With the multi-function protein as reported herein existing virus-specific circulating cytotoxic T-cells (T-memory-cells and/or T-effector-cells) of an individual can be directed to cells expressing the target antigen, to which the antibody derived part of the multi-function protein specifically binds to. Thereafter by dressing these cells with a MHC class I complex an acute viral infection by the virus-derived peptide linked to the MHC class I protein multi-function protein is mimicked and cytotoxic cells are attracted resulting in the removal of the targeted cell.

One aspect as reported herein is a multi-function protein, characterized in that it comprises
exactly one antigen presenting domain,
exactly one antibody Fc-region, and
at least one antigen binding site,
wherein the antigen presenting domain comprises in N- to C-terminal direction
   either
      (i) a β2-microglobulin, and
   (ii) the extracellular domains α1, α2, and α3 of a class I MHC molecule with a relative frequency of less than 1%,
   or
      (i) the extracellular domains α1, α2, and α3 of a class I MHC molecule with a relative frequency of less than 1%, and
      (ii) a β2-microglobulin,
   or
      (i) a T-cell response eliciting peptide,
      (ii) a β2-microglobulin, and
      (iii) the extracellular domains α1, α2, and α3 of a class I MHC molecule with a relative frequency of 1% or more,
   or
      (i) a T-cell response eliciting peptide,
      (ii) the extracellular domains α1, α2, and α3 of a class I MHC molecule with a relative frequency of 1% or more, and
      (iii) a β2-microglobulin,
wherein the antigen binding site binds to a cancer cell surface antigen.

In one embodiment the multi-function protein is glycosylated.

In one embodiment the antigen presenting domain is a fusion polypeptide comprising in N- to C-terminal direction the listed components. In one embodiment the antigen presenting domain is recombinantly produced as a complete molecule.

In one embodiment the antibody Fc-region comprises a first and second disulfide-linked Fc-region polypeptide, whereby the antigen binding site comprises the first Fc-region polypeptide.

In one embodiment the antigen binding site comprises i) a (cognate) pair of an antibody heavy chain and an antibody light chain, whereby the individual chains can be wild-type chains or modified chains (substituted, mutated or domain exchanged), or ii) a scFv fusion polypeptide comprising in N- to C-terminal direction a scFv antibody fragment and an antibody Fc-region polypeptide, or iii) a scFab fusion polypeptide comprising in N- to C-terminal direction a scFab and an antibody Fc-region polypeptide.

In one embodiment the antibody light chain pairs only with its cognate heavy chain (i.e. the antibody light chain is no common light chain).

In one embodiment i) the antigen presenting domain is linked to the N-terminus of the heavy chain or to the N-terminus of the light chain of the antigen binding site, or ii) the antigen presenting domain is linked to the C-terminus of the heavy chain or to the C-terminus of the light chain of the antigen binding site, or iii) the antigen presenting domain is linked to the N- or C-terminus of the scFv fusion polypeptide, or iv) the antigen presenting domain is linked to the N- or C-terminus of the scFab fusion polypeptide, or iv) the antigen presenting domain is linked to the N- or C-terminus of the second Fc-region polypeptide.

In one embodiment the cancer cell surface antigen is melanoma-associated chondroitin sulfate proteoglycan (MCSP).

In one embodiment the multi-function protein is a covalent multi-function protein.

In one embodiment the T-cell response eliciting peptide is a virus-derived peptide. In one embodiment the T-cell response eliciting peptide is a CD8⁺-T-cell response eliciting peptide.

In one embodiment the virus is selected from adenovirus, human herpesvirus 1, human herpesvirus 2, human herpesvirus 4 (Epstein-Barr virus), hepatitis-B-virus, hepatitis-C-virus, human cytomegalovirus, human immunodeficiency virus, human papillomavirus type 16, human papillomavirus type 18, human papillomavirus type 31, human papillomavirus type 33, human papillomavirus type 35, human papillomavirus type 39, human papillomavirus type 45, human papillomavirus type 51, human papillomavirus type 52, human papillomavirus type 56, human papillomavirus type 58, human papillomavirus type 59, human papillomavirus type 68, human papillomavirus type 73, human papillomavirus type 82, human T-cell lymphotropic virus type I, human influenza A virus, human influenza B virus, vaccinia virus, dengue virus. In one embodiment the virus-derived peptide is selected from NLVPMVATV (SEQ ID NO: 01), VTEHDTLLY (SEQ ID NO: 02), NTDFRVLEL (SEQ ID NO: 03), CVETMCNEY (SEQ ID NO: 04), VLEETSVML (SEQ ID NO: 05), NLVPMVATV (SEQ ID NO: 06), RIFAELEGV (SEQ ID NO: 07), IIYTRNHEV (SEQ ID NO: 08), VLAELVKQI (SEQ ID NO: 09), AVGGAVASV (SEQ ID NO: 10), TVRSHCVSK (SEQ ID NO: 11), IMREFNSYK (SEQ ID NO: 12), GPISHGHVLK (SEQ ID NO: 13), ATVQGQNLK (SEQ ID NO: 14), VYALPLKML (SEQ ID NO: 15), AYAQKIFKIL (SEQ ID NO: 16), QYDPVAALF (SEQ ID NO: 17), YVKVYLESF (SEQ ID NO: 18), DIYRIFAEL (SEQ ID NO: 19), VFETSGGLVV (SEQ ID NO: 20), KARDHLAVL (SEQ ID NO: 21), QARLTVSGL (SEQ ID NO: 22), KARAKKDEL (SEQ ID NO: 23), QIKVRVDMV (SEQ ID NO: 24), RRRHRQDAL (SEQ ID NO: 25), ARVYEIKCR (SEQ ID NO: 26), KMQVIGDQY (SEQ ID NO: 27), NVRRSWEEL (SEQ ID NO: 28), CPSQEPMSIYVY (SEQ ID NO: 29), KPGKISHIMLDVA (SEQ ID NO: 30), ELRRKMMYM (SEQ ID NO: 31), IPSINVHHY (SEQ ID NO: 32), FEQPTETPP (SEQ ID NO: 33), YAYIYTTYL (SEQ ID NO: 34), QEFFWDANDIY (SEQ ID NO: 35), YEQHKITSY (SEQ ID NO: 36), QEPMSIYVY (SEQ ID NO: 37), SEHPTFTSQY (SEQ ID NO: 38), QAIRETVEL (SEQ ID NO: 39), TRATKMQVI (SEQ ID NO: 40), DALPGPCI (SEQ ID NO: 41), CEDVPSGKL (SEQ ID NO: 42), HERNGFTVL (SEQ ID NO: 43), PTFTSQYRIQGKL (SEQ ID NO: 44), QMWQARLTV (SEQ ID NO: 45), HELLVLVKKAQL (SEQ ID NO: 46), or DDYSNTHSTRYV (SEQ ID NO: 47), SLYNTVATL (SEQ ID NO: 48), GLCTLVAML (SEQ ID NO: 49), GILGFVFTL (SEQ ID NO: 50), STNRQSGRQ (SEQ ID NO: 51), LLFGYPVYV (SEQ ID NO: 52), FAEGFVRAL (SEQ ID NO: 53), LIVIGILIL (SEQ ID NO: 54), or ILHTPGCV (SEQ ID NO: 55), WYAQIQPHW (SEQ ID NO: 56), AFSGVSW™ (SEQ ID NO: 57), ILIGVVITW (SEQ ID NO: 58), MMIPTVVAF (SEQ ID NO: 59), PFPQSNAPI (SEQ ID NO: 60), LLLTLLATV (SEQ ID NO: 61), IVLEHGSCV (SEQ ID NO: 62), LLFKTENGV (SEQ ID NO: 63), PLNEAIMAV (SEQ ID NO: 64), NLVRLQSGV (SEQ ID NO: 65), LVISGLFPV (SEQ ID NO: 66), LLLVAHYAI (SEQ ID NO: 67), LALLAAFKV (SEQ ID NO: 68), VILAGPMPV (SEQ ID NO: 69), HVLGRLITV (SEQ ID NO: 70), or a variant thereof comprising of from 1 to 3 amino acid exchanges, additions, and/or deletions.

In one embodiment the virus-derived peptide is a human cytomegalovirus-derived peptide. In one embodiment the virus-derived peptide has an amino acid sequence selected from the group of SEQ ID NO: 01 to SEQ ID NO: 47. In one embodiment the virus-derived peptide has the amino acid sequence of SEQ ID NO: 01.

In one embodiment the class I MHC molecule with a relative frequency of 1% or more has a relative frequency of 10% or more.

In one embodiment the class I MHC molecule with a relative frequency of 1% or more is HLA-A*0201, or HLA-A*1101, or HLA-A*2402, or HLA-A*340101, or HLA-C*0304, or HLA-C*0401, or HLA-C*0702.

In one embodiment the class I MHC molecule with a relative frequency of 1% or more is selected depending on the region of the individual to whom the multi-function protein is to be administered as follows:
for an individual of European origin the class I MHC molecule is selected from the group comprising HLA-A*0101, HLA-A*0201, HLA-A*0301, HLA-B*0702, HLA-B*0801, HLA-B*4402, HLA-C*0401, HLA-C*0501, HLA-C*0701, and HLA-C*0702,
for an individual of Australian origin the class I MHC molecule is selected from the group comprising HLA-A*0201, HLA-A*1101, HLA-A*2402, HLA-A*340101, HLA-B*1301, HLA-B*1521, HLA-B*5601, HLA-B*5602, HLA-C*0102, HLA-C*0401, HLA-C*0403, and HLA-C*1502,
for an individual of North American origin the class I MHC molecule is selected from the group comprising HLA-A*0201, HLA-A*2402, HLA-C*0202, HLA-C*0304, HLA-C*0401, and HLA-C*0702, and
for an individual of South-East-Asian origin the class I MHC molecule is selected from the group comprising HLA-A*1101, HLA-A*2402, HLA-B*1504, HLA-C*0102, HLA-C*0304, HLA-C*0702, and HLA-C*0801.

In one embodiment the class I MHC molecule with a relative frequency of 1% or more is selected depending on the region of the individual to whom the multi-function protein is to be administered as follows:
for an individual of European origin the class I MHC molecule is HLA-A*0201,
for an individual of Australian origin the class I MHC molecule is selected from the group comprising HLA-A*2402, HLA-B*1301, HLA-C*0102, and HLA-C*0401,
for an individual of North American origin the class I MHC molecule is selected from the group comprising HLA-A*2402, and HLA-C*0304, and
for an individual of South-East-Asian origin the class I MHC molecule is HLA-A*2402.

In one embodiment the antigen presenting domain comprising in N- to C-terminal direction a β2-microglobulin and the extracellular domains α1, α2 and α3 of a class I MHC molecule with a relative frequency of less than 1% further comprises at its N-terminus a peptide binding to the MHC-peptide binding grove.

In one embodiment the class I MHC molecule with a relative frequency of less than 1% is selected from the group comprising HLA-B*4201, HLA-B*5901, HLA-B*6701, and HLA-B*7802.

In one embodiment the antigen presenting domain comprises/consists of
(i) a virus-derived peptide,
(ii) β2-microglobulin, and
(iii) the soluble HLA-A allele A*0201.

In one embodiment the β2-microglobulin is human β2-microglobulin.

In one embodiment the β2-microglobulin is wild-type human β2-microglobulin.

In one embodiment the β2-microglobulin is consisting of the amino acid sequence of SEQ ID NO: 71 or is a functional variant thereof comprising of from 1 to 10 amino acid exchanges, additions, and/or deletions.

In one embodiment the β2-microglobulin is human β2-microglobulin and the class I MHC molecule with a relative frequency of 10% or more is human HLA-A*0201.

In one embodiment the extracellular domain (α1, α2 and α3) of a class I MHC molecule is consisting of the amino acid sequence of SEQ ID NO: 72 or is a functional variant thereof comprising of from 1 to 10 amino acid exchanges, additions, and/or deletions.

In one embodiment the antigen presenting domain comprises in N- to C-terminal direction a β2-microglobulin and the extracellular domains α1, α2 and α3 of a class I MHC molecule that has a relative frequency of occurrence of less than 1%.

In one embodiment the virus-derived peptide is fused to the β2-microglobulin via a first linker peptide.

In one embodiment the virus-derived peptide is fused to the N-terminus of the β2-microglobulin.

In one embodiment the β2-microglobulin is fused to the extracellular domain α1 of a class I MHC molecule via a second linker peptide.

In one embodiment the extracellular domains α3 of a class I MHC molecule is fused to one of the disulfide-linked polypeptide chains via a third linker peptide.

In one embodiment the first, second, and third linker peptide is the same or different.

In one embodiment the first linker peptide, the second linker peptide, and the third linker peptide are selected independently from each other from the amino acid sequences GS (SEQ ID NO: 73), GGS (SEQ ID NO: 74), GSG (SEQ ID NO: 136), GGGS (SEQ ID NO: 75), GGGSGGGS (SEQ ID NO: 76), GGGSGGGSGGGS (SEQ ID NO: 77), GGGSGGGSGGGSGGGS (SEQ ID NO: 78), GGGSGGGSGGGSGGGSGGGS (SEQ ID NO: 79), GGGGS (SEQ ID NO: 80), GGGGSGGGGS (SEQ ID NO: 81), GGGGSGGGGSGGGGS (SEQ ID NO: 82), GGGGSGGGGSGGGGSGGGGS (SEQ ID NO: 83), and GGGGSGGGGSGGGGSGGGGSGGGGS (SEQ ID NO: 84).

In one embodiment of all aspects the first linker peptide comprises the amino acid sequence of SEQ ID NO: 82.

In one embodiment of all aspects the second linker peptide comprises the amino acid sequence of SEQ ID NO: 83.

In one embodiment of all aspects the third linker peptide comprises the amino acid sequence of SEQ ID NO: 73.

In one embodiment the antigen presenting domain comprises in N- to C-terminal direction
(i) a virus-derived peptide that has an amino acid sequence selected from the group comprising SEQ ID NO: 01 to SEQ ID NO: 70,
(ii) a first linker peptide that has an amino acid sequence selected from the group comprising SEQ ID NO: 77, 78, 79, 82, 83, and 84,
(iii) a β2-microglobulin that has an amino acid sequence of SEQ ID NO: 71,
(iv) a second linker peptide that has an amino acid sequence selected from the group comprising SEQ ID NO: 77, 78, 79, 82, 83, and 84,
(v) the extracellular domains α1, α2, and α3 of a class I MHC molecule that has an amino acid sequence of SEQ ID NO: 72, and
(vi) a third linker peptide that has an amino acid sequence selected from the group comprising SEQ ID NO: 73, 77, 78, 79, 82, 83, 84, and 136.

In one embodiment
the first linker peptide has the amino acid sequence of SEQ ID NO: 82, and/or
the second linker peptide has the amino acid sequence of SEQ ID NO: 83, and/or
the third linker peptide has the amino acid sequence of SEQ ID NO: 136.

In one embodiment the multi-function protein is characterized in that the antigen presenting domain comprises in N- to C-terminal direction
(i) a virus-derived peptide that has an amino acid sequence selected from the group comprising SEQ ID NO: 01 to SEQ ID NO: 70,
(ii) a first linker peptide that has an amino acid sequence selected from the group comprising SEQ ID NO: 77, 78, 79, 82, 83, and 84,
(iii) a β2-microglobulin that has an amino acid sequence of SEQ ID NO: 71 or is a functional variant thereof comprising of from 1 to 10 amino acid exchanges, additions, and/or deletions,
(iv) a second linker peptide that has an amino acid sequence selected from the group comprising SEQ ID NO: 77, 78, 79, 82, 83, and 84,
(v) the extracellular domains α1, α2 and α3 of a class I MHC molecule that has an amino acid sequence of SEQ ID NO: 72 or is a functional variant thereof comprising of from 1 to 10 amino acid exchanges, additions, and/or deletions, and
(vi) a third linker peptide that has an amino acid sequence selected from the group comprising SEQ ID NO: 73, 77, 78, 79, 82, 83, 84, and 136,

In one embodiment the antibody Fc-region is selected from an antibody Fc-region of a human antibody of the class IgG or the class IgE.

In one embodiment the antibody Fc-region is selected from an antibody Fc-region of a human antibody of the subclass IgG1, or IgG2, or IgG3, or IgG4.

In one embodiment the antibody Fc-region is of a human antibody of the subclass IgG1 or IgG2 and comprises at least one mutation in E233, L234, L235, G236, D265, D270, N297, E318, K320, K322, A327, P329, A330, and/or P331 (numbering according to the EU index of Kabat).

In one embodiment the antibody Fc-region is of a human antibody of the subclass IgG1 or the subclass IgG2 with the mutations L234A and L235A, and/or the mutations D265A and N297A, and/or contains the PVA236 mutation, and/or contains the mutation P329G.

In one embodiment the antibody Fc-region is of a human antibody of the subclass IgG1 with the mutations L234A and L235A, and/or P329G.

In one embodiment the antibody Fc-region is of a human antibody of the subclass IgG4 with the mutation S228P and/or L235E.

In one embodiment the first and second antibody Fc-region polypeptide is selected independently of each other from the group comprising SEQ ID NO: 87 to 101.

In one embodiment the antibody Fc-region comprises two Fc-region polypeptides with the amino acid sequence of SEQ ID NO: 94.

In one embodiment the antibody Fc-region comprises two Fc-region polypeptides with the amino acid sequence of SEQ ID NO: 100.

In one embodiment the antibody Fc-region comprises two Fc-region polypeptides with the amino acid sequence of SEQ ID NO: 101.

In one embodiment the antibody Fc-region comprises a first Fc-region polypeptide with the amino acid sequence of SEQ ID NO: 89 and a second Fc-region polypeptide with the amino acid sequence of SEQ ID NO: 90.

In one embodiment the antibody Fc-region comprises a first Fc-region polypeptide with the amino acid sequence of SEQ ID NO: 97 and a second Fc-region polypeptide with the amino acid sequence of SEQ ID NO: 98.

In one embodiment the antibody Fc-region comprises a first Fc-region polypeptide with the amino acid sequence of SEQ ID NO: 102 and a second Fc-region polypeptide with the amino acid sequence of SEQ ID NO: 103.

One aspect as reported herein is a multi-function protein, characterized in that it comprises
exactly one antigen presenting domain, which comprises in N- to C-terminal direction
   either
      (i) a β2-microglobulin, and
      (ii) the extracellular domains α1, α2, and α3 of a class I MHC molecule with a relative frequency of less than 1%,
   or
      (i) the extracellular domains α1, α2, and α3 of a class I MHC molecule with a relative frequency of less than 1%, and
      (ii) a β2-microglobulin,
   or
      (i) a T-cell response eliciting peptide,
      (ii) a β2-microglobulin, and
      (iii) the extracellular domains α1, α2, and α3 of a class I MHC molecule with a relative frequency of 1% or more,
   or
      (i) a T-cell response eliciting peptide,
      (ii) the extracellular domains α1, α2, and α3 of a class I MHC molecule with a relative frequency of 1% or more, and
      (iii) a β2-microglobulin,
exactly one antibody Fc-region, and
at least one antigen binding site, which specifically binds to melanoma-associated chondroitin sulfate proteoglycan (MCSP).

One aspect as reported herein is a multi-function protein, characterized in that it comprises
exactly one antigen presenting domain, which comprises in N- to C-terminal direction
   (i) a T-cell response eliciting peptide,
   (ii) a β2-microglobulin, and
   (iii) the extracellular domains α1, α2, and α3 of a class I MHC molecule with a relative frequency of 1% or more,
exactly one antibody Fc-region, and
at least one antigen binding site, which specifically binds to melanoma-associated chondroitin sulfate proteoglycan (MCSP).

One aspect as reported herein is a multi-function protein, characterized in that it comprises
exactly one antigen presenting domain, which comprises in N- to C-terminal direction
   (i) a T-cell response eliciting peptide of SEQ ID NO: 01,
   (ii) a β2-microglobulin of SEQ ID NO: 71, and
   (iii) the extracellular domains α1, α2, and α3 of a class I MHC molecule of SEQ ID NO: 72,
exactly one antibody Fc-region, and
at least one antigen binding site, which specifically binds to melanoma-associated chondroitin sulfate proteoglycan (MCSP).

One aspect as reported herein is a multi-function protein, characterized in that it comprises
exactly one antigen presenting domain, which comprises in N- to C-terminal direction
   (i) a T-cell response eliciting peptide of SEQ ID NO: 01,
   (ii) a β2-microglobulin of SEQ ID NO: 71, and
   (iii) the extracellular domains α1, α2, and α3 of a class I MHC molecule of SEQ ID NO: 72,
exactly one antibody Fc-region, which comprises two disulfide-linked Fc-region polypeptides, whereof the first disulfide-linked Fc-region polypeptide has the amino acid sequence of SEQ ID NO: 97 and the second disulfide-linked Fc-region polypeptide has the amino acid sequence of SEQ ID NO: 98, and
at least one antigen binding site, which specifically binds to melanoma-associated chondroitin sulfate proteoglycan (MCSP).

One aspect as reported herein is a multi-function protein, characterized in that it comprises
exactly one antigen presenting domain, which comprises in N- to C-terminal direction
   (i) a T-cell response eliciting peptide of SEQ ID NO: 01,
   (ii) a β2-microglobulin of SEQ ID NO: 71, and
   (iii) the extracellular domains α1, α2, and α3 of a class I MHC molecule of SEQ ID NO: 72,
exactly one antibody Fc-region, which comprises two disulfide-linked Fc-region polypeptides, whereof the first disulfide-linked Fc-region polypeptide has the amino acid sequence of SEQ ID NO: 102 and the second disulfide-linked Fc-region polypeptide has the amino acid sequence of SEQ ID NO: 103, and
at least one antigen binding site, which specifically binds to melanoma-associated chondroitin sulfate proteoglycan (MCSP).

One aspect as reported herein is a multi-function protein, characterized in that it comprises
exactly one antigen presenting domain, which comprises in N- to C-terminal direction
   (i) a T-cell response eliciting peptide of SEQ ID NO: 01,
   (ii) a β2-microglobulin of SEQ ID NO: 71, and
   (iii) the extracellular domains α1, α2, and α3 of a class I MHC molecule of SEQ ID NO: 72,
exactly one antibody Fc-region, which comprises two disulfide-linked Fc-region polypeptides, whereof the first disulfide-linked Fc-region polypeptide has the amino acid sequence of SEQ ID NO: 97 and the second disulfide-linked Fc-region polypeptide has the amino acid sequence of SEQ ID NO: 98, and
at least one antigen binding site, which comprises an antibody light chain variable domain comprising an amino acid sequence from the group of SEQ ID NO: 104 to 106 and an antibody heavy chain variable domain comprising an amino acid sequence from the group of SEQ ID NO: 108 to 110, which specifically binds to melanoma-associated chondroitin sulfate proteoglycan (MCSP).

One aspect as reported herein is a multi-function protein, characterized in that it comprises
exactly one antigen presenting domain, which comprises in N- to C-terminal direction
   (i) a T-cell response eliciting peptide of SEQ ID NO: 01,
   (ii) a β2-microglobulin of SEQ ID NO: 71, and
   (iii) the extracellular domains α1, α2, and α3 of a class I MHC molecule of SEQ ID NO: 72,
exactly one antibody Fc-region, which comprises two disulfide-linked Fc-region polypeptides, whereof the first disulfide-linked Fc-region polypeptide has the amino acid sequence of SEQ ID NO: 102 and the second disulfide-linked Fc-region polypeptide has the amino acid sequence of SEQ ID NO: 103, and
at least one antigen binding site, which specifically binds to melanoma-associated chondroitin sulfate proteoglycan (MCSP), and which comprises an antibody light chain with a variable domain comprising an amino acid sequence from the group of SEQ ID NO: 104 to 106 and an antibody heavy chain with a variable domain comprising an amino acid sequence from the group of SEQ ID NO: 108 to 110.

One aspect as reported herein is a multi-function protein, characterized in that it comprises
exactly one antigen presenting domain, which comprises in N- to C-terminal direction
   (i) a T-cell response eliciting peptide of SEQ ID NO: 01,
   (ii) a β2-microglobulin of SEQ ID NO: 71, and
   (iii) the extracellular domains α1, α2, and α3 of a class I MHC molecule of SEQ ID NO: 72,
exactly one antibody Fc-region, which comprises two disulfide-linked Fc-region polypeptides, whereof the first disulfide-linked Fc-region polypeptide has the amino acid sequence of SEQ ID NO: 97 and the second disulfide-linked Fc-region polypeptide has the amino acid sequence of SEQ ID NO: 98, and
two antigen binding sites, which specifically bind to melanoma-associated chondroitin sulfate proteoglycan (MCSP), and which each comprise an antibody light chain with a variable domain comprising an amino acid sequence from the group of SEQ ID NO: 105 to 107 and an antibody heavy chain with a variable domain comprising an amino acid sequence from the group of SEQ ID NO: 110 to 112.

One aspect as reported herein is a multi-function protein, characterized in that it comprises
exactly one antigen presenting domain, which comprises in N- to C-terminal direction
   (i) a T-cell response eliciting peptide of SEQ ID NO: 01,
   (ii) a β2-microglobulin of SEQ ID NO: 71, and
   (iii) the extracellular domains α1, α2, and α3 of a class I MHC molecule of SEQ ID NO: 72,
exactly one antibody Fc-region, which comprises two disulfide-linked Fc-region polypeptides, whereof the first disulfide-linked Fc-region polypeptide has the amino acid sequence of SEQ ID NO: 102 and the second disulfide-linked Fc-region polypeptide has the amino acid sequence of SEQ ID NO: 103, and
at least one antigen binding site, which specifically binds to melanoma-associated chondroitin sulfate proteoglycan (MCSP), and which comprises an antibody light chain with a variable domain comprising an amino acid sequence from the group of SEQ ID NO: 105 to 107 and an antibody heavy chain with a variable domain comprising an amino acid sequence from the group of SEQ ID NO: 110 to 112, whereby the Fc-region of the antibody heavy chain is one of the disulfide-linked Fc-region polypeptides.

One aspect as reported herein is a multi-function protein, characterized in that it comprises
exactly one antigen presenting domain, which comprises in N- to C-terminal direction
   (i) a T-cell response eliciting peptide of SEQ ID NO: 01,
   (ii) a β2-microglobulin of SEQ ID NO: 71, and
   (iii) the extracellular domains α1, α2, and α3 of a class I MHC molecule of SEQ ID NO: 72,
exactly one antibody Fc-region, which comprises two disulfide-linked Fc-region polypeptides, whereof the first disulfide-linked Fc-region polypeptide has the amino acid sequence of SEQ ID NO: 97 and the second disulfide-linked Fc-region polypeptide has the amino acid sequence of SEQ ID NO: 98, and
at least one antigen binding site, which specifically binds to melanoma-associated chondroitin sulfate proteoglycan (MCSP), and which comprises an antibody light chain with a variable domain comprising an amino acid sequence from the group of SEQ ID NO: 104 to 106 and an antibody heavy chain with a variable domain comprising an amino acid sequence from the group of SEQ ID NO: 108 to 110, whereby the Fc-region of the antibody heavy chain is one of the disulfide-linked Fc-region polypeptides,
wherein the antigen presenting domain is linked to the N-terminus of one of the variable domains.

One aspect as reported herein is a multi-function protein, characterized in that it comprises
exactly one antigen presenting domain, which comprises in N- to C-terminal direction
   (i) a T-cell response eliciting peptide of SEQ ID NO: 01,
   (ii) a β2-microglobulin of SEQ ID NO: 71, and
   (iii) the extracellular domains α1, α2, and α3 of a class I MHC molecule of SEQ ID NO: 72,
exactly one antibody Fc-region, which comprises two disulfide-linked Fc-region polypeptides, whereof the first disulfide-linked Fc-region polypeptide has the amino acid sequence of SEQ ID NO: 102 and the second disulfide-linked Fc-region polypeptide has the amino acid sequence of SEQ ID NO: 103, and
two antigen binding site, which specifically bind to melanoma-associated chondroitin sulfate proteoglycan (MCSP), and which each comprise an antibody light chain with a variable domain comprising an amino acid sequence from the group of SEQ ID NO: 104 to 106 and an antibody heavy chain with a variable domain comprising an amino acid sequence from the group of SEQ ID NO: 108 to 110, whereby the Fc-regions of the antibody heavy chains are the disulfide-linked Fc-region polypeptides,
wherein the antigen presenting domain is linked to the N-terminus of one of the variable domains.

One aspect as reported herein is a multi-function protein, characterized in that it comprises
exactly one antigen presenting domain, which comprises in N- to C-terminal direction
   (i) a T-cell response eliciting peptide of SEQ ID NO: 01,
   (ii) a β2-microglobulin of SEQ ID NO: 71, and
   (iii) the extracellular domains α1, α2, and α3 of a class I MHC
exactly one antibody Fc-region, which comprises two disulfide-linked Fc-region polypeptides, whereof the first disulfide-linked Fc-region polypeptide has the amino acid sequence of SEQ ID NO: 97 and the second disulfide-linked Fc-region polypeptide has the amino acid sequence of SEQ ID NO: 98, and
two antigen binding site, which specifically bind to melanoma-associated chondroitin sulfate proteoglycan (MCSP), and which each comprise an antibody light chain with a variable domain comprising an amino acid sequence from the group of SEQ ID NO: 105 to 107 and an antibody heavy chain with a variable domain comprising an amino acid sequence from the group of SEQ ID NO: 110 to 112, whereby the Fc-regions of the antibody heavy chains are the disulfide-linked Fc-region polypeptides, wherein the antigen presenting domain is linked to the N-terminus of one of the heavy chain variable domains.

One aspect as reported herein is a multi-function protein, characterized in that it comprises
exactly one antigen presenting domain, which comprises in N- to C-terminal direction
   (i) a T-cell response eliciting peptide of SEQ ID NO: 01,
   (ii) a β2-microglobulin of SEQ ID NO: 71, and
   (iii) the extracellular domains α1, α2, and α3 of a class I MHC molecule of SEQ ID NO: 72,
exactly one antibody Fc-region, which comprises two disulfide-linked Fc-region polypeptides, whereof the first disulfide-linked Fc-region polypeptide has the amino acid sequence of SEQ ID NO: 102 and the second disulfide-linked Fc-region polypeptide has the amino acid sequence of SEQ ID NO: 103, and
two antigen binding site, which specifically bind to melanoma-associated chondroitin sulfate proteoglycan (MCSP), and which each comprise an antibody light chain with a variable domain comprising an amino acid sequence from the group of SEQ ID NO: 105 to 107 and an antibody heavy chain with a variable domain comprising an amino acid sequence from the group of SEQ ID NO: 110 to 112, whereby the Fc-regions of the antibody heavy chains are the disulfide-linked Fc-region polypeptides,
wherein the antigen presenting domain is linked to the N-terminus of one of the heavy chain variable domains.

One aspect as reported herein is a multi-function protein, characterized in that it comprises
one polypeptide chain of SEQ ID NO: 117,
one polypeptide chain of SEQ ID NO: 118,
two polypeptide chains each of SEQ ID NO: 119.

One aspect as reported herein is a multi-function protein, characterized in that it comprises
one polypeptide chain of SEQ ID NO: 137,
one polypeptide chain of SEQ ID NO: 118,
two polypeptide chains each of SEQ ID NO: 119.

In one embodiment the MCSP binding site comprises an antibody heavy chain variable domain comprising an HVR-H1 comprising the amino acid sequence of SEQ ID NO: 108, an HVR-H2 comprising the amino acid sequence of SEQ ID NO: 109, and an HVR-H3 comprising the amino acid sequence of SEQ ID NO: 110.

In one embodiment the MCSP binding site comprises an antibody light chain variable domain comprising an HVR-L1 comprising the amino acid sequence of SEQ ID NO: 104; an HVR-L2 comprising the amino acid sequence of SEQ ID NO: 105; and an HVR-L3 comprising the amino acid sequence of SEQ ID NO: 106.

In one embodiment the MCSP binding site comprises an antibody heavy chain variable domain comprising an HVR-H1 comprising the amino acid sequence of SEQ ID NO: 108; an HVR-H2 comprising the amino acid sequence of SEQ ID NO: 109; an HVR-H3 comprising the amino acid sequence of SEQ ID NO: 110; and an antibody light chain variable domain comprising an HVR-L1 comprising the amino acid sequence of SEQ ID NO: 104; an HVR-L2 comprising the amino acid sequence of SEQ ID NO: 105; and an HVR-L3 comprising the amino acid sequence of SEQ ID NO: 106.

In one embodiment the MCSP binding site comprises an antibody heavy chain variable domain comprising an HVR-H1 comprising the amino acid sequence of SEQ ID NO: 111, an HVR-H2 comprising the amino acid sequence of SEQ ID NO: 112, and an HVR-H3 comprising the amino acid sequence of SEQ ID NO: 110.

In one embodiment the MCSP binding site comprises an antibody light chain variable domain comprising an HVR-L1 comprising the amino acid sequence of SEQ ID NO: 107; an HVR-L2 comprising the amino acid sequence of SEQ ID NO: 105; and an HVR-L3 comprising the amino acid sequence of SEQ ID NO: 106.

In one embodiment the MCSP binding site comprises an antibody heavy chain variable domain comprising an HVR-H1 comprising the amino acid sequence of SEQ ID NO: 111; an HVR-H2 comprising the amino acid sequence of SEQ ID NO: 112; an HVR-H3 comprising the amino acid sequence of SEQ ID NO: 110; and an antibody light chain variable domain comprising an HVR-L1 comprising the amino acid sequence of SEQ ID NO: 107; an HVR-L2 comprising the amino acid sequence of SEQ ID NO: 105; and an HVR-L3 comprising the amino acid sequence of SEQ ID NO: 106.

In one embodiment the MCSP binding site comprises an antibody heavy chain variable domain having at least 95% sequence identity to the amino acid sequence of SEQ ID NO: 114; and an antibody light chain variable domain having at least 95% sequence identity to the amino acid sequence of SEQ ID NO: 113.

In one embodiment the MCSP binding site comprises an antibody heavy chain variable domain of SEQ ID NO: 114 and an antibody light chain variable domain of SEQ ID NO: 113.

In one embodiment the MCSP binding site comprises SEQ ID NO: 114 and SEQ ID NO: 113.

In one embodiment the MCSP binding site comprises an antibody heavy chain variable domain having at least 95% sequence identity to the amino acid sequence of SEQ ID NO: 116; and an antibody light chain variable domain having at least 95% sequence identity to the amino acid sequence of SEQ ID NO: 115.

In one embodiment the MCSP binding site comprises an antibody heavy chain variable domain of SEQ ID NO: 116; and an antibody light chain variable domain of SEQ ID NO: 115.

In one embodiment the MCSP binding site comprises SEQ ID NO: 116 and SEQ ID NO: 115.

In one aspect, the invention provides multi-function proteins comprising the binding specificity, i.e. HVRs or variable domains, of isolated antibodies that bind to MCSP. In particular, the anti-MCSP antibody binding specificity, i.e. HVRs or variable domains, comprised in the multi-function proteins as provided herein bind to a membrane proximal epitope of human MCSP. As discussed in Staub, E., et al. (FEBS Lett. 527 (2002) 114-118), the membrane proximal region of MCSP is comprised of multiple novel repeated domains, referred to as CSPG repeat domains.

One aspect as reported herein is a nucleic acid encoding the multi-function protein as reported herein.

In one embodiment the nucleic acid comprises two to four expression cassettes comprising structural genes encoding polypeptides with different amino acid sequence.

One aspect as reported herein is a host cell comprising the nucleic acid as reported herein.

One aspect as reported herein is a method of producing a multi-function protein as reported herein comprising culturing the host cell as reported herein so that the multi-function protein is produced.

In one embodiment the multi-function protein is recovered from the cells or the cultivation medium and thereby the multi-function protein is produced.

One aspect as reported herein is a pharmaceutical formulation comprising the multi-function protein as reported herein and optionally a pharmaceutically acceptable carrier.

In one embodiment the pharmaceutical formulation further comprises an additional therapeutic agent.

One aspect as reported herein is the multi-function protein as reported herein for use as a medicament.

One aspect as reported herein is the multi-function protein as reported herein for use in treating cancer.

One aspect as reported herein is the multi-function protein as reported herein for use in attracting virus-specific cytotoxic T-cells of an individual to a target.

One aspect as reported herein is the multi-function protein as reported herein for use in removal cancer cells.

One aspect as reported herein is the use of the multi-function protein as reported herein in the manufacture of a medicament.

In one embodiment the medicament is for treatment of cancer.

In one embodiment the medicament is for attracting virus-specific cytotoxic T-cells of an individual to a target.

In one embodiment the medicament is for removal cancer cells.

One aspect as reported herein is a method of treating an individual having cancer comprising administering to the individual an effective amount of the multi-function protein as reported herein.

In one embodiment the method further comprises administering an additional therapeutic agent to the individual.

One aspect as reported herein is a method of attracting virus-specific cytotoxic T-cells of an individual to a target in an individual comprising administering to the individual an effective amount of the multi-function protein as reported herein to attract virus-specific cytotoxic T-cells of an individual to a target.

One aspect as reported herein is a method of removal cancer cells in an individual comprising administering to the individual an effective amount of the multi-function protein as reported herein to remove/disintegrate cancer cells.

One aspect as reported herein is a method for the recombinant production of a multi-function protein comprising i) a fusion polypeptide of β2-microglobulin and the extracellular domains α1, α2 and α3 of a class I MHC molecule, ii) a pair of disulfide-linked polypeptide chains each comprising an antibody hinge region, and iii) at least one pair of an antibody light chain variable domain and an antibody heavy chain variable domain in a eukaryotic cell, comprising the steps of i) cultivating a eukaryotic cell comprising one or more nucleic acids encoding the multi-function protein, and ii) recovering the multi-function protein from the cell or the cultivation medium, wherein the multi-function protein comprises exactly one fusion polypeptide of β2-microglobulin and the extracellular domains α1, α2 and α3 of a class I MHC molecule.

In one embodiment the multi-function protein comprises exactly one MHC-derived polypeptide or exactly one fusion polypeptide comprising an MHC-derived molecule.

In one embodiment the multi-function protein is obtained with a concentration of 1 mg/ml or more in the cultivation medium. In one embodiment the multi-function protein is obtained with a concentration of 4 mg/ml or more in the cultivation medium.

In one embodiment the eukaryotic cell is a mammalian cell. In one embodiment the mammalian cell is a human embryonic kidney cell, or a chinese hamster ovary cell, or a baby hamster kidney cell, or a mouse myeloma cell.

The following embodiments can be combined with any of the aspects as reported herein. Also any embodiment as reported herein can be combined with any other embodiment or combination of embodiments as reported herein.

### DETAILED DESCRIPTION OF THE INVENTION

### Short Description of the Figures

FIG. 1 Annotated scheme of an exemplary multi-function protein as reported herein.

FIG. 2 Exemplary polypeptides comprised in the multi-function protein as reported herein: fusion polypeptides were N-terminally fused to either an antibody light chain or to an antibody heavy chain hinge region comprising polypeptide.

FIG. 3 Western blot of a SDS polyacrylamide gel of cell culture supernatant of HEK 293 cells transfected with the corresponding expression plasmids. Staining was performed with peroxidase conjugated polyclonal rabbit anti-human κ-light chain antibody and polyclonal rabbit anti-human IgG antibody conjugated to horseradish peroxidase.
Lanes: 1: two-armed peptide-β2-microglobulin-HLA-A0201-IgG-Fc; 2: one-armed peptide-β2-microglobulin-HLA-A0201-IgG-Fc+IgG-Fc; 3: one-armed peptide-β2-microglobulin-HLA-A0201-IgG-heavy chain+IgG-light chain+IgG-Fc; 4: one-armed peptide-β2-microglobulin-HLA-A0201-IgG-heavy chain+IgG-heavy chain+IgG-light chain; 5: two-armed β2-microglobulin-HLA-A0201-IgG-light chain+IgG-heavy chain; 6: two-armed peptide-β2-microglobulin-HLA-A0201-IgG-light chain+IgG-heavy chain; 7: two-armed peptide-β2-microglobulin-HLA-A0201-IgG-heavy chain+IgG-light chain; 8: two-armed peptide-β2-microglobulin-HLA-A0201-IgG-Fc-scFv; 9: one-armed peptide-β2-microglobulin-HLA-A0201-IgG-Fc+ one-armed IgG (heavy and light chain); 10: molecular weight marker; 11: reference standard IgG1 antibody.

FIGS. 4A-B Flow cytometric analysis to determine the number of CMV-specific cytolytic T-cells from different donors before and after in vitro stimulation with specific peptide: Analysis of 4 human donor derived peripheral blood lymphocytes (PBLs); anti-CD8 antibody conjugated to FITC label staining (BD, Cat. No. 345772) combined with ProS pentamer APC (Prolmmune, Cat. No. F008-4A-E) stained TCR recognizing MHC-class I (HLA-A*0201) loaded with CMV-derived peptide (NLVPMVATV, SEQ ID NO: 01); circle: CMV-specific CD8⁺-T-cells; FIG. 4A: Donor 1; FIG. 4B: Donor 3.

FIGS. 5A-C FIG. 5A: SDS-PAGE gel with Coomassie staining: lane 1: molecular weight standard, lane 2: one-armed peptide-β2-microglobulin-HLA-A0201-IgG-Fc+ one-armed IgG (heavy and light chain), non-reducing conditions; lane 3: one-armed peptide-β2-microglobulin-HLA-A0201-IgG-Fc+ one-armed IgG multi-function protein (heavy and light chain), reducing conditions.

FIG. 5B: Size exclusion chromatography chromatogram; 1: high molecular weight forms (0.7 area %); 2: monomeric multi-function protein (99.3 area %).

FIG. 5C: schematic molecule.

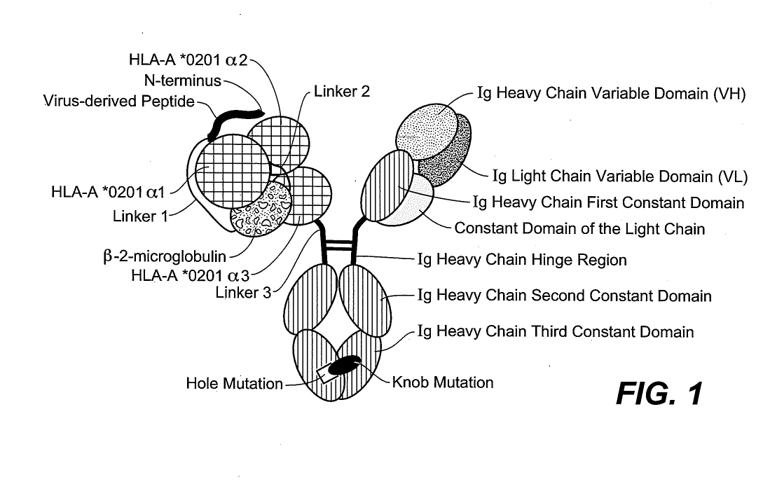

FIGS. 6A-C FIG. 6A-1) SDS-PAGE gel with Coomassie staining after protein A HPLC and SEC; non-reducing conditions; lane 1: molecular weight standard, lane 2: peptide-β2-microglobulin-HLA-A0201-HC+LC+IgG-Fc, lane 3: peptide-β2-microglobulin-HLA-A0201-HC+LC+ one-armed IgG (heavy and light chain).

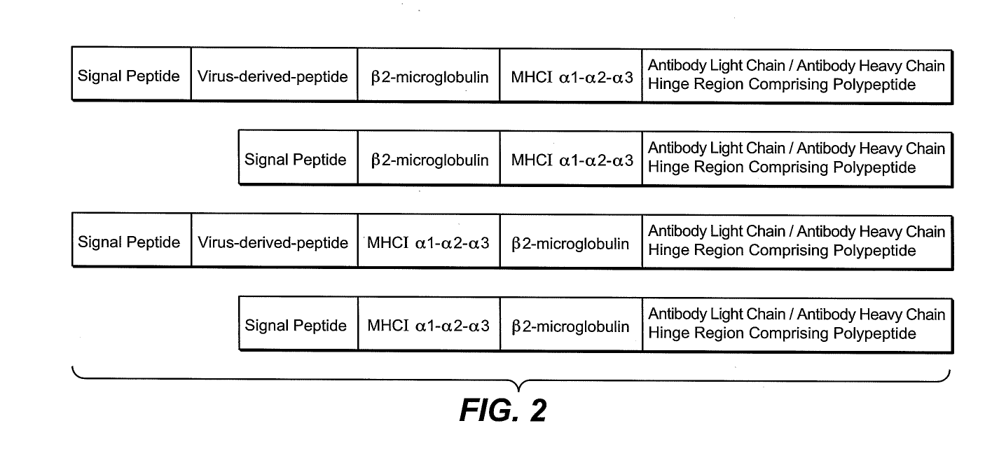

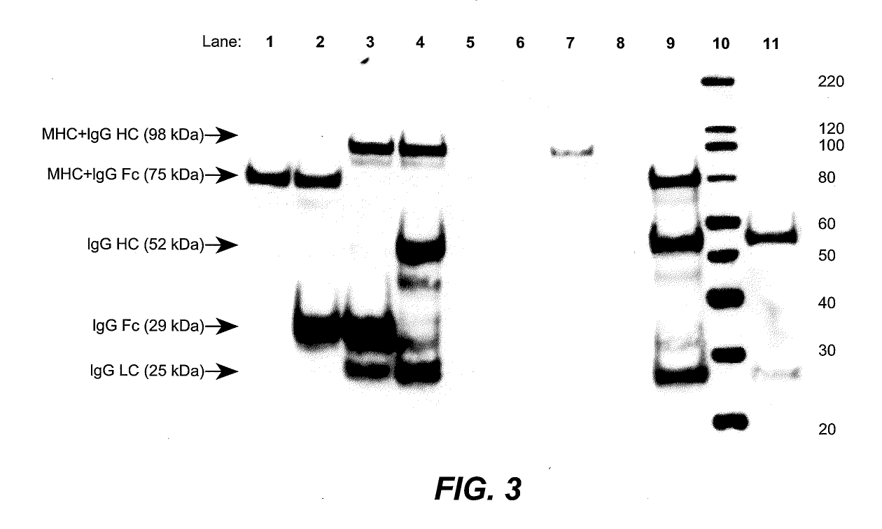

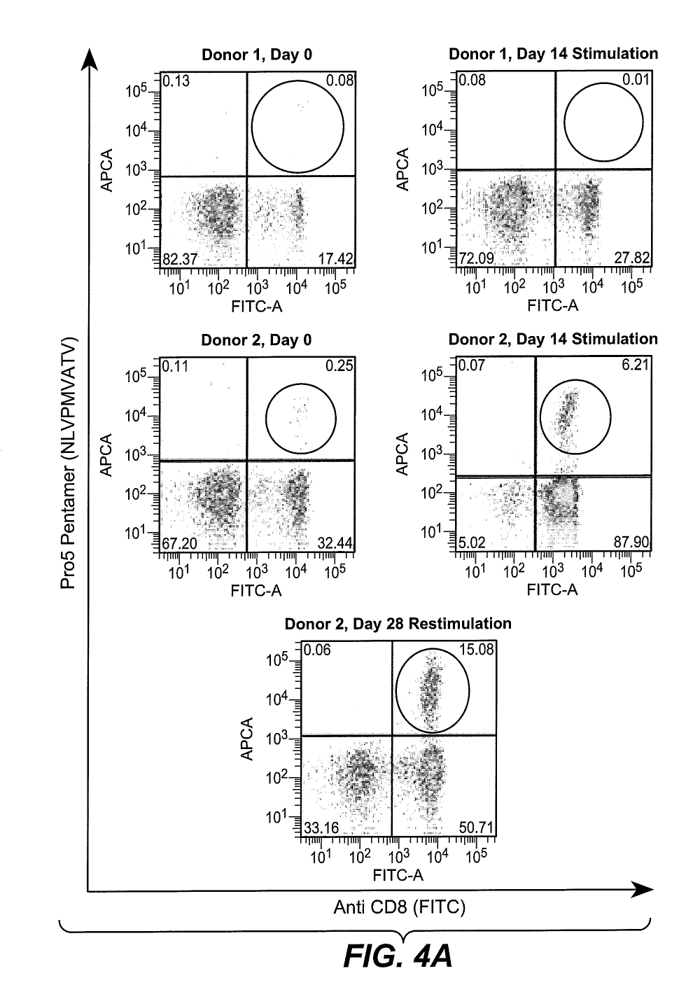

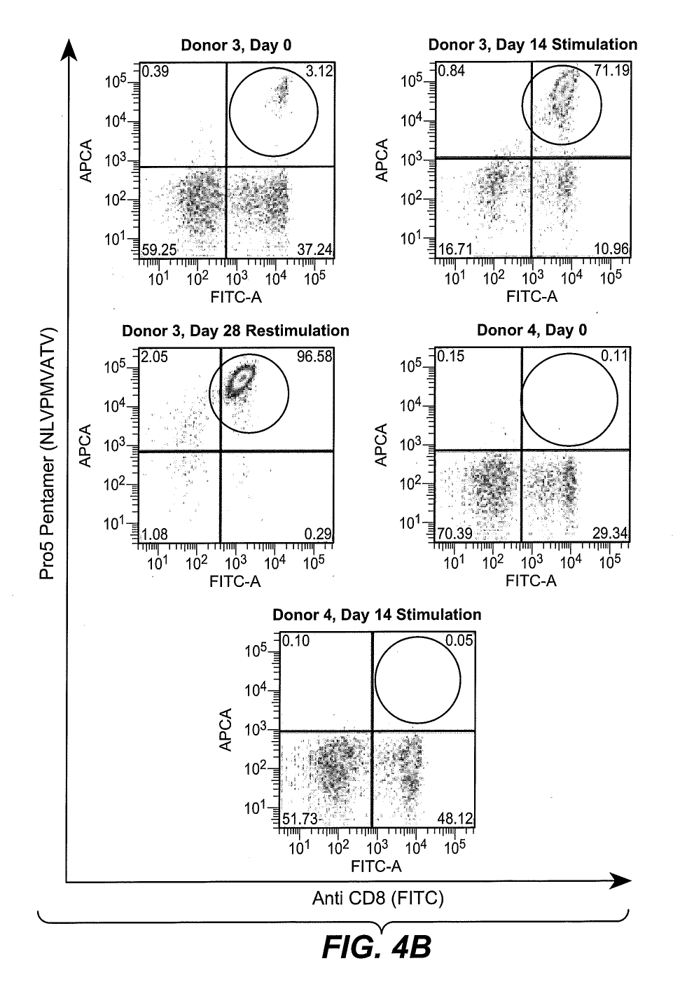

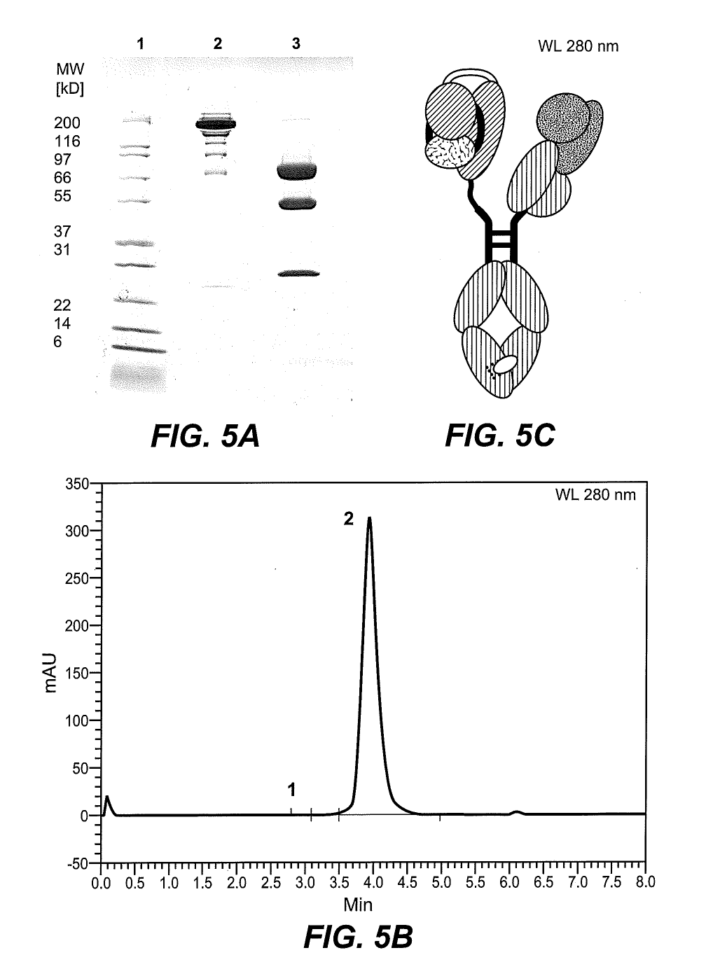

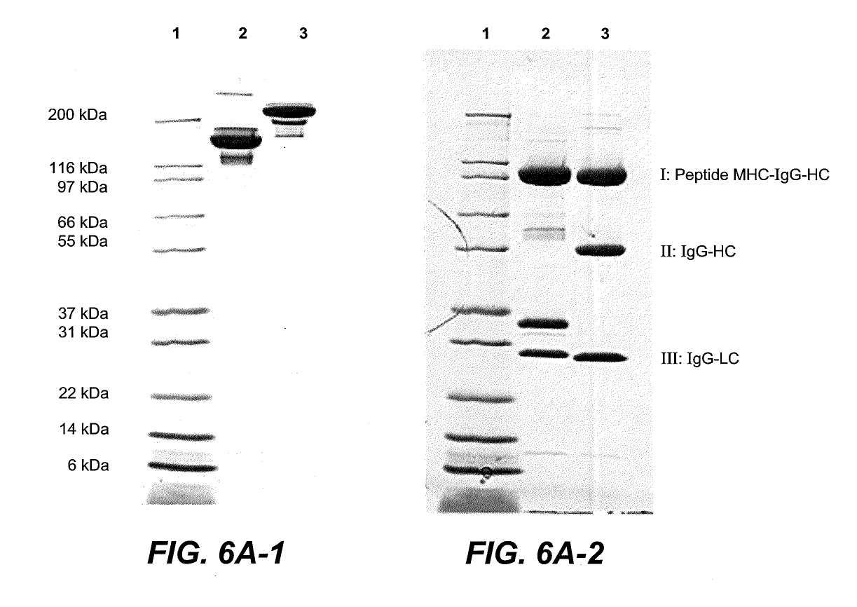

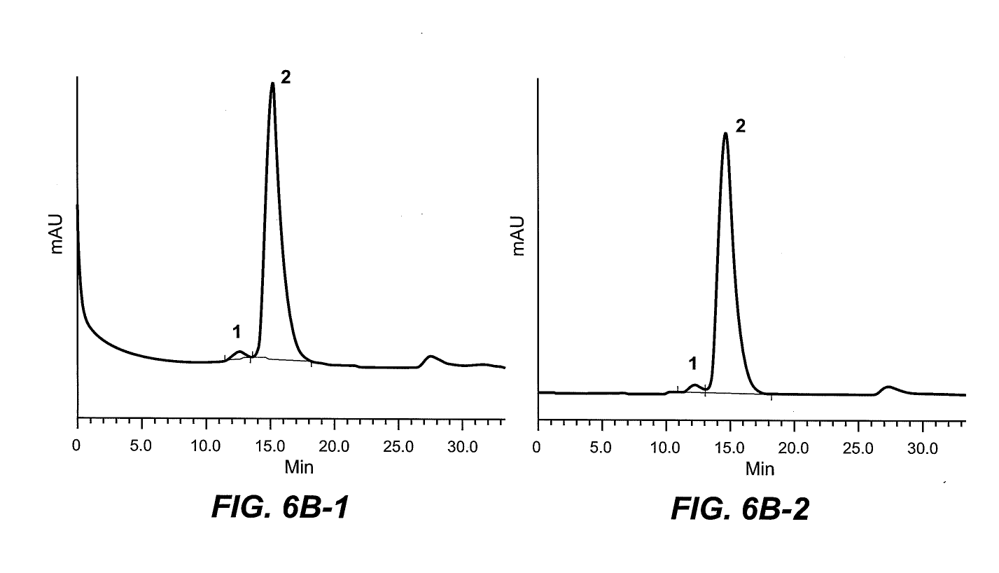

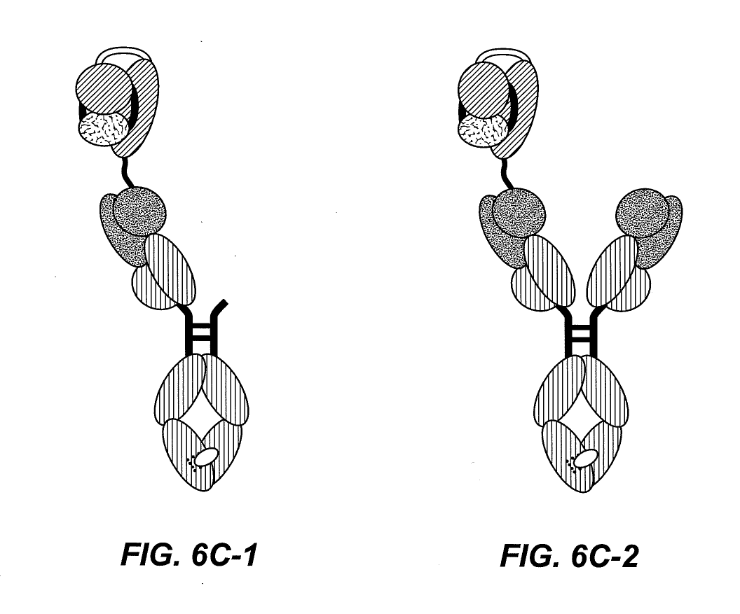

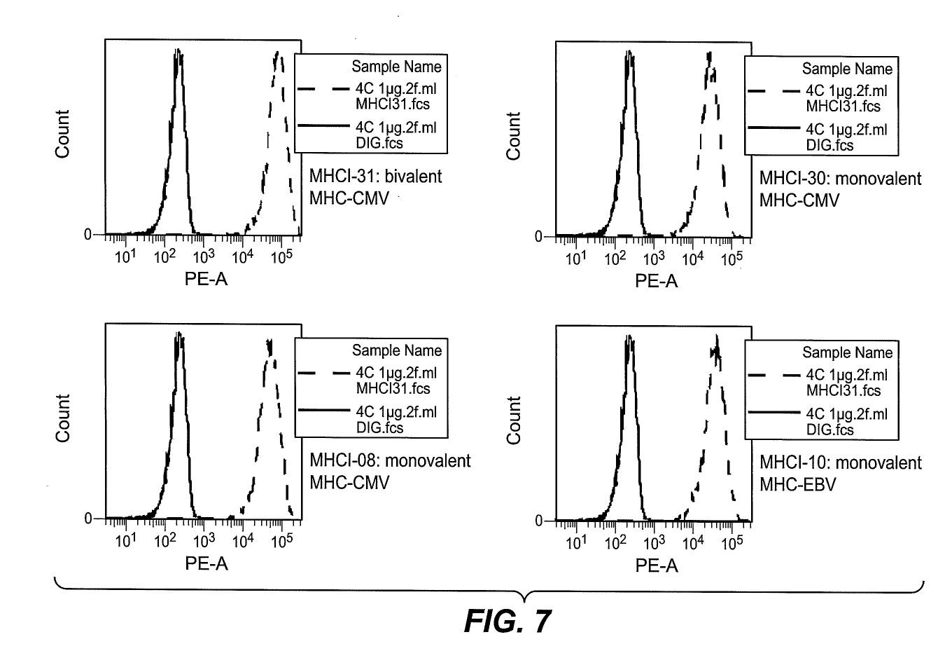

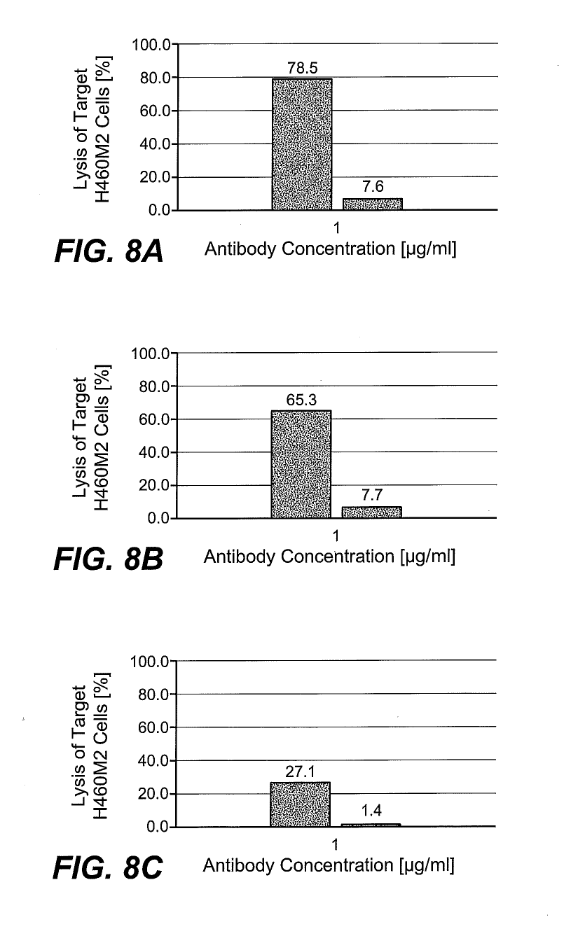

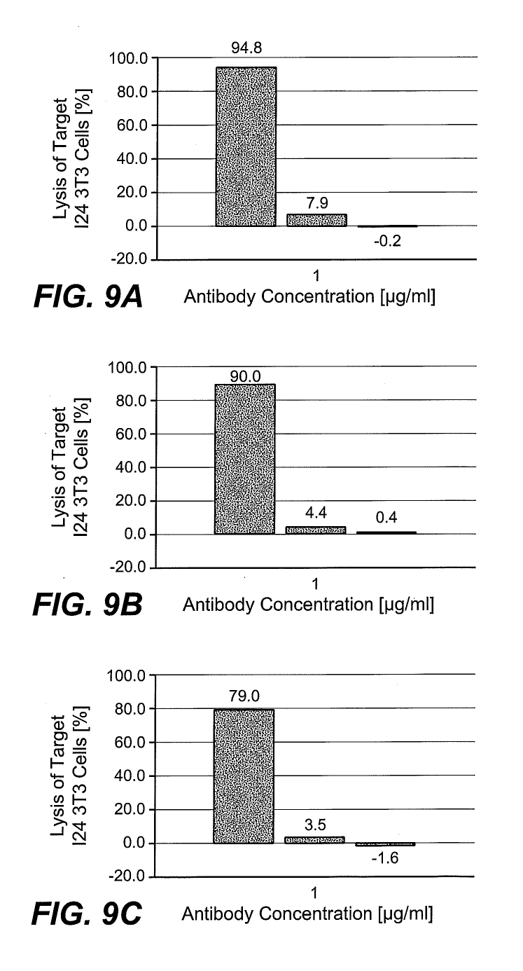

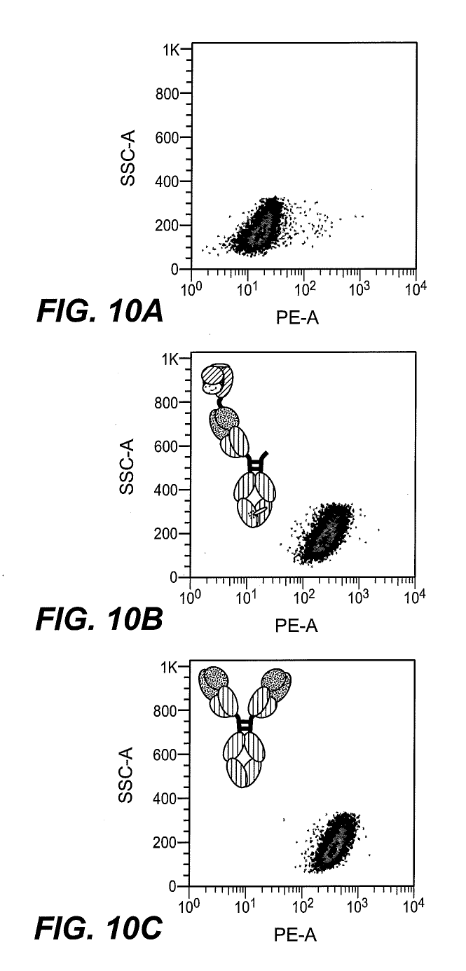

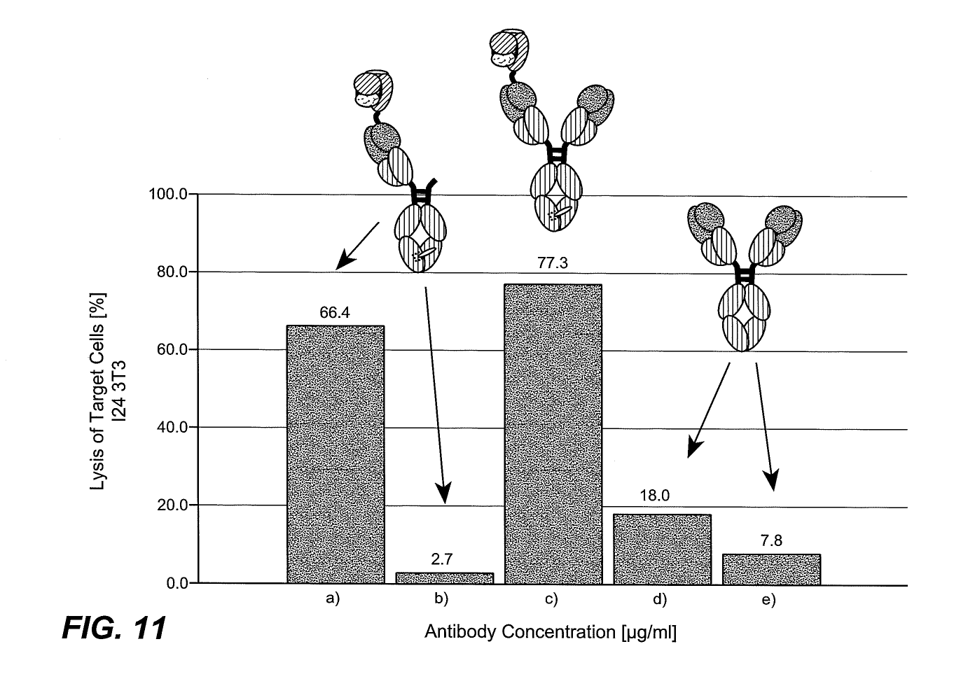

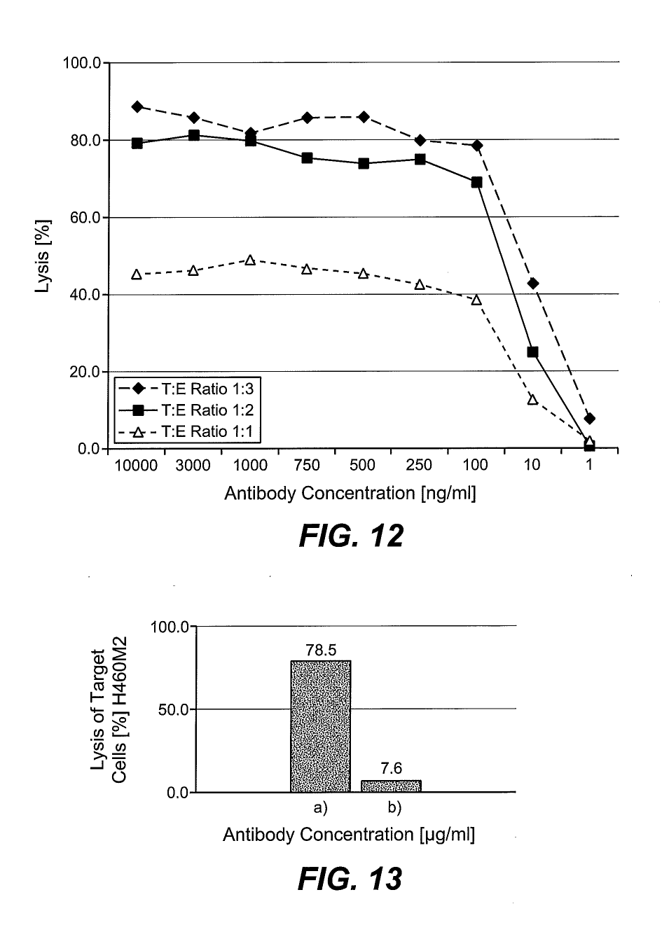

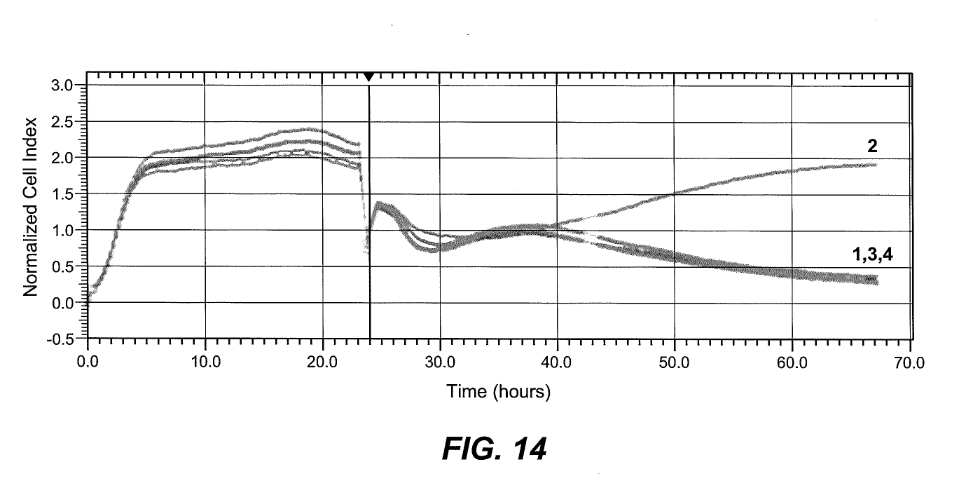

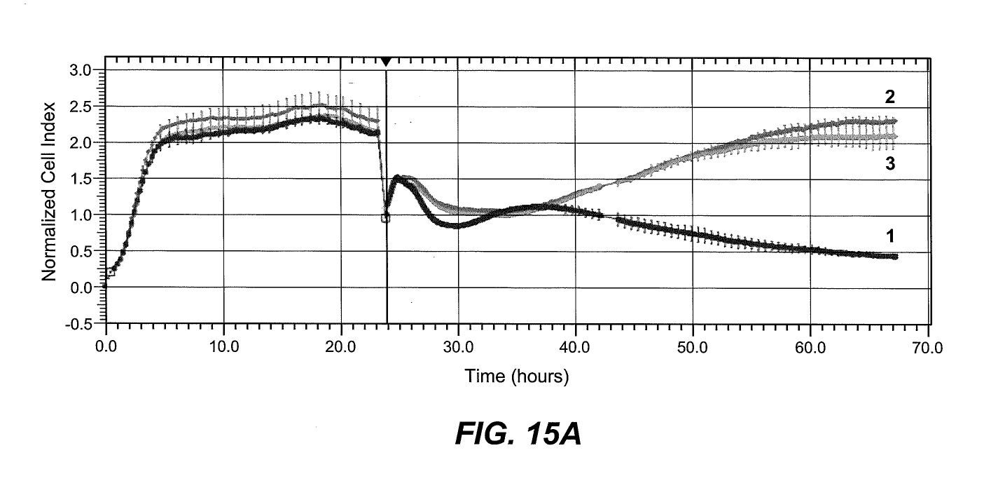

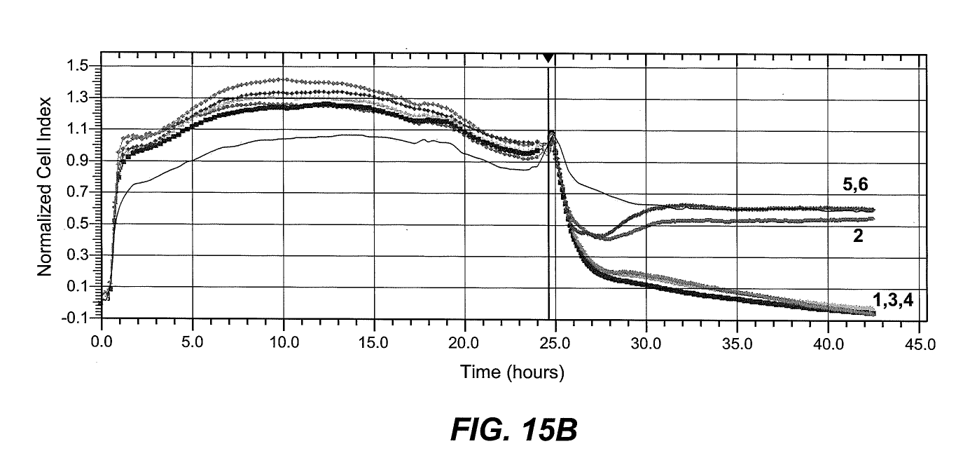

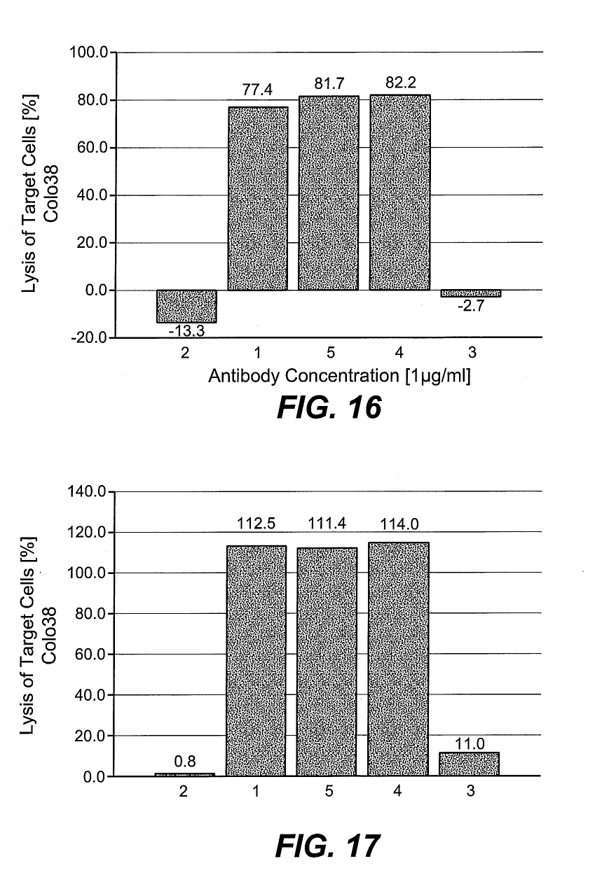

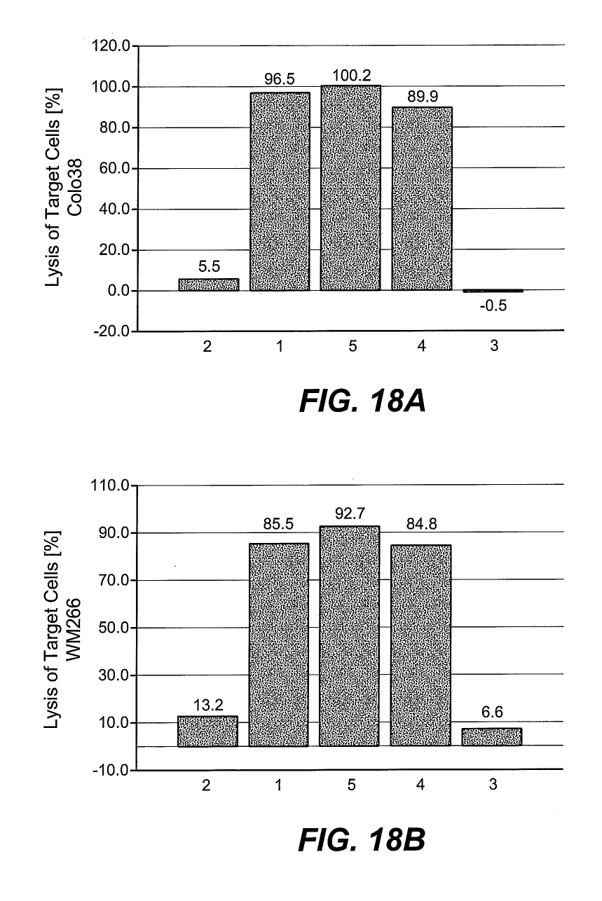

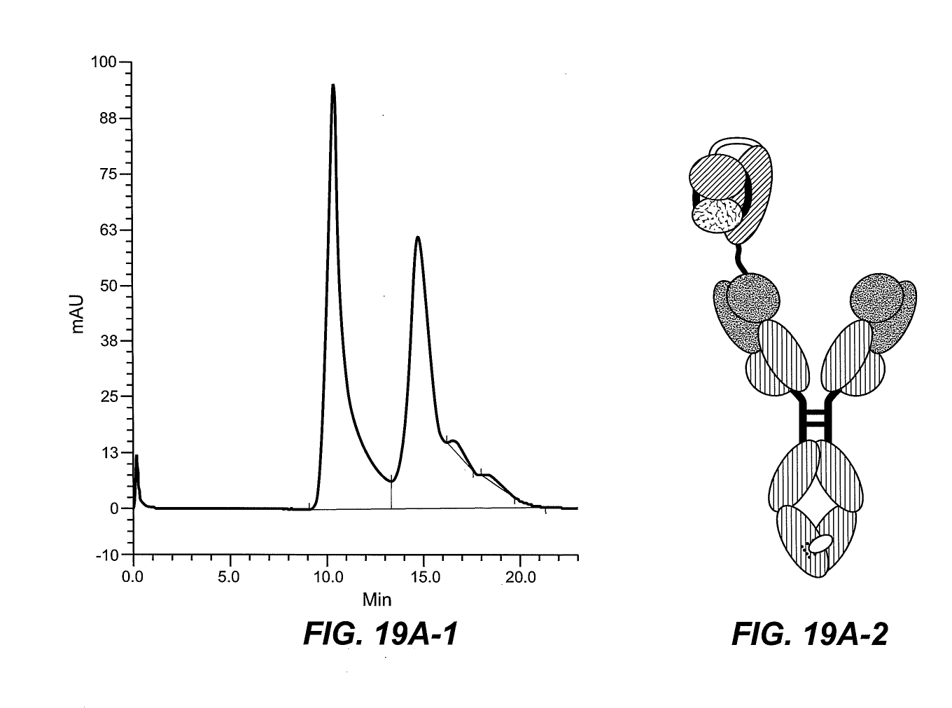

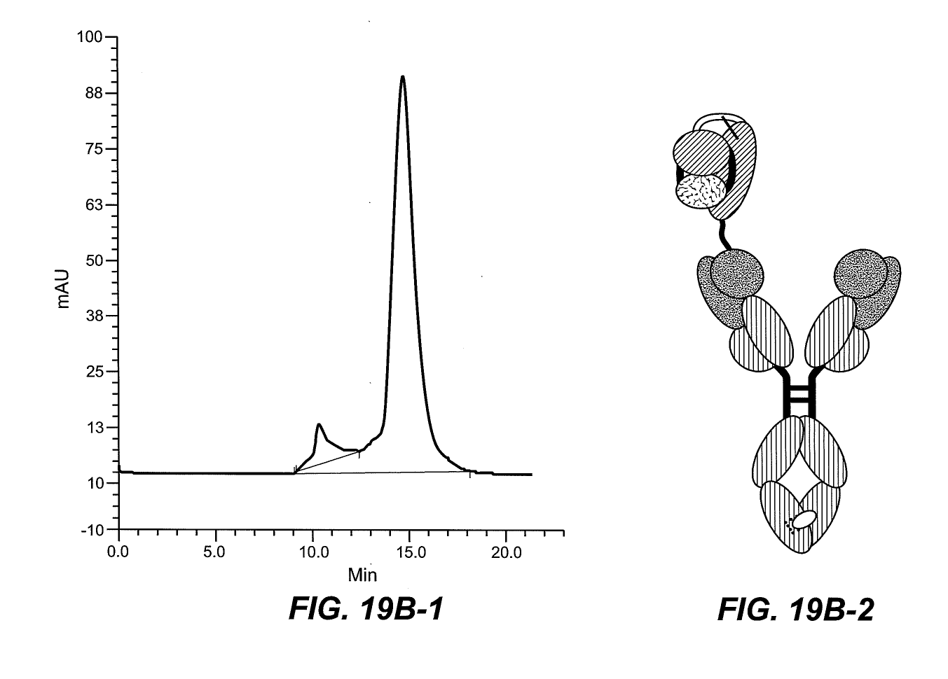

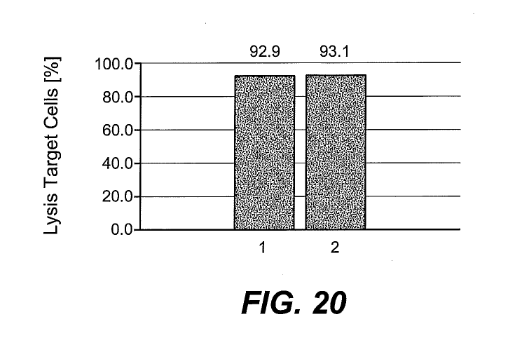

FIG. 6A-2): SDS-PAGE gel with Coomassie staining after protein A HPLC and SEC; reducing conditions; lane 1: molecular weight standard, lane 2: peptide-β2-microglobulin-HLA-A0201-HC+LC+IgG-Fc, lane 3: peptide-β2-microglobulin-HLA-A0201-HC+LC+ one-armed IgG (heavy and light chain).

FIG. 6B-1) Size exclusion chromatography chromatogram of peptide-β2-microglobulin-HLA-A0201-HC+LC+IgG-Fc; 1: high molecular weight forms (1.9 area %); 2: monomeric multi-function protein (98.1 area %).

FIG. 6B-2) Size exclusion chromatography chromatogram of peptide-β2-microglobulin-HLA-A0201-HC+LC+ one-armed IgG (heavy and light chain); 1: high molecular weight forms (2.1 area %); 2: monomeric multi-function protein (97.9 area %).

FIG. 6C: schematic molecules. FIG. 6C-1: peptide-β2-microglobulin-HLA-A0201-HC+LC+IgG-Fc, FIG. 6C-2: peptide-β2-microglobulin-HLA-A0201-HC+LC+ one-armed IgG.

FIG. 7 Binding of different MHC-I-multi function proteins on MCSP+ target cells (Colo38): Colo38 cells were incubated for 5 min. with Accutase (PAA, Cat.# L11-007) to obtain a single cell suspension. 2×10⁵ cells per vial were incubated with 1 μg/ml MHC-I-multi function protein in 100 μl PBS/2% FCS for 45 min. at 4° C. After incubation cells were washed with 1 ml cold PBS/2% FCS and centrifuged for 7 min. with 910 rpm. Cells were resuspended in 100 μl PBS/2% FCS with secondary antibody (goat anti-human IgG1 antibody PE conjugate, Jackson, Cat.#109-116-088) (2 μg/ml) and incubated for another 45 min. at 4° C. Cells were washed twice with 1 ml PBS %2% FCS and measured with BD Canto II Flow Cytometer.

FIGS. 8A-C Cytotoxicity assay: antigen binding multi-function protein as reported herein triggers lysis of H460M2 tumor cells through human CMV-specific T-cells. FIG. 8A) (6h) target Cells: CMV-specific effector T-cells 1:1.5; FIG. 8B) (6h) target cells:CMV-specific effector T-cells 1:0.75; FIG. 8C) (6h) target cells:CMV-specific effector T-cells 1:0.5; left bar: multi-function protein as reported herein; right bar MAB IGF-1R-afucosylated.

FIGS. 9A-C Cytotoxicity assay: antigen binding multi-function protein as reported herein triggers lysis of I24 3T3 tumor cells through human CMV-specific T-cells; FIG. 9A) (9h) Target Cells: CMV-specific Effector T-cells 1:1.5; FIG. 9B) (9h) Target Cells: CMV-specific Effector T-cells 1:0.75; FIG. 9C) 9h) Target Cells: CMV-specific Effector T-cells 1:0.5; left bar: multi-function protein as reported herein; middle bar: MAB IGF-1R-afu; right bar: MAB ed; right bar: anti-digoxygenin antibody.

FIGS. 10A-C FACS analysis of binding of anti-IGF-1R antibody and multi-function proteins as reported herein to lung adenocarcinoma cell line H460M2; FIG. 10A) secondary antibody only (goat anti-human IgG(H+L) (Jackson Laboratories, Cat#109-116-088));

FIG. 10B) multi-function protein as reported herein wherein the fusion polypeptide is fused to the N-terminus of the heavy chain of an anti-IGF-1R antibody comprising only one pair of variable domains; FIG. 10C) anti-IGF-1R antibody.

FIG. 11 In vitro efficacy and specificity (cytotoxicity assay) of different multi-function proteins as reported herein; a) multi-function protein comprising a monovalent anti-IGF1R antibody and a CMV-derived peptide; b) multi-function protein comprising a monovalent anti-IGF1R antibody and an EBV-derived peptide (control); c) multi-function protein comprising a bivalent anti-IGF1R antibody and a CMV-derived peptide; d) anti-IGF-1R antibody (control); e) anti-digoxigenin antibody (control).

FIG. 12 In vitro efficacy and specificity (EC50 value) of a multi-function protein as reported herein wherein the fusion polypeptide is fused to the N-terminus of the heavy chain of a complete anti-IGF-1R antibody determined at different target (T) to effector (E) cell ratios.

FIG. 13 Lysis of target cells after 6 hours incubation with a) a multi-function protein comprising a monovalent anti-IGF1R antibody and a fusion polypeptide comprising a CMV-derived peptide and b) an anti-IGF-1R antibody at a ratio of target to effector cells of 1:1.5.

FIG. 14 Course of normalized cell index for Colo38 cells incubated with MHC-I-anti-MCSP multi-function proteins; 1 μg/ml multi-function protein concentration (MHCI-0008 (1), MHCI-0010 (2), MHCI-0030 (3), MHCI-0031(4)), effector to target cell ratio of 10:1; PBMCs from Donor 3 (200.000 cells, Donor 3 is CMV-positive but EBV negative) and melanoma tumor cell line Colo38 (20.000 cells) and per 96 well, data are triplicates.

FIGS. 15A-B FIG. 15A: Course of normalized cell index for Colo38 cells incubated with MHC-I-anti-MCSP multi-function proteins; 1 μg/ml multi-function protein concentration (MHCI-0008 (1), MHCI-0010 (2), PBMCs only (3)), effector to target cell ratio of 10:1; PBMCs from Donor 3 (200.000 cells, Donor 3 is CMV-positive but EBV negative) and melanoma tumor cell line Colo38 (20.000 cells) and per 96 well, data are triplicates.

FIG. 15B: Course of normalized cell index for WM266 cells incubated with MHC-I-anti-MCSP multi-function proteins; 1 μg/ml multi-function protein concentration (MHCI-0008 (1), MHCI-0010 (2), MHCI-0030 (3), MHCI-0031 (4), target cells alone (5), target cells+T-cells (6)), effector to target cell ratio of 10:1; PBMCs from Donor 3 (200.000 cells, Donor 3 is CMV-positive but EBV negative) and melanoma tumor cell line MW266 (20.000 cells) and per 96 well, data are triplicates.

FIG. 16 Lysis of target cells after 42 hours of incubation with multi-function protein in the presence of non-stimulated PBMCs by the multi-function proteins MHCI-0008 (monovalent, CMV peptide loaded, 1), MHCI-0010 (monovalent, EBV peptide loaded control, 2), MHC-0026 (bivalent, CMV peptide loaded, non-binding control, 3), MHCI-0030 (monovalent, CMV peptide loaded, active, 4) and MHCI-0031 (bivalent, CMV peptide loaded, active, 5).

FIG. 17 LDH release after 48 hours of incubation with multi-function protein effected in the presence of non-stimulated PBMCs by the multi-function proteins MHCI-0008 (monovalent, CMV peptide loaded, 1), MHCI-0010 (monovalent, EBV peptide loaded control, 2), MHC-0026 (bivalent, CMV peptide loaded, non-binding control, 3), MHCI-0030 (monovalent, CMV peptide loaded, active, 4) and MHCI-0031 (bivalent, CMV peptide loaded, active, 5).

FIGS. 18A-B FIG. 18A: Lysis of Colo38 cells after 10 hours of incubation with multi-function protein in the presence of stimulated PBMCs by the multi-function proteins MHCI-0008 (monovalent, CMV peptide loaded, 1), MHCI-0010 (monovalent, EBV peptide loaded control, 2), MHC-0026 (bivalent, CMV peptide loaded, non-binding control, 3), MHCI-0030 (monovalent, CMV peptide loaded, active, 4) and MHCI-0031 (bivalent, CMV peptide loaded, active, 5) at a concentration of 1 μg/ml.

FIG. 18B: Lysis of WM266 cells after 10 hours of incubation with multi-function protein in the presence of stimulated PBMCs by the multi-function proteins MHCI-0008 (monovalent, CMV peptide loaded, 1), MHCI-0010 (monovalent, EBV peptide loaded control, 2), MHC-0026 (bivalent, CMV peptide loaded, non-binding control, 3), MHCI-0030 (monovalent, CMV peptide loaded, active, 4) and MHCI-0031 (bivalent, CMV peptide loaded, active, 5) at a concentration of 1 μg/ml.

FIGS. 19A-B FIG. 19A-1 and FIG. 19B-1: Analytical size exclusion chromatogram after protein A affinity chromatography but prior to preparative size exclusion chromatography of a non-disulfide stabilized multi-function protein (FIG. 19A-2) and a disulfide stabilized multi-function protein (FIG. 19B-2).

FIG. 20 Lysis of Colo38 cells with in the presence of stimulated PBMCs by the multi-function proteins MHCI-0031 (bivalent, CMV peptide loaded, active, non-disulfide-linked, 1) and MHCI-0054 (bivalent, CMV peptide loaded, active, disulfide-linked version of MHCI-0031, 2) at a concentration of 1 μg/ml.

### SHORT DESCRIPTION OF THE SEQUENCES

SEQ ID NO: 01 to 47 Human cytomegalovirus-derived peptide.
SEQ ID NO: 48 Human immunodeficiency virus-derived peptide.
SEQ ID NO: 49 Human herpesvirus 4 derived peptide.
SEQ ID NO: 50 Influenza A virus-derived peptide.
SEQ ID NO: 51 Hepatitis-B-virus-derived peptide.
SEQ ID NO: 52 Human T-cell lymphotropic virus type 1 derived peptide.
SEQ ID NO: 53 V-jun Sarcoma Virus 17 Oncogene Homolog (JUN) derived peptide.
SEQ ID NO: 54 Human adenovirus type 3-derived peptide.
SEQ ID NO: 55 Hepatitis-C-virus-derived peptide.
SEQ ID NO: 56 to 70 Dengue virus-derived peptides.
SEQ ID NO: 71 Human β2-microglobulin amino acid sequence.
SEQ ID NO: 72 Human HLA-A*0201 α1-α3 chain amino acid sequence.
SEQ ID NO: 73-84 Linker peptide amino acid sequences.
SEQ ID NO: 85 Human IgG1 CH2 domain amino acid sequence.
SEQ ID NO: 86 Human IgG1 CH2 domain amino acid sequence.
SEQ ID NO: 87 Human IgG1 Fc-region amino acid sequence.
SEQ ID NO: 88 Human IgG1 Fc-region L234A, L235A mutant amino acid sequence.
SEQ ID NO: 89 Human IgG1 Fc-region T366S, L368A, Y407V mutant amino acid sequence.
SEQ ID NO: 90 Human IgG1 Fc-region T366W mutant amino acid sequence.
SEQ ID NO: 91 Human IgG1 Fc-region L234A, L235A, T366S, L368A, Y407V mutant amino acid sequence.
SEQ ID NO: 92 Human IgG1 Fc-region L234A, L235A, T366W mutant amino acid sequence.
SEQ ID NO: 93 Human IgG1 Fc-region P329G mutant amino acid sequence.
SEQ ID NO: 94 Human IgG1 Fc-region L234A, L235A, P329G mutant amino acid sequence.
SEQ ID NO: 95 Human IgG1 Fc-region P329G, T366S, L368A, Y407V mutant amino acid sequence.
SEQ ID NO: 96 Human IgG1 Fc-region P329G, T366W mutant amino acid sequence.
SEQ ID NO: 97 Human IgG1 Fc-region L234A, L235A, P329G, T366S, L368A, Y407V mutant amino acid sequence.
SEQ ID NO: 98 Human IgG1 Fc-region L234A, L235A, P329G, T366W mutant amino acid sequence.
SEQ ID NO: 99 Human IgG4 Fc-region amino acid sequence.
SEQ ID NO: 100 Human IgG4 Fc-region S228P, L235E mutant amino acid sequence.
SEQ ID NO: 101 Human IgG4 Fc-region S228P, L235E, P329G mutant amino acid sequence.
SEQ ID NO: 102 Human IgG4 Fc-region S228P, L235E, P329G, T366S, L368A, Y407V mutant amino acid sequence.
SEQ ID NO: 103 Human IgG4 Fc-region S228P, L235E, P329G, T366W mutant amino acid sequence.
SEQ ID NO: 104 HVR-L1
SEQ ID NO: 105 HVR-L2
SEQ ID NO: 106 HVR-L3
SEQ ID NO: 107 HVR-L1
SEQ ID NO: 108 HVR-H1
SEQ ID NO: 109 HVR-H2
SEQ ID NO: 110 HVR-H3
SEQ ID NO: 111 HVR-H1
SEQ ID NO: 112 HVR-H2
SEQ ID NO: 113 VL
SEQ ID NO: 114 VH
SEQ ID NO: 115 VL
SEQ ID NO: 116 VH
SEQ ID NO: 117 MHC-I-VH (MCSP)-IgG1 Fc-region L234A, L235A, P329G, T366S, L368A, Y407V mutant amino acid sequence.
SEQ ID NO: 118 VH(MCSP)-IgG1 Fc-region L234A, L235A, P329G, T366W mutant amino acid sequence.
SEQ ID NO: 119 VL(MCSP)-CL amino acid sequence.
SEQ ID NO: 120 Humanized anti-IGF-1R monoclonal light chain antibody amino acid sequence (kappa).
SEQ ID NO: 121 Humanized anti-IGF-1R monoclonal heavy chain antibody amino acid sequence (IgG1 L234A, L235A mutant).
SEQ ID NO: 122 Humanized anti-IGF-1R monoclonal heavy chain antibody amino acid sequence (IgG1 L234A, L235A mutant and knob variant).
SEQ ID NO: 123 Humanized anti-IGF-1R monoclonal heavy chain antibody amino acid sequence (IgG1 L234A, L235A mutant and hole variant).
SEQ ID NO: 124 Human IgG1 Fc-region mutant hinge region and L234A, L235A mutant and knob variant.
SEQ ID NO: 125 Disulfide-stabilized single chain Fv of humanized anti-IGF-1R monoclonal antibody.
SEQ ID NO: 126 Murine anti-MCSP monoclonal light chain antibody amino acid sequence (kappa).
SEQ ID NO: 127 Humanized anti-MCSP monoclonal light chain antibody amino acid sequence (kappa).
SEQ ID NO: 128 Murine anti-MCSP monoclonal heavy chain antibody amino acid sequence (IgG1 L234A, L235A mutant).
SEQ ID NO: 129 Humanized anti-MCSP monoclonal heavy chain antibody amino acid sequence (IgG1 L234A, L235A mutant).
SEQ ID NO: 130 Murine anti-MCSP monoclonal heavy chain antibody amino acid sequence (IgG1 L234A, L235A mutant and knob variant).
SEQ ID NO: 131 Humanized anti-MCSP monoclonal heavy chain antibody amino acid sequence (IgG1 L234A, L235A mutant and knob variant).
SEQ ID NO: 132 Murine anti-MCSP monoclonal heavy chain antibody amino acid sequence (IgG1 L234A, L235A mutant and hole variant).
SEQ ID NO: 133 Humanized anti-MCSP monoclonal heavy chain antibody amino acid sequence (IgG1 L234A, L235A mutant and hole variant).
SEQ ID NO: 134 Disulfide-stabilized single chain Fv of murine anti-MCSP monoclonal antibody.
SEQ ID NO: 135 Disulfide-stabilized single chain Fv of humanized anti-MCSP monoclonal antibody.
SEQ ID NO: 136 Linker peptide 13.
SEQ ID NO: 137 Disulfide-stabilized MHC-I-VH (MCSP)-IgG1 Fc-region L234A, L235A, P329G, T366S, L368A, Y407V mutant amino acid sequence.
SEQ ID NO: 138 Amino acid sequence of human MCSP.

### I. DEFINITIONS

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

The term “amino acid” as used within this application denotes the group of carboxy α-amino acids, which directly or in form of a precursor can be encoded by a nucleic acid. The individual amino acids are encoded by nucleic acids consisting of three nucleotides, so called codons or base-triplets. Each amino acid is encoded by at least one codon. This is known as “degeneration of the genetic code”. The term “amino acid” as used within this application denotes the naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

The terms “anti-target antibody” and “an antibody that binds to a target” refer to an antibody that is capable of binding a target with sufficient affinity such that the antibody is useful as a diagnostic and/or therapeutic agent in targeting the target. In certain embodiments, an antibody that binds to the target has a dissociation constant (Kd) of ≦10 nM, ≦1 nM, ≦0.1 nM, ≦0.01 nM, or ≦0.001 nM (e.g. 10⁻⁸ M or less, e.g. from 10⁻⁸ M to 10⁻¹³ M, e.g., from 10⁻⁹ M to 10⁻¹³ M).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); single domain antibodies; and multispecific antibodies formed from antibody fragments.

The term “antigen binding site” denotes a proteinaceous moiety that can specifically bind to a target. Exemplary antigen binding sites are peptides, antibody fragments, domain antibodies, or variable domains of single chain antibodies (e.g. camel or shark antibodies). The antigen binding site can be a naturally occurring antigen binding site or an engineered antigen binding site. Exemplary engineered antigen binding sites are DARPINs, domain exchanged antibodies or domain exchanged antibody fragments, and dual variable domain antibodies.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The term “class I MHC molecule with a relative frequency of” denotes that the respective class I MHC molecule has a frequency of occurrence in a specific population of humans or within all humans of the given relative frequency. That is a class I MHC molecule with a relative frequency of 10% or more can be found in 10% or more of all humans of a specific population, such as e.g. in 27.2% of all humans of European origin.

The term “comprising” as used herein encompasses the tem “consisting”, i.e. a change from comprising to consisting is considered to be a limitation.

The “conjugation” of a multi-function protein to its conjugation partner can be performed by different methods, such as chemical binding, or binding via a specific binding pair. The term “conjugation partner” denotes e.g. polypeptides, detectable labels, members of specific binding pairs. In one embodiment the conjugation of multi-function protein to its conjugation partner is performed by chemically binding via N-terminal and/or ε-amino groups (lysine), ε-amino groups of different lysins, carboxy-, sulfhydryl-, hydroxyl-, and/or phenolic functional groups of the amino acid sequence of the parts of the multi-function protein, and/or sugar alcohol groups of the carbohydrate structure of the multi-function protein. In one embodiment the multi-function protein is conjugated to its conjugation partner via a specific binding pair.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents.

Chromogens (fluorescent or luminescent groups and dyes), enzymes, NMR-active groups or metal particles, haptens, e.g. digoxigenin, are examples of “detectable labels”. The detectable label can also be a photoactivatable crosslinking group, e.g. an azido or an azirine group. Metal chelates which can be detected by electrochemiluminescense are also suitable signal-emitting groups, with particular interest being given to ruthenium chelates, e.g. a ruthenium (bispyridyl)₃²⁺ chelate. Suitable ruthenium labeling groups are described, for example, in EP 0 580 979, WO 90/05301, WO 90/11511, and WO 92/14138. For direct detection the labeling group can be selected from any known detectable marker groups, such as dyes, luminescent labeling groups such as chemiluminescent groups, e.g. acridinium esters or dioxetanes, or fluorescent dyes, e.g. fluorescein, coumarin, rhodamine, oxazine, resorufin, cyanine and derivatives thereof. Other examples of labeling groups are luminescent metal complexes, such as ruthenium or europium complexes, enzymes, e.g. as used for ELISA or for CEDIA (Cloned Enzyme Donor Immunoassay, e.g. EP-A-0 061 888), and radioisotopes.

“Effector functions” refer to those biological activities attributable to the Fc-region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “expression” as used herein refers to transcription and/or translation and secretion processes occurring within a cell. The level of transcription of a nucleic acid sequence of interest in a cell can be determined on the basis of the amount of corresponding mRNA that is present in the cell. For example, mRNA transcribed from a sequence of interest can be quantitated by RT-PCR or by Northern hybridization (see Sambrook, J., et al., Molecular Cloning: A Laboratory Manual, Second Edition, Cold Spring Harbor Laboratory Press, Cold Spring Harbor, N.Y. (1989)). Polypeptides encoded by a nucleic acid can be quantitated by various methods, e.g. by ELISA, by assaying for the biological activity of the polypeptide, or by employing assays that are independent of such activity, such as Western blotting or radioimmunoassay, using immunoglobulins that recognize and bind to the polypeptide (see Sambrook, et al., (1989), supra).

An “expression cassette” denotes a construct that contains the necessary regulatory elements, such as promoter and polyadenylation site, for expression of at least the contained nucleic acid in a cell.

The term “expression machinery” denotes the sum of the enzymes, cofactors, etc. of a cell that is involved in the steps beginning with the transcription step of a nucleic acid or gene (i.e. also called “gene expression machinery”) to the post-translational modification of the polypeptide encoded by the nucleic acid. The expression machinery e.g. comprises the steps of transcription of DNA into pre-mRNA, pre-mRNA splicing to mature mRNA, translation into a polypeptide of the mRNA, and post translational modification of the polypeptide.

An “expression plasmid” is a nucleic acid providing all required elements for the expression of the comprised structural gene(s) in a host cell. Typically, an expression plasmid comprises a prokaryotic plasmid propagation unit, e.g. for E. coli, comprising an origin of replication, and a selectable marker, an eukaryotic selection marker, and one or more expression cassettes for the expression of the structural gene(s) of interest each comprising a promoter, a structural gene, and a transcription terminator including a polyadenylation signal. Gene expression is usually placed under the control of a promoter, and such a structural gene is said to be “operably linked to” the promoter. Similarly, a regulatory element and a core promoter are operably linked if the regulatory element modulates the activity of the core promoter.

The term “Fc-region” denotes the C-terminal region of an immunoglobulin heavy chain. The Fc-region is a dimeric molecule comprising two disulfide-linked antibody heavy chain polypeptides. An Fc-region can be generated by papain digestion, or IdeS digestion, or trypsin digestion of an intact (full length) antibody or can be produced recombinantly.

The Fc-region obtainable from a full length antibody or immunoglobulin comprises at least residues 226 (Cys) to the C-terminus of the full length heavy chain and, thus, comprises a part of the hinge region and two or three constant domains, i.e. a CH2 domain, a CH3 domain, and an additional/extra CH4 domain in case of IgE and IgM class antibodies. However, the C-terminal lysine (Lys447) of the Fc-region may or may not be present. Unless otherwise specified herein, numbering of amino acid residues in the Fc-region or constant region is according to the EU numbering system, also called the EU index, as described in Kabat, E. A., et al., Sequences of Proteins of Immunological Interest, 5th ed., Public Health Service, National Institutes of Health, Bethesda, Md. (1991), NIH Publication 91-3242.

The formation of the dimeric Fc-region comprising two identical or non-identical antibody heavy chain Fc-region polypeptides is mediated by the non-covalent dimerization of the comprised CH3 domains (for involved amino acid residues see e.g. Dall'Acqua, W., Biochem. 37 (1998) 9266-9273). The Fc-region is covalently stabilized by the formation of disulfide bonds in the hinge region (see e.g. Huber, R., et al., Nature 264 (1976) 415-420; Thies, M. J., et al., J. Mol. Biol. 293 (1999) 67-79).

It is known from U.S. Pat. No. 5,648,260 and U.S. Pat. No. 5,624,821 that the modification of defined amino acid residues in the Fc-region results in phenotypic effects.

The multi-function protein as reported herein may comprise in one embodiment as antibody heavy chain hinge region polypeptide a human Fc-region or an Fc-region derived from human origin. In a further embodiment the Fc-region is either an Fc-region of a human antibody of the subclass IgG4 or an Fc-region of a human antibody of the subclass IgG1, IgG2, or IgG3, which is modified in such a way that no Fcγ receptor (e.g. FcγRIIIa) binding and/or no C1q binding can be detected. In one embodiment the Fc-region is a human Fc-region and especially either from human IgG4 subclass or a mutated Fc-region from human IgG1 subclass. In one embodiment the Fc-region is from human IgG1 subclass with mutations L234A and L235A and P329G. While IgG4 shows reduced Fcγ receptor (FcγRIIIa) binding, antibodies of other IgG subclasses show strong binding. However Pro238, Asp265, Asp270, Asn297 (loss of Fc carbohydrate), Pro329, Leu234, Leu235, Gly236, Gly237, Ile253, Ser254, Lys288, Thr307, Gln311, Asn434, or/and His435 are residues which, if altered, provide also reduced Fcγ receptor binding (Shields, R. L., et al., J. Biol. Chem. 276 (2001) 6591-6604; Lund, J., et al., FASEB J. 9 (1995) 115-119; Morgan, A., et al., Immunology 86 (1995) 319-324; EP 0 307 434). In one embodiment a multi-function protein as reported herein is in regard to Fcγ receptor binding of IgG4 subclass or of IgG1 or IgG2 subclass, with a mutation in L234, L235, P329 and/or D265, and/or contains the PVA236 mutation. In one embodiment the mutations are S228P, L234A, L235A, L235E, PVA236 (PVA236 denotes that the amino acid sequence ELLG (given in one letter amino acid code) from amino acid position 233 to 236 of IgG1 or EFLG of IgG4 is replaced by PVA) and/or P329G. In one embodiment the mutations are S228P and P329G of IgG4, and L234A, L235A and P329G of IgG1. The Fc-region of an antibody is directly involved in ADCC (antibody-dependent cell-mediated cytotoxicity) and CDC (complement-dependent cytotoxicity). A multi-function protein which does not bind Fcγ receptor and/or complement factor C1q does not elicit antibody-dependent cellular cytotoxicity (ADCC) and/or complement dependent cytotoxicity (CDC).

A polypeptide chain of a wild-type human Fc-region of the IgG1 isotype has the following amino acid sequence:

(SEQ ID NO: 87)
EPKSADKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK.

A polypeptide chain of a variant human Fc-region of the IgG1 isotype with the mutations L234A, L235A has the following amino acid sequence:

(SEQ ID NO: 88)
EPKSADKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK.

A polypeptide chain of a variant human Fc-region of the IgG1 isotype with T366S, L368A and Y407V mutations has the following amino acid sequence:

(SEQ ID NO: 89)
EPKSADKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVCTLPPSRDELTKNQVSL
SCAVKGFYPSDIAVEWESNGQPENNYKTTPPVLDSDGSFFLVSKLTVDKS
RWQQGNVFSCSVMHEALHNHYTQKSLSLSPGK.

A polypeptide chain of a variant human Fc-region of the IgG1 isotype with a T366W mutation has the following amino acid sequence:

(SEQ ID NO: 90)
EPKSADKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPCRDELTKNQVSL
WCLVKGFYPSDIAVEWESNGQPENNYKTTPPVLDSDGSFFLYSKLTVDKS
RWQQGNVFSCSVMHEALHNHYTQKSLSLSPGK.

A polypeptide chain of a variant human Fc-region of the IgG1 isotype with a L234A, L235A and T366S, L368A and Y407V mutations has the following amino acid sequence:

(SEQ ID NO: 91)
EPKSADKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVCTLPPSRDELTKNQVSL
SCAVKGFYPSDIAVEWESNGQPENNYKTTPPVLDSDGSFFLVSKLTVDKS
RWQQGNVFSCSVMHEALHNHYTQKSLSLSPGK.

A polypeptide chain of a variant human Fc-region of the IgG1 isotype with a L234A, L235A and T366W mutation has the following amino acid sequence:

(SEQ ID NO: 92)
EPKSADKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPCRDELTKNQVSL
WCLVKGFYPSDIAVEWESNGQPENNYKTTPPVLDSDGSFFLYSKLTVDKS
RWQQGNVFSCSVMHEALHNHYTQKSLSLSPGK.

A polypeptide chain of a variant human Fc-region of the IgG1 isotype with a P329G mutation has the following amino acid sequence:

(SEQ ID NO: 93)
EPKSADKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALGAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK.

A polypeptide chain of a variant human Fc-region of the IgG1 isotype with a L234A, L235A and P329G mutation has the following amino acid sequence:

(SEQ ID NO: 94)
EPKSADKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALGAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK.

A polypeptide chain of a variant human Fc-region of the IgG1 isotype with a P239G and T366S, L368A and Y407V mutation has the following amino acid sequence:

(SEQ ID NO: 95)
EPKSADKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALGAPIEKTISKAKGQPREPQVCTLPPSRDELTKNQVSL
SCAVKGFYPSDIAVEWESNGQPENNYKTTPPVLDSDGSFFLVSKLTVDKS
RWQQGNVFSCSVMHEALHNHYTQKSLSLSPGK.

A polypeptide chain of a variant human Fc-region of the IgG1 isotype with a P329G and T366W mutation has the following amino acid sequence:

(SEQ ID NO: 96)
EPKSADKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALGAPIEKTISKAKGQPREPQVYTLPPCRDELTKNQVSL
WCLVKGFYPSDIAVEWESNGQPENNYKTTPPVLDSDGSFFLYSKLTVDKS
RWQQGNVFSCSVMHEALHNHYTQKSLSLSPGK.

A polypeptide chain of a variant human Fc-region of the IgG1 isotype with a L234A, L235A, P329G and T366S, L368A and Y407V mutation has the following amino acid sequence:

(SEQ ID NO: 97)
EPKSADKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALGAPIEKTISKAKGQPREPQVCTLPPSRDELTKNQVSL
SCAVKGFYPSDIAVEWESNGQPENNYKTTPPVLDSDGSFFLVSKLTVDKS
RWQQGNVFSCSVMHEALHNHYTQKSLSLSPGK.

A polypeptide chain of a variant human Fc-region of the IgG1 isotype with a L234A, L235A, P329G and T366W mutation has the following amino acid sequence:

(SEQ ID NO: 98)
EPKSADKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALGAPIEKTISKAKGQPREPQVYTLPPCRDELTKNQVSL
WCLVKGFYPSDIAVEWESNGQPENNYKTTPPVLDSDGSFFLYSKLTVDKS
RWQQGNVFSCSVMHEALHNHYTQKSLSLSPGK.

A polypeptide chain of a wild-type human Fc-region of the IgG4 isotype has the following amino acid sequence:

(SEQ ID NO: 99)
ESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK.

A polypeptide chain of a variant human Fc-region of the IgG4 isotype with a S228P and L235E mutation has the following amino acid sequence:

(SEQ ID NO: 100)
ESKYGPPCPPCPAPEFEGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK.

A polypeptide chain of a variant human Fc-region of the IgG4 isotype with a S228P, L235E and P329G mutation has the following amino acid sequence:

(SEQ ID NO: 101)
ESKYGPPCPPCPAPEFEGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLGSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK.

A polypeptide chain of a variant human Fc-region of the IgG4 isotype with a S228P, L235E, P329G and T366S, L368A and Y407V mutation has the following amino acid sequence:

(SEQ ID NO: 102)
ESKYGPPCPPCPAPEFEGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLGSSIEKTISKAKGQPREPQVYTLPPSQEEMTKNQVSLSCA
VKGFYPSDIAVEWESNGQPENNYKTTPPVLDSDGSFFLVSRLTVDKSRWQ
EGNVFSCSVMHEALHNHYTQKSLSLSLGK.

A polypeptide chain of a variant human Fc-region of the IgG4 isotype with a S228P, L235E, P329G and T366W mutation has the following amino acid sequence:

(SEQ ID NO: 103)
ESKYGPPCPPCPAPEFEGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLGSSIEKTISKAKGQPREPQVYTLPPSQEEMTKNQVSLWCL
VKGFYPSDIAVEWESNGQPENNYKTTPPVLDSDGSFFLYSRLTVDKSRWQ
EGNVFSCSVMHEALHNHYTQKSLSLSLGK.

The terms “host cell”, “host cell line”, and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “cell” includes both prokaryotic cells, which are used for propagation of plasmids, and eukaryotic cells, which are used for the expression of a nucleic acid. In one embodiment the eukaryotic cell is a mammalian cell. In one embodiment the mammalian cell is selected from the group of mammalian cells comprising CHO cells (e.g. CHO K1, CHO DG44), BHK cells, NS0 cells, Sp2/0 cells, HEK 293 cells, HEK 293 EBNA cells, PER.C6® cells, and COS cells.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

An “immunoconjugate” denotes a multi-function protein as reported herein conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” multi-function protein is one which has been separated from a component of its natural environment. In some embodiments, a multi-function protein is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “MCSP”, as used herein, refers to any native MCSP (Melanoma Chondroitin Sulfate Proteoglycan) from any vertebrate source, including mammals such as primates (e.g. humans) and rodents (e.g., mice and rats), unless otherwise indicated. The term encompasses “full-length”, unprocessed MCSP as well as any form of MCSP that results from processing in the cell. The term also encompasses naturally occurring variants of MCSP, e.g., splice variants or allelic variants. MCSP is also known as chondroitin sulfate proteoglycan 4 (CSPG4), chondroitin sulfate proteoglycan NG2, high molecular weight-melanoma associated antigen (HMW-MAA), and melanoma chondroitin sulfate proteoglycan. The amino acid sequence of an exemplary human MCSP is shown in SEQ ID NO: 1. See also Pluschke, G., et al., Proc. Natl. Acad. Sci. USA 93 (1996) 9710-9715, Staub, E., et al., FEBS Lett. 527 (2002) 114-118, and GenBank Accession No: NP_001888.

The term “one antigen presenting domain” denotes exactly one, i.e. a single, antigen presenting domain as defined and excludes the presence of a further, i.e. second, antigen presenting domains defined. The term “one” denotes “exactly one” or “a single”.

“Operably linked” refers to a juxtaposition of two or more components, wherein the components so described are in a relationship permitting them to function in their intended manner. For example, a promoter and/or enhancer are operably linked to a coding sequence, if it acts in cis to control or modulate the transcription of the linked sequence. Generally, but not necessarily, the DNA sequences that are “operably linked” are contiguous and, where necessary to join two protein encoding regions such as a secretory leader and a polypeptide, contiguous and in (reading) frame. However, although an operably linked promoter is generally located upstream of the coding sequence, it is not necessarily contiguous with it. Enhancers do not have to be contiguous. An enhancer is operably linked to a coding sequence if the enhancer increases transcription of the coding sequence. Operably linked enhancers can be located upstream, within or downstream of coding sequences and at considerable distance from the promoter. A polyadenylation site is operably linked to a coding sequence if it is located at the downstream end of the coding sequence such that transcription proceeds through the coding sequence into the polyadenylation sequence. A translation stop codon is operably linked to an exonic nucleic acid sequence if it is located at the downstream end (3′ end) of the coding sequence such that translation proceeds through the coding sequence to the stop codon and is terminated there. Linking is accomplished by recombinant methods known in the art, e.g., using PCR methodology and/or by ligation at convenient restriction sites. If convenient restriction sites do not exist, then synthetic oligonucleotide adaptors or linkers are used in accord with conventional practice.

The term “package insert” is used to refer to instructions customarily included in commercial packages of therapeutic products, that contain information about the indications, usage, dosage, administration, combination therapy, contraindications and/or warnings concerning the use of such therapeutic products.

The term “peptide linker” denotes amino acid sequences of natural and/or synthetic origin. They consist of a linear amino acid chain wherein the 20 naturally occurring amino acids are the monomeric building blocks. The peptide linker has a length of from 1 to 50 amino acids, in one embodiment between 1 and 28 amino acids, in a further embodiment between 2 and 25 amino acids. The peptide linker may contain repetitive amino acid sequences or sequences of naturally occurring polypeptides. The linker has the function to ensure that polypeptides conjugated to each other can perform their biological activity by allowing the polypeptides to fold correctly and to be presented properly. In one embodiment the peptide linker is rich in glycine, glutamine, and/or serine residues. These residues are arranged e.g. in small repetitive units of up to five amino acids, such as GS (SEQ ID NO: 73), GGS (SEQ ID NO: 74), GGGS (SEQ ID NO: 75), and GGGGS (SEQ ID NO: 80). This small repetitive unit may be repeated for one to five times. At the amino- and/or carboxy-terminal ends of the multimeric unit up to six additional arbitrary, naturally occurring amino acids may be added. Other synthetic peptidic linkers are composed of a single amino acid, which is repeated between 10 to 20 times and may comprise at the amino- and/or carboxy-terminal end up to six additional arbitrary, naturally occurring amino acids. All peptidic linkers can be encoded by a nucleic acid molecule and therefore can be recombinantly expressed. As the linkers are themselves peptides, the polypeptide connected by the linker are connected to the linker via a peptide bond that is formed between two amino acids.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

A “polypeptide” is a polymer consisting of amino acids joined by peptide bonds, whether produced naturally or synthetically. Polypeptides of less than about 25 amino acid residues may be referred to as “peptides”, whereas molecules consisting of two or more polypeptides or comprising one polypeptide of more than 100 amino acid residues may be referred to as “proteins”. A polypeptide may also comprise non-amino acid components, such as carbohydrate groups, metal ions, or carboxylic acid esters. The non-amino acid components may be added by the cell, in which the polypeptide is expressed, and may vary with the type of cell. Polypeptides are defined herein in terms of their amino acid backbone structure or the nucleic acid encoding the same. Additions such as carbohydrate groups are generally not specified, but may be present nonetheless.

A “structural gene” denotes the region of a gene without a signal sequence, i.e. the coding region.

The term “T-cell response eliciting peptide” denotes a peptide that is presented in the peptide-binding grove of a class I MHC multi-function protein and which is recognized by circulating memory or effector T-cells. Recognition of the peptide results in an immune response effecting the removal of the cell presenting such a peptide-class I MHC multi-function protein.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt, T. J., et al., Kuby Immunology, 6th ed., W.H. Freeman and Co., N.Y. (2007), page 91) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano, S., et al., J. Immunol. 150 (1993) 880-887; Clackson, T., et al., Nature 352 (1991) 624-628).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### II. COMPOSITIONS AND METHODS

During recombinant production covalent peptide-MHC-immunoglobulin conjugates cannot be expressed at levels comparable to normal full length antibodies. This is only possible for certain specific formats. Full length antibody (IgG)-MHC fusions cannot be expressed in bacteria. Further, full length antibody (IgG) peptide-MHC-fusions cannot be expressed at significant levels. So far only fusions of antibody fragments (scFv and Fab, lacking a hinge and an Fc-region) could be expressed as MHC class I fusions in bacteria (preferably in E. coli). The expression was only successful via inclusion bodies followed by a complex refolding procedure which is a technical difficult process especially at larger scale.

An alternative is the recombinant expression of soluble MHC complexes in bacteria as such, i.e. without being fused to a full length antibody or antibody fragment, followed by a chemical conjugation or Biotin-Streptavidin mediated non-covalent coupling to an antibody fragment. Chemical conjugations are not site specific and lead to a large product heterogeneity compared to recombinantly produced fusion polypeptides.

Expression in eukaryotic was limited so far due to no or low expression levels. So far the only expression in mammalian cells is described in Greten, T. F. et al. (J. Immunol. Meth. 271 (2002) 125-135). However, the obtained yields are very low, and actually too low for a technical process and much lower than for normal antibodies.

It has now been found that full length antibodies (IgG) fused to peptide-MHC complexes can be recombinantly expressed at high levels comparable to normal antibodies when the fusion polypeptide carries only a single MHC complex.

A. Exemplary Multi-Function Protein

Herein is reported an antigen binding multi-function protein comprising as first part an antibody derived part that specifically binds to a target antigen, and as second part a virus-derived peptide linked to a MHC class I protein complex. If the multi-function protein as reported herein comprises one or more further antigen presenting domain(s) these further antigen presenting domains do not comprise an MHC molecule. I.e. the multi-function protein as reported herein comprises exactly one antigen presenting domain comprising an MHC molecule.

The term “MHC molecule” denotes a fusion polypeptide comprising the extracellular domains α1, α2, and α3 of a class I MHC molecule. In one embodiment the extracellular domains α1, α2, and α3 are of a human class I MHC molecule.

With the multi-function protein as reported herein existing virus-specific circulating cytotoxic T-cells (T-memory-cells and/or T-effector-cells) of an individual can be directed to cells expressing the target antigen, to which the antibody derived part of the multi-function protein specifically binds to, by dressing these cells with MHC class I multi-function complexes mimicking an acute viral infection.

In one aspect, the invention is based, in part, on the finding that a multi-function protein as reported herein, which comprises as first part a virus-derived peptide linked to a MHC class I protein and as second part an antibody derived disulfide-linked molecule, can be used to direct existing virus-specific cytotoxic T-cells of an individual to cells expressing a target antigen mimicking an acute viral infection and thereby removal of the cells expressing the target antigen can be initiated.

In certain embodiments a multi-function protein comprising an antigen presenting domain comprising (i) a virus-derived peptide, (ii) the soluble HLA-A allele A*0201, and (iii) beta-2-microglobulin, is provided.

Multi-function proteins as reported herein are useful, e.g., for the diagnosis or treatment of various diseases like cancer or viral infections.

In one aspect, the invention provides a multi-functions protein that binds (i) to a cell surface antigen and (ii) to cytotoxic T-cells.

The multi-function proteins as reported herein exploit a naturally occurring, highly effective anti-viral immune response to remove/disintegrate target cells, e.g. tumor cells or virus infected cells. The cell removal is achieved by using an individual's own very powerful circulating T-cells that do not need any co-stimulation for their activation. Additionally a small number of therapeutic molecules are needed on the cell surface for mechanism of action (see e.g. Mottez, E., et al., J. Exp. Med. 181 (1995) 493-502).

During the treatment the multi-function protein can trigger the anti-viral immune response of the individual similar to an immunization. Thereby multiple treatments/applications can enhance the efficacy of the treatment. Thus, an immunization as pretreatment can be used in order to enhance efficacy.

Also an allotype can be used whose frequency within the population is very low, as in one embodiment below 1%. The use of such an allotype may make an immunization step obsolete as the allotype will be recognized by the individual's immune system as foreign and an immune response will be initiated.

The targeting antigen binding site needs to be highly cell or antigen specific to limit toxicity and side effects.

Thus by using a multi-function protein as reported herein
(i) only a highly specific T-cell population is activated (CD8 positive effector/memory cells specific for a single virus-derived peptide displayed in the MHC-I protein complex of the multi-function protein), all other CD3⁺-cells are not affected (CD4⁺-T-cells: TH1, TH2, TH17, regulatory T-cells);
(ii) the natural response of the individual's immune system is mimicked (normal removal of virus-infected cells); and
(iii) the response to the application/treatment of/with the multi-function protein will be low at the beginning but can boost during treatment (specific T-cells will be activated and expand in number), therewith initially infusion reactions and initial cytokine release can be reduced.

In one embodiment the method comprises the step of stimulating CD8-positive cytotoxic T-cell by application of a selected virus-derived peptide, e.g. a human cytomegalovirus (huCMV) derived peptide. In one embodiment the peptide has the amino acid sequence of SEQ ID NO: 01.

It has been shown that the activated CMV-peptide specific T-cells mediate effective tumor cell removal in vitro (tumor cells loaded with the CMV-derived peptide in vitro).

Virus-infected cells present a complex of virus-derived peptides with MHC class I proteins on their cell surface. These are recognized by specific CD8⁺ T-cells which remove/deplete the virus-derived peptide presenting cells. Cytolytic (cytotoxic) CD8⁺-T-cells (CTL) recognize peptides in MHC class I proteins by their specific T-cell-receptor. The CTLs trigger removal of virus infected cells without the requirement of a co-stimulating signal.

Effector cells, e.g. peripheral blood mononuclear cells (PBMC) or FACS-sorted CD8⁺-T-cells, which can be pre-stimulated with the CMV-derived peptide as comprised in the fusion polypeptide as reported herein can be used.

The HLA-allotype of an individual to be treated has to be recognized.

According to NCBI the HLA-allotypes with a frequency of 10% or more are distributed as it is shown in the following table.

TABLE

| | | | North | South-East |
|---|---|---|---|---|
| HLA- | Australian | European | American | Asian |
| allotype | frequency | frequency | frequency | frequency |
| HLA- | [%] | [%] | [%] | [%] |
| A*01:01 | | 16.4 | | |
| A*02:01 | 12.7 | 27.2 | 19.7 | |
| A*02:04 | | | | |
| A*03:01 | | 14.1 | | |
| A*11:01 | 13.5 | | | 20.4 |
| A*24:02 | 25.9 | | 37.7 | 29.9 |
| A*31:01:02 | | | | |
| A*34:01:01 | 40.1 | | | |
| B*07:02 | | 13.9 | | |
| B*08:01 | | 11.8 | | |
| B*13:01 | 23.8 | | | |
| B*15:04 | | | | 11.7 |
| B*15:21 | 10.6 | | | |
| B*44:02 | | 10.6 | | |
| B*56:01 | 16.1 | | | |
| B*56:02 | 10.3 | | | |
| C*01:02 | 24.6 | | | 13.3 |
| C*02:02 | | | 12.7 | |
| C*03:03 | | | | |
| C*03:04 | | | 20.4 | 17.3 |
| C*04:01 | 26.0 | 10.1 | 15.0 | |
| C*04:03 | 13.9 | | | |
| C*05:01 | | 10.6 | | |
| C*06:02 | | | | |
| C*07:01 | | 17.0 | | |
| C*07:02 | | 15.9 | 10.2 | 18.9 |
| C*08:01 | | | | 12.8 |
| C*15:02 | 16.5 | | | |

The term “frequency” denotes the frequency with which a specific HLA-allotype occurs within the entire human population. Thus, the term “with a relative frequency of 1% or more” denotes that the respective HLA-allotype has an occurrence within the entire human population of 1% or more. In one embodiment of all aspects the relative frequency is the relative frequency in the human population. In one embodiment the relative frequency is the relative frequency in the European population. In one embodiment the relative frequency is the relative frequency in the North-American population.

Thus, one aspect as reported herein is an antigen binding multi-function protein, characterized in that it comprises
one antigen presenting domain,
one antibody Fc-region, and
at least one antigen binding site,
wherein the antigen presenting domain comprises in N- to C-terminal direction
   either
      (i) a β2-microglobulin, and
      (ii) the extracellular domains α1, α2, and α3 of a class I MHC molecule with a relative frequency of less than 1%,
   or
      (i) the extracellular domains α1, α2, and α3 of a class I MHC molecule with a relative frequency of less than 1%, and
      (ii) a β2-microglobulin,
   or
      (i) a T-cell response eliciting peptide,
      (ii) a β2-microglobulin, and
      (iii) the extracellular domains α1, α2, and α3 of a class I MHC molecule with a relative frequency of 1% or more,
   or
      (i) a T-cell response eliciting peptide,
      (ii) the extracellular domains α1, α2, and α3 of a class I MHC molecule with a relative frequency of 1% or more, and
      (iii) a β2-microglobulin,
wherein the antigen binding site binds to a cancer cell surface antigen.

In one embodiment the antigen presenting domain that comprises in N- to C-terminal direction a β2-microglobulin and the extracellular domains α1, α2, and α3 of a class I MHC molecule with a relative frequency of less than 1% further comprises at its N-terminus a peptide binding to the MHC-peptide binding grove. In one embodiment the peptide is a T-cell-response eliciting peptide.

In one embodiment the T-cell-response eliciting peptide is a virus-derived peptide. In one embodiment the virus is selected from adenovirus, human herpesvirus 1, human herpesvirus 2, human herpesvirus 4 (Epstein-Barr virus), hepatitis-B-virus, hepatitis-C-virus, human cytomegalovirus, human immunodeficiency virus, human papillomavirus type 16, human papillomavirus type 18, human papillomavirus type 31, human papillomavirus type 33, human papillomavirus type 35, human papillomavirus type 39, human papillomavirus type 45, human papillomavirus type 51, human papillomavirus type 52, human papillomavirus type 56, human papillomavirus type 58, human papillomavirus type 59, human papillomavirus type 68, human papillomavirus type 73, human papillomavirus type 82, human T-cell lymphotropic virus type I, human influenza A virus, human influenza B virus, or vaccinia virus.

In one embodiment the virus-derived peptide is selected from NLVPMVATV (SEQ ID NO: 01), SLYNTVATL (SEQ ID NO: 48), GLCTLVAML (SEQ ID NO: 49), GILGFVFTL (SEQ ID NO: 50), STNRQSGRQ (SEQ ID NO: 51), LLFGYPVYV (SEQ ID NO: 52), FAEGFVRAL (SEQ ID NO: 53), LIVIGILIL (SEQ ID NO: 54), or ILHTPGCV (SEQ ID NO: 55).

In one embodiment the β2-microglobulin is human β2-microglobulin. In one embodiment the β2-microglobulin is consisting of the amino acid sequence of SEQ ID NO: 71.

In one embodiment the class I MHC molecule with a relative frequency of 1% or more is human HLA-A*0201. In one embodiment the extracellular domains α1, α2, and α3 of a class I MHC molecule is consisting of the amino acid sequence of SEQ ID NO: 72.

In one embodiment the virus-derived peptide is fused to the β2-microglobulin via a first linker peptide.

In one embodiment the β2-microglobulin is fused to the extracellular domain α1 of a class I MHC molecule via a second linker peptide.

In one embodiment the extracellular domain α3 of a class I MHC molecule is fused to the polypeptide (either disulfide-linked or not disulfide-linked) via a third linker peptide.

In one embodiment the first, second, and third linker peptide is the same or different.

In one embodiment the first linker peptide, the second linker peptide, and the third linker peptide are selected independently from each other from the amino acid sequences GS (SEQ ID NO: 73), GGS (SEQ ID NO: 74), GGGS (SEQ ID NO: 75), GGGSGGGS (SEQ ID NO: 76), GGGSGGGSGGGS (SEQ ID NO: 77), GGGSGGGSGGGSGGGS (SEQ ID NO: 78), GGGSGGGSGGGSGGGSGGGS (SEQ ID NO: 79), GGGGS (SEQ ID NO: 80), GGGGSGGGGS (SEQ ID NO: 81), GGGGSGGGGSGGGGS (SEQ ID NO: 82), GGGGSGGGGSGGGGSGGGGS (SEQ ID NO: 83), and GGGGSGGGGSGGGGSGGGGSGGGGS (SEQ ID NO: 84).

In one embodiment the first linker peptide comprises the amino acid sequence of SEQ ID NO: 82.

In one embodiment the second linker peptide comprises the amino acid sequence of SEQ ID NO: 83.

In one embodiment the third linker peptide comprises the amino acid sequence of SEQ ID NO: 73.

In one embodiment the antibody Fc-region is selected from an antibody Fc-region of a human antibody of the class IgG or the class IgE.

In one embodiment the antibody Fc-region is selected from an antibody Fc-region of a human antibody of the subclass IgG1, or IgG2, or IgG3, or IgG4.

In one embodiment the first disulfide-linked polypeptide and the second disulfide-linked polypeptide comprises a CH2 domain and a CH3 domain of human origin. In one embodiment the CH2 domain and the CH3 of human origin is of a human antibody of the class IgG or IgE. In one embodiment the CH2 domain and the CH3 domain is of a human antibody of the subclass IgG1, or IgG2, or IgG3, or IgG4. In one embodiment the CH2 domain comprises the amino acid sequence of SEQ ID NO: 85. In one embodiment the CH2 domain is of a human antibody of the subclass IgG1 or IgG2 and comprises at least one mutation of E233, L234, L235, G236, D265, D270, N297, E318, K320, K322, A327, P329, A330, and/or P331 (numbering according to the EU index of Kabat). In one embodiment the CH2 domain is of a human antibody of the subclass IgG1 or the human subclass IgG2 with the mutations L234A and L235A, and/or the mutations D265A and N297A, and/or contains the PVA236 mutation, and/or contains the mutation P329G. In one embodiment the CH2 domain is of a human antibody of the subclass IgG1 with the mutations L234A and L235A, and/or P329G. In one embodiment the CH2 domain is of a human antibody of the subclass IgG4 with the mutations S228P and/or L235E.

In one embodiment the first disulfide-linked polypeptide comprises the amino acid sequence of SEQ ID NO: 89 and the second disulfide-linked polypeptide comprises the amino acid sequence of SEQ ID NO: 90.

In one embodiment the first and the second disulfide-linked polypeptide comprise the amino acid sequence of SEQ ID NO: 94 or SEQ ID NO: 101.

In one embodiment the first disulfide-linked polypeptide or the second disulfide-linked polypeptide is consisting of the amino acid sequence of SEQ ID NO: 97 or SEQ ID NO: 98.

In one embodiment the first disulfide-linked polypeptide comprises the amino acid sequence of SEQ ID NO: 102 and the second disulfide-linked polypeptide comprises the amino acid sequence of SEQ ID NO: 103.

In one embodiment the disulfide-linked polypeptides are linked by two, or three, or four disulfide bonds.

In one embodiment the antigen presenting domain is characterized in that it comprises in N- to C-terminal direction
(i) a virus-derived peptide that has an amino acid sequence of SEQ ID NO: 01,
(ii) a first linker peptide that has an amino acid sequence of SEQ ID NO: 82.
(iii) a β2-microglobulin that has an amino acid sequence of SEQ ID NO: 71,
(iv) a second linker peptide that has an amino acid sequence of SEQ ID NO: 83,
(v) the extracellular domains α1, α2, and α3 of a class I MHC molecule that has an amino acid sequence of SEQ ID NO: 72, and
(vi) a third linker peptide that has an amino acid sequence of SEQ ID NO: 73.

From FIG. 10 it can be seen that the multi-function protein as reported herein maintains the binding properties of the antibody to which it is fused (FIG. 10 b) and c)).

In FIGS. 11 and 13 the in vitro efficacy and specificity of a multi-function protein as reported herein is shown.

The cytotoxicity assay was performed in the presence of CMV-specific CD8⁺ T-cells. It can be seen that a multi-function protein comprising a CMV-derived virus peptide induce the lysis/removal/disintegration of the target cells (see FIG. 11 a) for monovalent antibody, FIG. 11 b) for bivalent antibody). It can further be seen that the lysis of the target cells is highly specific as the incubation with the multi-function protein comprising an EBV-derived viral peptide (FIG. 11 b)) and control antibodies (FIG. 11 d) and e)) do not result in extensive cell lysis (the spontaneous lysis is about 3.5%).

In FIG. 13 the lysis of IGF-1R positive lung adenocarcinoma cell line H460M2 is shown.

The EC₅₀ value for a multi-function protein comprising a CMV-derived peptide and a bivalent antibody is about 10 ng/ml corresponding to about 50 pM. The determined EC₅₀ value is independent from the target cell to effector cell ratio (see FIG. 12; target cell to effector cell ratio from 1:3 to 1:1 corresponding to an effective ratio of 1:0.44 to 1:0.14 (76% of effector cells are CD8 positive and 19% are CMV specific)).

1. Affinity

In certain embodiments, a multi-function protein as provided herein comprises an antigen binding site derived from an antibody, e.g. a pair of antibody variable domains or a single domain antibody. In certain embodiments the antigen binding site has a dissociation constant (Kd) of ≦10 nM, ≦1 nM, ≦0.1 nM, ≦0.01 nM, or ≦0.001 nM (e.g. 10⁻⁸ M or less, e.g. from 10⁻⁸ M to 10⁻¹³ M, e.g., from 10⁻⁹ M to 10⁻¹³ M) with respect to its antigen.

In one embodiment, Kd is measured using surface plasmon resonance assays.

For example this can be done by using a BIACORE®-2000 or a BIACORE®-3000 instrument (BIAcore, Inc., Piscataway, N.J.) at 25° C. with immobilized antigen CMS chips at ˜10 response units (RU). Briefly, carboxymethylated dextran biosensor chips (CMS, BIAcore, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20) surfactant (PBST) at 25° C. at a flow rate of approximately 25 μl/min. Association rates (kon) and dissociation rates (koff) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio koff/kon. See, e.g., Chen, Y., et al., J. Mol. Biol. 293 (1999) 865-881.

2. Expression

Expression of specific formats of the multi-function protein as reported herein (different linker, different combinations of HLA and β2-microglobulin) in HEK 293 and CHO cells led to an accumulation of the multi-function protein, if detectable at all, within the endoplasmatic reticulum, i.e. isolation and secretion of the multi-function protein was strongly impaired.

No secretion of a multi-function protein to the cultivation medium could be detected when the multi-function protein was intended to comprise one of the polypeptides as outlined in the following tables.

TABLE.

| | | | | | | | |
|---|---|---|---|---|---|---|---|
| signal | CMV-derived | β2- | α1-α2-α3- | IgG-Fc-region | scFv | | |
| peptide | peptide | microglobulin | chains | | | | |
| signal | CMV-derived | β2- | α1-α2-α3- | antibody | | | |
| peptide | peptide | microglobulin | chains | heavy chain | | | |
| signal | CMV-derived | G₄S(G₃S)₂- | β2- | (G₄S)₃-linker | α1-α2-α3- | (G₄S)₂-linker | antibody |
| peptide | peptide | linker | microglobulin | | chains | | light chain |
| signal | β2-micro- | (G₄S)₃-linker | α1-α2-α3- | (G₄S)₂-linker | antibody | | |
| peptide | globulin | | chains | | light chain | | |
| signal | CMV-derived | (G₃S)₂GG- | α1-α2-α3- | (G₄S)₃-linker | β2-micro- | (G₄S)₂-linker | antibody |
| peptide | peptide | linker | chains | | globulin | | light chain |
| signal | CMV-derived | GGPGGGSG | α1-α2-α3- | (G₄S)₃-linker | β2-micro- | (G₄S)₂-linker | antibody |
| peptide | peptide | GG-linker | chains | | globulin | | light chain |
| signal | α1-α2-α3- | (G₄S)₃-linker | β2-micro- | (G₄S)₂-linker | antibody | | |
| peptide | chains | | globulin | | light chain | | |
| signal | CMV-derived | (G₃S)₂GG- | α1-α2-α3- | (G₄S)₃-linker | antibody | | |
| peptide | peptide | linker | chains | | light chain | | |
| signal | CMV-derived | GGPGGGSG | α1-α2-α3- | (G₄S)₃-linker | antibody | | |
| peptide | peptide | GG-linker | chains | | light chain | | |
| signal | CMV-derived | (G₃S)₂GG- | α1-α2-α3- | (G₄S)₃-linker | β2-micro- | (G₄S)₂-linker | antibody |
| peptide | peptide | linker | chains | | globulin | | heavy chain |

It has been found that the expression, and especially the secretion, of multi-function proteins comprising two antigen presenting domains formed of a virus-derived peptide linked to a MHC class I protein complex, and at least one variable domain and one constant domain of an antibody is not possible in eukaryotic cells.

Further it has been found that the expression, and especially the secretion, of multi-function proteins comprising two antigen presenting domains formed of a virus-derived peptide linked to a MHC class I protein multi-function protein, at least one variable domain, and an antibody hinge region is not possible in eukaryotic cells.

Thus, in a multi-function protein as reported herein an antigen presenting domain comprising a virus-derived peptide linked to a MHC class I protein cannot be present more than once and at least one antibody variable domain and one antibody constant domain has to be present in order to allow for the production and the secretion of the multi-function protein using eukaryotic cells.

Thus, a multi-function protein comprising exactly one antigen presenting domain of a virus-derived peptide linked to a MHC class I protein, an antibody heavy chain hinge region, and at least one antibody variable domain and one antibody constant domain can be recombinantly produced in and secreted from eukaryotic cells.

Thus, a multi-function protein comprising an antibody heavy chain hinge region, at least one pair of antibody variable domains, optionally an antibody constant domain, and exactly one antigen presenting domain of a virus-derived peptide linked to a MHC class I protein can be recombinantly produced in and secreted from eukaryotic cells.

Various combinations were tested. Secreted expression of multi-function proteins can be accomplished by e.g. N-terminal fusion of an immunoglobulin-derived signal peptide wherein the virus-derived peptide is fused N-terminally to the class I MHC molecule. Class I MHC molecule heavy chain (α1-α2-α3 lacking the transmembrane and the cytoplasmatic domain) and light chain (β2-microglobulin) can be changed in order. The different antigen presenting domains were N-terminally fused to either an antibody light chain or an antibody heavy chain hinge region comprising polypeptide. Exemplary combinations are shown in FIG. 2.

As can be seen from the following table multi-function proteins comprising antigen presenting domains containing an MHC-I protein complex can only be expressed in the presence of variable antibody domain and antibody hinge region derived polypeptides when a single viral-derived-peptide-microglobulin-HLA-fusion polypeptide is present.

TABLE

| | | | | contains | | |
|---|---|---|---|---|---|---|
| | number of | | | antibody | | |
| | virus-derived | number | number | heavy chain | | |
| | peptide-class I | of | of | hinge region | | |
| | MHC fusion | variable | constant | comprising | expression | lane in |
| | polypeptide | domains | domains | polypeptide | Level | FIG. 3 |
| 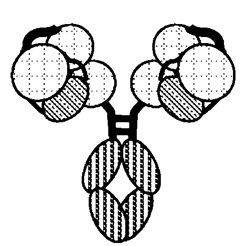 | 2 | 0 | 0 | yes | high | 1 |
| 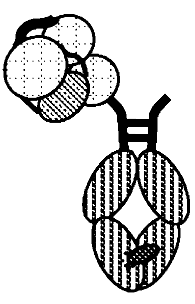 | 1 | 0 | 0 | yes | high | 2 |
| 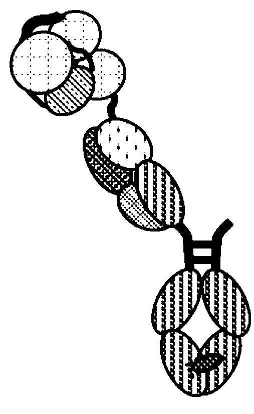 | 1 | 1 | 1 | yes | high | 3 |
| 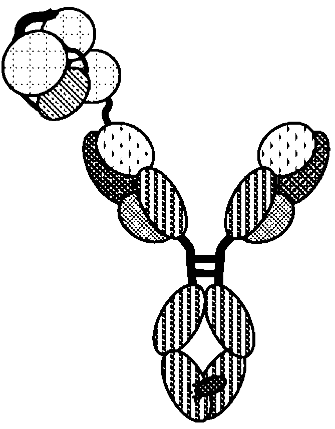 | 1 | 2 | 2 | yes | high | 4 |
| 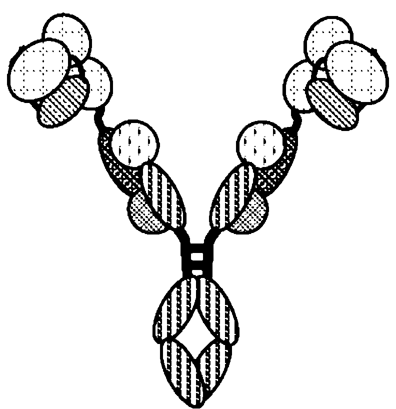 | 2 | 2 | 2 | yes | no expression | 5 |
| 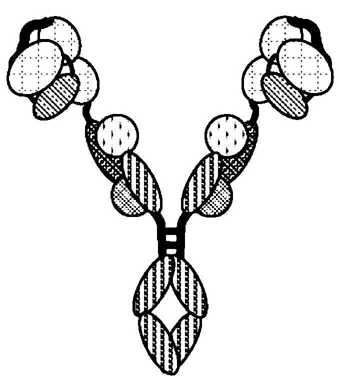 | 2 | 2 | 2 | yes | no expression | 6 |
| 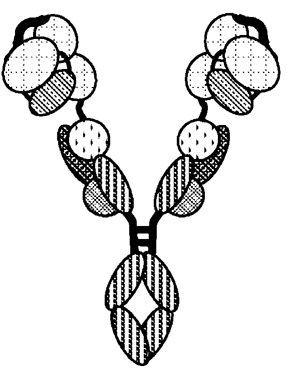 | 2 | 2 | 2 | yes | very low | 7 |
| 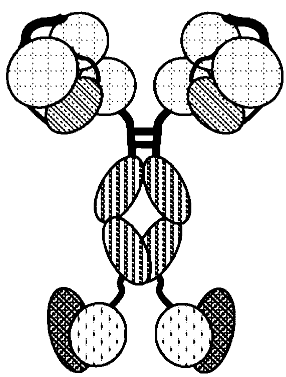 | 2 | 2 | 0 | yes | no expression | 8 |
| 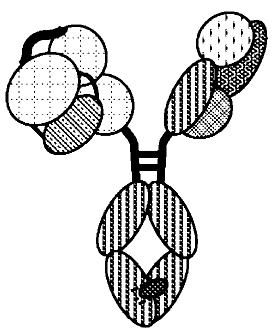 | 1 | 1 | 1 | yes | high | 9 |

In some embodiments the multi-function protein as reported herein comprises different pairs of polypeptides. In order to allow proper pairing of the polypeptides the knobs-into-holes technology or the cross-mAb technology can be used in order to reduce the amount of not correctly associated multi-function protein.

The knob modification denotes the mutation T366W in the CH3 domain of an antibody (numbering according to EU index of Kabat).

The hole-modification denotes the mutations T366S, L368A and Y407V in the CH3 domain of an antibody (numbering according to EU index of Kabat).

In addition to the knob and hole modification the mutation S354C in the one CH3 domain and the mutation Y349C in the other CH3 domain can be present.

The cross-mAb technology is reported e.g. in WO 2009/080251, WO 2009/080252, WO 2009/080254, WO 2009/080253, WO 2010/112193, WO 2010/115589, WO 2010/136172, WO 2010/145792, and WO 2010/145793.

3. Variants

In certain embodiments, amino acid sequence variants of the multi-function protein provided herein are contemplated. For example, it may be desirable to improve the binding affinity and/or other biological properties of the multi-function protein. Amino acid sequence variants of the multi-function protein may be prepared by introducing appropriate modifications into the nucleotide sequence encoding the polypeptide chains of the multi-function protein, or by peptide synthesis. Such modifications include, for example, deletions from, and/or insertions into and/or substitutions of residues within the amino acid sequences of the polypeptides of the multi-function protein. Any combination of deletion, insertion, and substitution can be made to arrive at the final construct, provided that the final construct possesses the desired characteristics, e.g., antigen-binding.

a) Substitution, Insertion, and Deletion Variants

In certain embodiments, multi-function protein variants having one or more amino acid substitutions in one or more of the polypeptide chains are provided. Exemplary changes are provided in the following table under the heading of “exemplary substitutions”, and as further described below in reference to amino acid side chain classes. Conservative substitutions are shown in the following Table under the heading of “preferred substitutions”. Amino acid substitutions may be introduced into a multi-function protein of interest and the products screened for a desired activity, e.g., retained/improved antigen binding, decreased immunogenicity, or improved ADCC or CDC.

TABLE

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include a multi-function protein comprising a polypeptide with an N-terminal methionyl residue. Other insertional variants include the fusion to the N- or C-terminus of the polypeptide chains of the multi-function protein to an enzyme.

b) Glycosylation Variants

In certain embodiments, one or more polypeptides of the multi-function protein provided herein can be altered to increase or decrease the extent to which the polypeptide(s) is(are) glycosylated. Addition or deletion of glycosylation sites to a polypeptide may be conveniently accomplished by altering the amino acid sequence such that one or more glycosylation sites is created or removed.

The multi-function protein comprises an antibody Fc-region and the carbohydrate attached thereto may be altered. Native Fc-regions produced by mammalian cells typically comprise a branched, biantennary oligosaccharide that is generally attached by an N-linkage to Asn297 of the CH2 domain of the Fc-region. See, e.g., Wright, A., and Morrison, S. L., TIBTECH 15 (1997) 26-32. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in a multi-function protein as reported herein may be made in order to create variants with certain improved properties.

In one embodiment, multi-function protein comprising polypeptide variants are provided having a carbohydrate structure that lacks fucose attached (directly or indirectly) to the Fc-region. For example, the amount of fucose in such Fc-region may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. multi-function protein, hybrid and high mannose structures) as measured by MALDI-TOF mass spectrometry, as described in WO 2008/077546, for example. Asn297 refers to the asparagine residue located at about position 297 in the Fc-region (EU numbering of Fc-region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US 2003/0157108; US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki, A., et al., J. Mol. Biol. 336 (2004) 1239-1249; Yamane-Ohnuki, N., et al., Biotech. Bioeng. 87 (2004) 614-622. Examples of cell lines capable of producing defucosylated antibodies include Lec13 CHO cells deficient in protein fucosylation (Ripka, J., et al., Arch. Biochem. Biophys. 249 (1986) 533-545; US 2003/0157108; and WO 2004/056312, especially at Example 11), and knockout cell lines, such as alpha-1,6-fucosyltransferase gene, FUT8, knockout CHO cells (see, e.g., Yamane-Ohnuki, N., et al., Biotech. Bioeng. 87 (2004) 614-622; Kanda, Y., et al., Biotechnol. Bioeng. 94 (2006) 680-688; and WO 2003/085107).

Multi-function proteins comprising Fc-region variants are further provided with bisected oligosaccharides, e.g., in which a biantennary oligosaccharide attached to the Fc-region is bisected by GlcNAc. Such variants may have reduced fucosylation and/or improved ADCC function. Examples of such antibody variants are described, e.g., in WO 2003/011878; U.S. Pat. No. 6,602,684; and US 2005/0123546. Fc-region variants with at least one galactose residue in the oligosaccharide attached to the Fc-region are also provided. Such Fc-region variants may have improved CDC function. Corresponding antibody variants are described, e.g., in WO 97/30087; WO 98/58964; and WO 99/22764.

c) Fc-Region Variants

In certain embodiments, one or more amino acid modifications may be introduced into the Fc-region of the multi-function protein provided herein, thereby generating an Fc-region variant. The Fc-region variant may comprise a human Fc-region sequence (e.g., a human IgG1, IgG2, IgG3 or IgG4 Fc-region) comprising an amino acid modification (e.g. a substitution) at one or more amino acid positions.

In certain embodiments, the invention contemplates an Fc-region variant that possesses some but not all effector functions, which make it a desirable candidate for applications in which the half-life of the multi-function protein in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the multi-function protein lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch, J. V., and Kinet, J. P., Annu. Rev. Immunol. 9 (1991) 457-492. Non-limiting examples of in vitro assays to assess ADCC activity of a molecule of interest is described in U.S. Pat. No. 5,500,362 (see, e.g. Hellstrom, I., et al., Proc. Natl. Acad. Sci. USA 83 (1986) 7059-7063; and Hellstrom, I., et al., Proc. Natl. Acad. Sci. USA 82 (1985) 1499-1502); U.S. Pat. No. 5,821,337 (see Bruggemann, M., et al., J. Exp. Med. 166 (1987) 1351-1361). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes, R., et al., Proc. Natl. Acad. Sci. USA 95 (1998) 652-656. C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro, H., et al., J. Immunol. Methods 202 (1996) 163-171; Cragg, M. S., et al., Blood 101 (2003) 1045-1052; and Cragg, M. S. and Glennie, M. J., Blood 103 (2004) 2738-2743). FcRn binding and in vivo clearance/half-life determinations can also be performed using methods known in the art (see, e.g., Petkova, S. B., et al., Int. Immunol. 18 (2006) 1759-1769).

Fc-regions with reduced effector function include those with substitution of one or more of Fc-region residues 234, 235, 238, 265, 269, 270, 297, 327 and 329 (see e.g. U.S. Pat. No. 6,737,056). Such Fc-region mutants include Fc-region mutants with substitutions at two or more of amino acid positions 265, 269, 270, 297 and 327, including the so-called “DANA” Fc-region mutant with substitution of residues 265 and 297 to alanine (U.S. Pat. No. 7,332,581).

Certain Fc-region variants with improved or diminished binding to FcRs are described. (See, e.g., U.S. Pat. No. 6,737,056; WO 2004/056312, and Shields, R. L., et al., J. Biol. Chem. 276 (2001) 6591-6604).

In certain embodiments, a multi-function protein variant comprises an Fc-region with one or more amino acid substitutions which improve ADCC, e.g., substitutions at positions 298, 333, and/or 334 of the Fc-region (EU numbering of residues).

In some embodiments, alterations are made in the Fc-region that result in altered (i.e., either improved or diminished) C1q binding and/or Complement Dependent Cytotoxicity (CDC), e.g., as described in U.S. Pat. No. 6,194,551, WO 99/51642, and Idusogie, E. E., et al., J. Immunol. 164 (2000) 4178-4184.

Antibodies with increased half-lives and improved binding to the neonatal Fc receptor (FcRn), which is responsible for the transfer of maternal IgGs to the fetus (Guyer, R. L., et al., J. Immunol. 117 (1976) 587-593, and Kim, J. K., et al., J. Immunol. 24 (1994) 2429-2434), are described in US 2005/0014934. Those antibodies comprise an Fc-region with one or more substitutions therein which improve binding of the Fc-region to FcRn. Such Fc-region variants include those with substitutions at one or more of Fc-region residues: 238, 256, 265, 272, 286, 303, 305, 307, 311, 312, 317, 340, 356, 360, 362, 376, 378, 380, 382, 413, 424 or 434, e.g., substitution of Fc-region residue 434 (U.S. Pat. No. 7,371,826).

See also Duncan, A. R. and Winter, G., Nature 322 (1988) 738-740; U.S. Pat. No. 5,648,260; U.S. Pat. No. 5,624,821; and WO 94/29351 concerning other examples of Fc-region variants.

B. Recombinant Methods and Compositions

Multi-function proteins as reported herein may be produced using recombinant methods and compositions, e.g., as described in U.S. Pat. No. 4,816,567. In one embodiment, isolated nucleic acids encoding the polypeptides of the multi-function protein described herein are provided. In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp2/0 cell). In one embodiment, a method of making a multi-function protein as reported herein is provided, wherein the method comprises culturing a host cell comprising a nucleic acid encoding the polypeptides of the multi-function protein, as provided above, under conditions suitable for expression of the polypeptides and formation of the multi-function protein, and optionally recovering the multi-function protein from the host cell (or host cell culture medium).

For recombinant production of a multi-function protein, nucleic acid encoding the polypeptides of the multi-function protein, e.g., as described above, are isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression vectors include prokaryotic or eukaryotic cells described herein. For example, multi-function proteins may be produced in bacteria, in particular when glycosylation and Fc effector function are not needed. For expression of antibody fragments and polypeptides in bacteria, see, e.g., U.S. Pat. No. 5,648,237, U.S. Pat. No. 5,789,199, and U.S. Pat. No. 5,840,523. (See also Charlton, K. A., In: Methods in Molecular Biology, Vol. 248, Lo, B. K. C. (ed.), Humana Press, Totowa, N.J. (2003), pp. 245-254, describing expression of antibody fragments in E. coli.) After expression, the multi-function protein may be isolated from the bacterial cell paste in a soluble fraction and can be further purified.

In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of an antibody with a partially or fully human glycosylation pattern. See Gerngross, T. U., Nat. Biotech. 22 (2004) 1409-1414; and Li, H., et al., Nat. Biotech. 24 (2006) 210-215.

Suitable host cells for the expression of glycosylated multi-function proteins are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. No. 5,959,177, U.S. Pat. No. 6,040,498, U.S. Pat. No. 6,420,548, U.S. Pat. No. 7,125,978, and U.S. Pat. No. 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (HEK 293 or 293 cells as described, e.g., in Graham, F. L., et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather, J. P., et al., Annals N.Y. Acad. Sci. 383 (1982) 44-68; MRC 5 cells; and FS4 cells. Other useful mammalian host cell lines include Chinese hamster ovary (CHO) cells, including DHFR⁻ CHO cells (Urlaub, G., et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, N.J. (2004), pp. 255-268.

C. Pharmaceutical Formulations

Pharmaceutical formulations of a multi-function protein as described herein are prepared by mixing such multi-function protein having the desired degree of purity with one or more optional pharmaceutically acceptable carriers (Remington's Pharmaceutical Sciences, 16th edition, Osol, A. (ed.) (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyl dimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as poly(vinylpyrrolidone); amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rhuPH20 (HYLENEX®, Baxter International, Inc.). Certain exemplary sHASEGPs and methods of use, including rhuPH20, are described in US 2005/0260186 and US 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methyl methacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. (ed.) (1980).

Sustained-release preparations may be prepared. Suitable examples of sustained-release preparations include semi-permeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

D. Therapeutic Methods and Compositions

Any of the multi-function proteins provided herein may be used in therapeutic methods.

In one aspect, a multi-function protein as reported herein for use as a medicament is provided.

In further aspects, a multi-function protein as reported herein for use in treating cancer is provided.

In certain embodiments, a multi-function protein as reported herein for use in a method of treatment is provided.

In certain embodiments, the invention provides a multi-function protein as reported herein for use in a method of treating an individual having cancer comprising administering to the individual an effective amount of the multi-function protein as reported herein. In one such embodiment, the method further comprises administering to the individual an effective amount of at least one additional therapeutic agent, e.g., as described below.

In further embodiments, the invention provides a multi-function protein as reported herein for use in removal of cancer cells. In certain embodiments, the invention provides a multi-function protein as reported herein for use in a method of removal of cancer cells in an individual comprising administering to the individual an effective of the multi-function protein as reported herein to remove cancer cells. An “individual” according to any of the above embodiments may be a human.

In a further aspect, the invention provides for the use of a multi-function protein as reported herein in the manufacture or preparation of a medicament. In one embodiment, the medicament is for treatment of cancer. In a further embodiment, the medicament is for use in a method of treating cancer comprising administering to an individual having cancer an effective amount of the medicament. In one such embodiment, the method further comprises administering to the individual an effective amount of at least one additional therapeutic agent. In a further embodiment, the medicament is for the removal of cancer cells. In a further embodiment, the medicament is for use in a method of removal of cancer cells in an individual comprising administering to the individual an amount effective of the medicament to remove cancer cells. An “individual” according to any of the above embodiments may be a human.

In a further aspect, the invention provides a method for treating cancer. In one embodiment, the method comprises administering to an individual having such cancer an effective amount of a multi-function protein as reported herein. In one such embodiment, the method further comprises administering to the individual an effective amount of at least one additional therapeutic agent. An “individual” according to any of the above embodiments may be a human.

In a further aspect, the invention provides a method for removal of cancer cells in an individual. In one embodiment, the method comprises administering to the individual an effective amount of a multi-function protein as reported herein to remove cancer cells. In one embodiment, an “individual” is a human.

In a further aspect, the invention provides pharmaceutical formulations comprising any of the multi-function proteins as reported herein, e.g., for use in any of the above therapeutic methods. In one embodiment, a pharmaceutical formulation comprises any of the multi-function proteins as reported herein and a pharmaceutically acceptable carrier. In another embodiment, a pharmaceutical formulation comprises any of the multi-function proteins as reported herein and at least one additional therapeutic agent.

Multi-function proteins of the invention can be used either alone or in combination with other agents in a therapy. For instance, a multi-function protein of the invention may be co-administered with at least one additional therapeutic agent.

Such combination therapies noted above encompass combined administration (where two or more therapeutic agents are included in the same or separate formulations), and separate administration, in which case, administration of the multi-function protein of the invention can occur prior to, simultaneously, and/or following, administration of the additional therapeutic agent and/or adjuvant. Multi-function proteins of the invention can also be used in combination with radiation therapy.

A multi-function protein of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Multi-function proteins of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The multi-function protein need not be, but is optionally formulated with one or more agents currently used to prevent or treat the disorder in question. The effective amount of such other agents depends on the amount of multi-function protein present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a multi-function protein of the invention (when used alone or in combination with one or more other additional therapeutic agents) will depend on the type of disease to be treated, the type of multi-function protein, the severity and course of the disease, whether the multi-function protein is administered for preventive or therapeutic purposes, previous therapy, the patient's clinical history and response to the multi-function protein, and the discretion of the attending physician. The multi-function protein is suitably administered to the patient at one time or over a series of treatments. Depending on the type and severity of the disease, about 1 μg/kg to 15 mg/kg (e.g. 0.5 mg/kg-10 mg/kg) of multi-function protein can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

It is understood that any of the above formulations or therapeutic methods may be carried out using an immunoconjugate of the invention in place of or in addition to a multi-function protein as reported herein.

One aspect as reported herein is the multi-function protein as reported herein for use in a method of treating a cancer in a patient, wherein the multi-function protein is to be administered before, simultaneously or after the immunization of the patient with the virus-derived peptide comprised in the multi-function protein.

One aspect as reported herein is the use of a multi-function protein as reported herein for the manufacture of a medicament for the treatment of cancer in combination with immunization against the virus-derived peptide comprised in the multi-function protein.

In the first step the virus-derived peptide as contained in the multi-function protein is administered first to the individual to be treated. At a certain time span later, i.e. between 4 days and 28 days, the multi-function protein as reported herein is administered to the individual.

By this successive and separated application of the virus-derived polypeptide, in the first step alone and in the second step in the multi-function protein as reported herein, it is possible to increase the number of virus-derived peptide specific T-cell and, thus, to increase the efficacy of the treatment.

### III. ARTICLES OF MANUFACTURE

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a multi-function protein of the invention. The label or package insert indicates that the composition is used for treating the condition of choice. Moreover, the article of manufacture may comprise (a) a first container with a composition contained therein, wherein the composition comprises a multi-function protein of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to a multi-function protein as reported herein.

### IV. SPECIFIC EMBODIMENTS

1. A multi-function protein, characterized in that it comprises
   (exactly) one antigen presenting domain,
   (exactly) one antibody Fc-region, and
   at least one antigen binding site,
   wherein the antigen presenting domain comprises in N- to C-terminal direction
      either
         (i) a β2-microglobulin, and
         (ii) the extracellular domains α1, α2, and α3 of a class I MHC molecule with a relative frequency of less than 1%,
      or
         (i) the extracellular domains α1, α2, and α3 of a class I MHC molecule with a relative frequency of less than 1%, and
         (ii) a β2-microglobulin,
      or
         (i) a T-cell response eliciting peptide,
         (ii) a β2-microglobulin, and
         (iii) the extracellular domains α1, α2, and α3 of a class I MHC molecule with a relative frequency of 1% or more,
      or
         (i) a T-cell response eliciting peptide,
         (ii) the extracellular domains α1, α2, and α3 of a class I MHC molecule with a relative frequency of 1% or more, and
         (iii) a β2-microglobulin,
   wherein the antigen binding site binds to a cancer cell surface antigen.
2. The multi-function protein according to item 1, characterized in that the antibody Fc-region comprises a first and second disulfide-linked Fc-region polypeptide, whereby the antigen binding site comprises the first Fc-region polypeptide.
3. The multi-function protein according to any one of items 1 to 2, characterized in that the antigen binding site comprises i) a pair of an antibody heavy chain and an antibody light chain, or ii) a scFv fusion polypeptide comprising in N- to C-terminal direction a scFv antibody fragment and an antibody Fc-region polypeptide, or iii) a scFab fusion polypeptide comprising in N- to C-terminal direction a scFab and an antibody Fc-region polypeptide.
4. The multi-function protein according to any one of items 1 to 3, characterized in that i) the antigen presenting domain is linked to the N-terminus of the heavy chain or to the N-terminus of the light chain of the antigen binding site, or ii) the antigen presenting domain is linked to the C-terminus of the heavy chain or to the C-terminus of the light chain of the antigen binding site, or iii) the antigen presenting domain is linked to the N- or C-terminus of the scFv fusion polypeptide, or iv) the antigen presenting domain is linked to the N- or C-terminus of the scFab fusion polypeptide, or iv) the antigen presenting domain is linked to the N- or C-terminus of the second Fc-region polypeptide.
5. The multi-function protein according to any one of items 1 to 4, characterized in that the cancer cell surface antigen is melanoma-associated chondroitin sulfate proteoglycan (MCSP).
6. The multi-function protein according to any one of items 1 to 5, characterized in that the T-cell response eliciting peptide is a virus-derived peptide.
7. The multi-function protein according to any one of items 1 to 6, characterized in that the T-cell response eliciting peptide is a CD8⁺-T-cell response eliciting peptide.
8. The multi-function protein according to any one of items 6 to 7, characterized in that the virus-derived peptide is a human cytomegalovirus-derived peptide.
9. The multi-function protein according to any one of items 1 to 8, characterized in that the virus-derived peptide has an amino acid sequence selected from the group of SEQ ID NO: 01 to SEQ ID NO: 70.
10. The multi-function protein according to any one of items 1 to 9, characterized in that the virus-derived peptide has the amino acid sequence of SEQ ID NO: 01.
11. The multi-function protein according to any one of items 1 to 10, characterized in that the class I MHC molecule with a relative frequency of 1% or more is selected from the group comprising HLA-A*0201, HLA-A*1101, HLA-A*2402, HLA-A*340101, HLA-C*0304, HLA-C*0401, and HLA-C*0702.
12. The multi-function protein according to any one of items 1 to 11, characterized in that the class I MHC molecule with a relative frequency of 1% or more is selected depending on the region of the individual to whom the multi-function protein is to be administered as follows:
   for an individual of European origin the class I MHC molecule is selected from the group comprising HLA-A*0101, HLA-A*0201, HLA-A*0301, HLA-B*0702, HLA-B*0801, HLA-B*4402, HLA-C*0401, HLA-C*0501, HLA-C*0701, and HLA-C*0702,
   for an individual of Australian origin the class I MHC molecule is selected from the group comprising HLA-A*0201, HLA-A*1101, HLA-A*2402, HLA-A*340101, HLA-B*1301, HLA-B*1521, HLA-B*5601, HLA-B*5602, HLA-C*0102, HLA-C*0401, HLA-C*0403, and HLA-C*1502,
   for an individual of North American origin the class I MHC molecule is selected from the group comprising HLA-A*0201, HLA-A*2402, HLA-C*0202, HLA-C*0304, HLA-C*0401, and HLA-C*0702, and
   for an individual of South-East-Asian origin the class I MHC molecule is selected from the group comprising HLA-A*1101, HLA-A*2402, HLA-B*1504, HLA-C*0102, HLA-C*0304, HLA-C*0702, and HLA-C*0801.
13. The multi-function protein according to any one of items 1 to 12, characterized in that the class I MHC molecule with a relative frequency of 1% or more is selected depending on the region of the individual to whom the multi-function protein is to be administered as follows:
   for an individual of European origin the class I MHC molecule is HLA-A*0201,
   for an individual of Australian origin the class I MHC molecule is selected from the group comprising HLA-A*2402, HLA-B*1301, HLA-C*0102, and HLA-C*0401,
   for an individual of North American origin the class I MHC molecule is selected from the group comprising HLA-A*2402, and HLA-C*0304, and
   for an individual of South-East-Asian origin the class I MHC molecule is HLA-A*2402.
14. The multi-function protein according to any one of items 1 to 13, characterized in that the class I MHC molecule with a relative frequency of less than 1% is selected from the group comprising HLA-B*4201, HLA-B*5901, HLA-B*6701, and HLA-B*7802.
15. The multi-function protein according to any one of items 1 to 14, characterized in that the β2-microglobulin is human β2-microglobulin and the class I MHC molecule with a relative frequency of 10% or more is human HLA-A*0201.
16. The multi-function protein according to any one of items 1 to 15, characterized in that the antigen presenting domain comprises
   (i) a virus-derived peptide,
   (ii) β2-microglobulin, and
   (iii) the soluble HLA-A allele A*0201
   or
   (i) a virus-derived peptide,
   (ii) the soluble HLA-A allele A*0201, and
   (iii) β2-microglobulin.
17. The multi-function protein according to any one of items 1 to 16, characterized in that the β2-microglobulin is consisting of the amino acid sequence of SEQ ID NO: 71 or is a variant thereof comprising of from 1 to 10 amino acid exchanges, additions, and/or deletions.
18. The multi-function protein according to any one of items 1 to 17, characterized in that the extracellular domains α1, α2 and α3 of a class I MHC molecule is consisting of the amino acid sequence of SEQ ID NO: 72 or is a variant thereof comprising of from 1 to 10 amino acid exchanges, additions, and/or deletions.
19. The multi-function protein according to any one of items 1 to 18, characterized in that the antigen presenting domain comprises in N- to C-terminal direction
   (i) a virus-derived peptide that has an amino acid sequence selected from the group comprising SEQ ID NO: 01 to SEQ ID NO: 70,
   (ii) a first linker peptide that has an amino acid sequence selected from the group comprising SEQ ID NO: 77, 78, 79, 82, 83, and 84,
   (iii) a β2-microglobulin that has an amino acid sequence of SEQ ID NO: 71,
   (iv) a second linker peptide that has an amino acid sequence selected from the group comprising SEQ ID NO: 77, 78, 79, 82, 83, and 84,
   (v) the extracellular domains α1, α2, and α3 of a class I MHC molecule that has an amino acid sequence of SEQ ID NO: 72, and
   (vi) a third linker peptide that has an amino acid sequence selected from the group comprising SEQ ID NO: 73, 77, 78, 79, 82, 83, 84, and 136.
20. The multi-function protein according to any one of items 1 to 19, characterized in that the multi-function protein is characterized in that the antigen presenting domain comprises in N- to C-terminal direction
   (i) a virus-derived peptide that has an amino acid sequence selected from the group comprising SEQ ID NO: 01 to SEQ ID NO: 70,
   (ii) a first linker peptide that has an amino acid sequence selected from the group comprising SEQ ID NO: 77, 78, 79, 82, 83, and 84,
   (iii) a β2-microglobulin that has an amino acid sequence of SEQ ID NO: 71 or is a variant thereof comprising of from 1 to 10 amino acid exchanges, additions, and/or deletions,
   (iv) a second linker peptide that has an amino acid sequence selected from the group comprising SEQ ID NO: 77, 78, 79, 82, 83, and 84,
   (v) the extracellular domains α1, α2 and α3 of a class I MHC molecule that has an amino acid sequence of SEQ ID NO: 72 or is a variant thereof comprising of from 1 to 10 amino acid exchanges, additions, and/or deletions, and
   (vi) a third linker peptide that has an amino acid sequence selected from the group comprising SEQ ID NO: 73, 77, 78, 79, 82, 83, 84, 136.
21. The multi-function protein according to item 20, characterized in that
   the first linker peptide has the amino acid sequence of SEQ ID NO: 82, and/or
   the second linker peptide has the amino acid sequence of SEQ ID NO: 83, and/or
   the third linker peptide has the amino acid sequence of SEQ ID NO: 136.
22. The multi-function protein according to any one of items 1 to 21, characterized in that the antibody Fc-region is selected from an antibody Fc-region of a human antibody of the class IgG or the class IgE.
23. The multi-function protein according to any one of items 1 to 22, characterized in that the antibody Fc-region is selected from an antibody Fc-region of a human antibody of the subclass IgG1, or IgG2, or IgG3, or IgG4.
24. The multi-function protein according to any one of items 1 to 23, characterized in that the antibody Fc-region is of a human antibody of the subclass IgG1 or IgG2 and comprises at least one mutation in E233, L234, L235, G236, D265, D270, N297, E318, K320, K322, A327, P329, A330, and/or P331 (numbering according to the EU index of Kabat).
25. The multi-function protein according to any one of items 1 to 24, characterized in that the antibody Fc-region is of a human antibody of the subclass IgG1 or the human subclass IgG2 with the mutations L234A and L235A, and/or the mutations D265A and N297A, and/or contains the PVA236 mutation, and/or contains the mutation P329G.
26. The multi-function protein according to any one of items 1 to 25, characterized in that the antibody Fc-region is of a human antibody of the subclass IgG1 with the mutations L234A and L235A and/or P329G.
27. The multi-function protein according to any one of items 1 to 25, characterized in that the antibody Fc-region is of a human antibody of the subclass IgG4 with the mutation S228P and/or L235E.
28. The multi-function protein according to any one of items 1 to 25, characterized in that the first and second antibody Fc-region polypeptide is selected independently of each other from the group comprising SEQ ID NO: 87 to 103.
29. The multi-function protein according to any one of items 1 to 25, characterized in that the antibody Fc-region comprises two Fc-region polypeptides with the amino acid sequence of SEQ ID NO: 94.
30. The multi-function protein according to any one of items 1 to 25, characterized in that the antibody Fc-region comprises two Fc-region polypeptides with the amino acid sequence of SEQ ID NO: 100.
31. The multi-function protein according to any one of items 1 to 25, characterized in that the antibody Fc-region comprises two Fc-region polypeptides with the amino acid sequence of SEQ ID NO: 101.
32. The multi-function protein according to any one of items 1 to 25, characterized in that the antibody Fc-region comprises a first Fc-region polypeptide with the amino acid sequence of SEQ ID NO: 89 and a second Fc-region polypeptide with the amino acid sequence of SEQ ID NO: 90.
33. The multi-function protein according to any one of items 1 to 25, characterized in that the antibody Fc-region comprises a first Fc-region polypeptide with the amino acid sequence of SEQ ID NO: 97 and a second Fc-region polypeptide with the amino acid sequence of SEQ ID NO: 98.
34. The multi-function protein according to any one of items 1 to 25, characterized in that the antibody Fc-region comprises a first Fc-region polypeptide with the amino acid sequence of SEQ ID NO: 102 and a second Fc-region polypeptide with the amino acid sequence of SEQ ID NO: 103.
35. A multi-function protein, characterized in that it comprises
   exactly one antigen presenting domain, which comprises in N- to C-terminal direction
      either
         (i) a β2-microglobulin, and
         (ii) the extracellular domains α1, α2, and α3 of a class I MHC molecule with a relative frequency of less than 1%,
      or
         (i) the extracellular domains α1, α2, and α3 of a class I MHC molecule with a relative frequency of less than 1%, and
         (ii) a β2-microglobulin,
      or
         (i) a T-cell response eliciting peptide,
         (ii) a β2-microglobulin, and
         (iii) the extracellular domains α1, α2, and α3 of a class I MHC molecule with a relative frequency of 1% or more,
      or
         (i) a T-cell response eliciting peptide,
         (ii) the extracellular domains α1, α2, and α3 of a class I MHC molecule with a relative frequency of 1% or more, and
         (iii) a β2-microglobulin,
   exactly one antibody Fc-region, and
   at least one antigen binding site, which specifically binds to melanoma-associated chondroitin sulfate proteoglycan (MCSP).
36. A multi-function protein, characterized in that it comprises
   exactly one antigen presenting domain, which comprises in N- to C-terminal direction
      (i) a T-cell response eliciting peptide,
      (ii) a β2-microglobulin, and
      (iii) the extracellular domains α1, α2, and α3 of a class I MHC molecule with a relative frequency of 1% or more,
   exactly one antibody Fc-region, and
   at least one antigen binding site, which specifically binds to melanoma-associated chondroitin sulfate proteoglycan (MCSP).
37. A multi-function protein, characterized in that it comprises
   exactly one antigen presenting domain, which comprises in N- to C-terminal direction
      (i) a T-cell response eliciting peptide of SEQ ID NO: 01,
      (ii) a β2-microglobulin of SEQ ID NO: 71, and
      (iii) the extracellular domains α1, α2, and α3 of a class I MHC molecule of SEQ ID NO: 72,
   exactly one antibody Fc-region, and
   at least one antigen binding site, which specifically binds to melanoma-associated chondroitin sulfate proteoglycan (MCSP).
38. A multi-function protein, characterized in that it comprises
   exactly one antigen presenting domain, which comprises in N- to C-terminal direction
      (i) a T-cell response eliciting peptide of SEQ ID NO: 01,
      (ii) a β2-microglobulin of SEQ ID NO: 71, and
      (iii) the extracellular domains α1, α2, and α3 of a class I MHC molecule of SEQ ID NO: 72,
   exactly one antibody Fc-region, which comprises two disulfide-linked Fc-region polypeptides, whereof the first disulfide-linked Fc-region polypeptide has the amino acid sequence of SEQ ID NO: 97 and the second disulfide-linked Fc-region polypeptide has the amino acid sequence of SEQ ID NO: 98, and
   at least one antigen binding site, which specifically binds to melanoma-associated chondroitin sulfate proteoglycan (MCSP).
39. A multi-function protein, characterized in that it comprises
   exactly one antigen presenting domain, which comprises in N- to C-terminal direction
      (i) a T-cell response eliciting peptide of SEQ ID NO: 01,
      (ii) a β2-microglobulin of SEQ ID NO: 71, and
      (iii) the extracellular domains α1, α2, and α3 of a class I MHC molecule of SEQ ID NO: 72,
   exactly one antibody Fc-region, which comprises two disulfide-linked Fc-region polypeptides, whereof the first disulfide-linked Fc-region polypeptide has the amino acid sequence of SEQ ID NO: 102 and the second disulfide-linked Fc-region polypeptide has the amino acid sequence of SEQ ID NO: 103, and
   at least one antigen binding site, which specifically binds to melanoma-associated chondroitin sulfate proteoglycan (MCSP).
40. A multi-function protein, characterized in that it comprises
   exactly one antigen presenting domain, which comprises in N- to C-terminal direction
      (i) a T-cell response eliciting peptide of SEQ ID NO: 01,
      (ii) a β2-microglobulin of SEQ ID NO: 71, and
      (iii) the extracellular domains α1, α2, and α3 of a class I MHC molecule of SEQ ID NO: 72,
   exactly one antibody Fc-region, which comprises two disulfide-linked Fc-region polypeptides, whereof the first disulfide-linked Fc-region polypeptide has the amino acid sequence of SEQ ID NO: 97 and the second disulfide-linked Fc-region polypeptide has the amino acid sequence of SEQ ID NO: 98, and
   at least one antigen binding site, which comprises an antibody light chain variable domain comprising an amino acid sequence from the group of SEQ ID NO: 104 to 106 and an antibody heavy chain variable domain comprising an amino acid sequence from the group of SEQ ID NO: 108 to 110, which specifically binds to melanoma-associated chondroitin sulfate proteoglycan (MCSP).
41. A multi-function protein, characterized in that it comprises
   exactly one antigen presenting domain, which comprises in N- to C-terminal direction
      (i) a T-cell response eliciting peptide of SEQ ID NO: 01,
      (ii) a β2-microglobulin of SEQ ID NO: 71, and
      (iii) the extracellular domains α1, α2, and α3 of a class I MHC molecule of SEQ ID NO: 72,
   exactly one antibody Fc-region, which comprises two disulfide-linked Fc-region polypeptides, whereof the first disulfide-linked Fc-region polypeptide has the amino acid sequence of SEQ ID NO: 102 and the second disulfide-linked Fc-region polypeptide has the amino acid sequence of SEQ ID NO: 103, and
   at least one antigen binding site, which specifically binds to melanoma-associated chondroitin sulfate proteoglycan (MCSP), and which comprises an antibody light chain with a variable domain comprising an amino acid sequence from the group of SEQ ID NO: 104 to 106 and an antibody heavy chain with a variable domain comprising an amino acid sequence from the group of SEQ ID NO: 108 to 110.
42. A multi-function protein, characterized in that it comprises
   exactly one antigen presenting domain, which comprises in N- to C-terminal direction
      (i) a T-cell response eliciting peptide of SEQ ID NO: 01,
      (ii) a β2-microglobulin of SEQ ID NO: 71, and
      (iii) the extracellular domains α1, α2, and α3 of a class I MHC molecule of SEQ ID NO: 72,
   exactly one antibody Fc-region, which comprises two disulfide-linked Fc-region polypeptides, whereof the first disulfide-linked Fc-region polypeptide has the amino acid sequence of SEQ ID NO: 97 and the second disulfide-linked Fc-region polypeptide has the amino acid sequence of SEQ ID NO: 98, and
   two antigen binding sites, which specifically bind to melanoma-associated chondroitin sulfate proteoglycan (MCSP), and which each comprise an antibody light chain with a variable domain comprising an amino acid sequence from the group of SEQ ID NO: 105 to 107 and an antibody heavy chain with a variable domain comprising an amino acid sequence from the group of SEQ ID NO: 110 to 112.
43. A multi-function protein, characterized in that it comprises
   exactly one antigen presenting domain, which comprises in N- to C-terminal direction
      (i) a T-cell response eliciting peptide of SEQ ID NO: 01,
      (ii) a β2-microglobulin of SEQ ID NO: 71, and
      (iii) the extracellular domains α1, α2, and α3 of a class I MHC molecule of SEQ ID NO: 72,
   exactly one antibody Fc-region, which comprises two disulfide-linked Fc-region polypeptides, whereof the first disulfide-linked Fc-region polypeptide has the amino acid sequence of SEQ ID NO: 102 and the second disulfide-linked Fc-region polypeptide has the amino acid sequence of SEQ ID NO: 103, and
   at least one antigen binding site, which specifically binds to melanoma-associated chondroitin sulfate proteoglycan (MCSP), and which comprises an antibody light chain with a variable domain comprising an amino acid sequence from the group of SEQ ID NO: 105 to 107 and an antibody heavy chain with a variable domain comprising an amino acid sequence from the group of SEQ ID NO: 110 to 112, whereby the Fc-region of the antibody heavy chain is one of the disulfide-linked Fc-region polypeptides.
44. A multi-function protein, characterized in that it comprises
   exactly one antigen presenting domain, which comprises in N- to C-terminal direction
      (i) a T-cell response eliciting peptide of SEQ ID NO: 01,
      (ii) a β2-microglobulin of SEQ ID NO: 71, and
      (iii) the extracellular domains α1, α2, and α3 of a class I MHC molecule of SEQ ID NO: 72,
   exactly one antibody Fc-region, which comprises two disulfide-linked Fc-region polypeptides, whereof the first disulfide-linked Fc-region polypeptide has the amino acid sequence of SEQ ID NO: 97 and the second disulfide-linked Fc-region polypeptide has the amino acid sequence of SEQ ID NO: 98, and
   at least one antigen binding site, which specifically binds to melanoma-associated chondroitin sulfate proteoglycan (MCSP), and which comprises an antibody light chain with a variable domain comprising an amino acid sequence from the group of SEQ ID NO: 104 to 106 and an antibody heavy chain with a variable domain comprising an amino acid sequence from the group of SEQ ID NO: 108 to 110, whereby the Fc-region of the antibody heavy chain is one of the disulfide-linked Fc-region polypeptides,
   wherein the antigen presenting domain is linked to the N-terminus of one of the variable domains.
45. A multi-function protein, characterized in that it comprises
   exactly one antigen presenting domain, which comprises in N- to C-terminal direction
      (i) a T-cell response eliciting peptide of SEQ ID NO: 01,
      (ii) a β2-microglobulin of SEQ ID NO: 71, and
      (iii) the extracellular domains α1, α2, and α3 of a class I MHC
   exactly one antibody Fc-region, which comprises two disulfide-linked Fc-region polypeptides, whereof the first disulfide-linked Fc-region polypeptide has the amino acid sequence of SEQ ID NO: 102 and the second disulfide-linked Fc-region polypeptide has the amino acid sequence of SEQ ID NO: 103, and
   two antigen binding site, which specifically bind to melanoma-associated chondroitin sulfate proteoglycan (MCSP), and which each comprise an antibody light chain with a variable domain comprising an amino acid sequence from the group of SEQ ID NO: 104 to 106 and an antibody heavy chain with a variable domain comprising an amino acid sequence from the group of SEQ ID NO: 108 to 110, whereby the Fc-regions of the antibody heavy chains are the disulfide-linked Fc-region polypeptides,
   wherein the antigen presenting domain is linked to the N-terminus of one of the variable domains.
46. A multi-function protein, characterized in that it comprises
   exactly one antigen presenting domain, which comprises in N- to C-terminal direction
      (i) a T-cell response eliciting peptide of SEQ ID NO: 01,
      (ii) a β2-microglobulin of SEQ ID NO: 71, and
      (iii) the extracellular domains α1, α2, and α3 of a class I MHC molecule of SEQ ID NO: 72,
   exactly one antibody Fc-region, which comprises two disulfide-linked Fc-region polypeptides, whereof the first disulfide-linked Fc-region polypeptide has the amino acid sequence of SEQ ID NO: 97 and the second disulfide-linked Fc-region polypeptide has the amino acid sequence of SEQ ID NO: 98, and
   two antigen binding site, which specifically bind to melanoma-associated chondroitin sulfate proteoglycan (MCSP), and which each comprise an antibody light chain with a variable domain comprising an amino acid sequence from the group of SEQ ID NO: 105 to 107 and an antibody heavy chain with a variable domain comprising an amino acid sequence from the group of SEQ ID NO: 110 to 112, whereby the Fc-regions of the antibody heavy chains are the disulfide-linked Fc-region polypeptides,
   wherein the antigen presenting domain is linked to the N-terminus of one of the heavy chain variable domains.
47. A multi-function protein, characterized in that it comprises
   exactly one antigen presenting domain, which comprises in N- to C-terminal direction
      (i) a T-cell response eliciting peptide of SEQ ID NO: 01,
      (ii) a β2-microglobulin of SEQ ID NO: 71, and
      (iii) the extracellular domains α1, α2, and α3 of a class I MHC molecule of SEQ ID NO: 72,
   exactly one antibody Fc-region, which comprises two disulfide-linked Fc-region polypeptides, whereof the first disulfide-linked Fc-region polypeptide has the amino acid sequence of SEQ ID NO: 102 and the second disulfide-linked Fc-region polypeptide has the amino acid sequence of SEQ ID NO: 103, and
   two antigen binding site, which specifically bind to melanoma-associated chondroitin sulfate proteoglycan (MCSP), and which each comprise an antibody light chain with a variable domain comprising an amino acid sequence from the group of SEQ ID NO: 105 to 107 and an antibody heavy chain with a variable domain comprising an amino acid sequence from the group of SEQ ID NO: 110 to 112, whereby the Fc-regions of the antibody heavy chains are the disulfide-linked Fc-region polypeptides,
   wherein the antigen presenting domain is linked to the N-terminus of one of the heavy chain variable domains.
48. A multi-function protein, characterized in that it comprises
   one polypeptide chain of SEQ ID NO: 117 or 137,
   one polypeptide chain of SEQ ID NO: 118,
   two polypeptide chains each of SEQ ID NO: 119.
49. The multi-function protein according to any one of items 1 to 48, characterized in that the MCSP binding site comprises an antibody heavy chain variable domain comprising an HVR-H1 comprising the amino acid sequence of SEQ ID NO: 108, an HVR-H2 comprising the amino acid sequence of SEQ ID NO: 109, and an HVR-H3 comprising the amino acid sequence of SEQ ID NO: 110.
50. The multi-function protein according to any one of items 1 to 48, characterized in that the MCSP binding site comprises an antibody light chain variable domain comprising an HVR-L1 comprising the amino acid sequence of SEQ ID NO: 104; an HVR-L2 comprising the amino acid sequence of SEQ ID NO: 105; and an HVR-L3 comprising the amino acid sequence of SEQ ID NO: 106.
51. The multi-function protein according to any one of items 1 to 48, characterized in that the MCSP binding site comprises an antibody heavy chain variable domain comprising an HVR-H1 comprising the amino acid sequence of SEQ ID NO: 108; an HVR-H2 comprising the amino acid sequence of SEQ ID NO: 109; an HVR-H3 comprising the amino acid sequence of SEQ ID NO: 110; and an antibody light chain variable domain comprising an HVR-L1 comprising the amino acid sequence of SEQ ID NO: 104; an HVR-L2 comprising the amino acid sequence of SEQ ID NO: 105; and an HVR-L3 comprising the amino acid sequence of SEQ ID NO: 106.
52. The multi-function protein according to any one of items 1 to 48, characterized in that the MCSP binding site comprises an antibody heavy chain variable domain comprising an HVR-H1 comprising the amino acid sequence of SEQ ID NO: 111, an HVR-H2 comprising the amino acid sequence of SEQ ID NO: 112, and an HVR-H3 comprising the amino acid sequence of SEQ ID NO: 110.
53. The multi-function protein according to any one of items 1 to 48, characterized in that the MCSP binding site comprises an antibody light chain variable domain comprising an HVR-L1 comprising the amino acid sequence of SEQ ID NO: 107; an HVR-L2 comprising the amino acid sequence of SEQ ID NO: 105; and an HVR-L3 comprising the amino acid sequence of SEQ ID NO: 106.
54. The multi-function protein according to any one of items 1 to 48, characterized in that the MCSP binding site comprises an antibody heavy chain variable domain comprising an HVR-H1 comprising the amino acid sequence of SEQ ID NO: 111; an HVR-H2 comprising the amino acid sequence of SEQ ID NO: 112; an HVR-H3 comprising the amino acid sequence of SEQ ID NO: 110; and an antibody light chain variable domain comprising an HVR-L1 comprising the amino acid sequence of SEQ ID NO: 107; an HVR-L2 comprising the amino acid sequence of SEQ ID NO: 105; and an HVR-L3 comprising the amino acid sequence of SEQ ID NO: 106.
55. The multi-function protein according to any one of items 1 to 48, characterized in that the MCSP binding site comprises an antibody heavy chain variable domain having at least 95% sequence identity to the amino acid sequence of SEQ ID NO: 114; and an antibody light chain variable domain having at least 95% sequence identity to the amino acid sequence of SEQ ID NO: 113.
56. The multi-function protein according to any one of items 1 to 48, characterized in that the MCSP binding site comprises an antibody heavy chain variable domain of SEQ ID NO: 114; and an antibody light chain variable domain of SEQ ID NO: 113.
57. The multi-function protein according to any one of items 1 to 48, characterized in that the MCSP binding site comprises SEQ ID NO: 114 and SEQ ID NO: 113.
58. The multi-function protein according to any one of items 1 to 48, characterized in that the MCSP binding site comprises an antibody heavy chain variable domain having at least 95% sequence identity to the amino acid sequence of SEQ ID NO: 116; and an antibody light chain variable domain having at least 95% sequence identity to the amino acid sequence of SEQ ID NO: 115.
59. The multi-function protein according to any one of items 1 to 48, characterized in that the MCSP binding site comprises an antibody heavy chain variable domain of SEQ ID NO: 116; and an antibody light chain variable domain of SEQ ID NO: 115.
60. The multi-function protein according to any one of items 1 to 48, characterized in that the MCSP binding site comprises SEQ ID NO: 116 and SEQ ID NO: 115.
61. A nucleic acid encoding the multi-function protein of any one of items 1 to 60.
62. A host cell comprising the nucleic acid of item 61.
63. A pharmaceutical formulation comprising the multi-function protein according to any one of items 1 to 60 and optionally a pharmaceutically acceptable carrier.
64. The multi-function protein according to any one of items 1 to 60 for use as a medicament.
65. The multi-function protein according to any one of items 1 to 60 for use in treating cancer.
66. The multi-function protein according to any one of items 1 to 60 for use in attracting virus-specific cytotoxic T-cells of an individual to a target.
67. The multi-function protein according to any one of items 1 to 60 for use in removal of cancer cells or virus infected cells.
68. A method for the recombinant production of a multi-function protein according to item 1 comprising the following steps:
   cultivating a eukaryotic cell comprising a nucleic acid according to item 61, and
   recovering the multi-function protein from the cell or the cultivation medium,
   wherein the multi-function protein comprises exactly one antigen presenting domain comprising a β2-microglobulin and the extracellular domains α1, α2 and α3 of a class I MHC molecule.
69. Use of the multi-function protein according to any one of items 1 to 60 in the manufacture of a medicament.
70. The use according to item 69, wherein the medicament is for treatment of cancer.
71. The use according to item 69, wherein the medicament is for attracting virus-specific cytotoxic T-cells of an individual to a target.
72. The use according to item 69, wherein the medicament is for removal cancer cells.
73. A method of attracting virus-specific cytotoxic T-cells of an individual to a target in an individual comprising administering to the individual an effective amount of the multi-function protein according to any one of items 1 to 60 to attract virus-specific cytotoxic T-cells of an individual to a target.
74. A method of removal of cancer cells in an individual comprising administering to the individual an effective amount of the multi-function protein according to any one of items 1 to 60 to remove cancer cells.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### V. EXAMPLES

The following are examples of methods and compositions of the invention. It is understood that various other embodiments may be practiced, given the general description provided above.

### Example 1

### Procedure for Isolation and Stimulation of CMV-Specific CD8 Positive T-Cells from Human Donors

Isolation of PBLs

PBL were isolated by Ficoll gradient centrifugation from human donor blood (Greiner bio-one, Cat. No. 227290). PBLs were cultured in RPMI supplemented with 5% human serum (Sigma Cat. No. H2520), 2 mM L-glutamine (PAN Biotech, Cat. No. P04-80100), 100 μg/ml Penicillin/Streptomycin (Roche, Cat. No. 14001100).

Stimulation of PBLs

Cells (2×10⁷ cells/ml) were cultured in cell culture medium supplemented with 50 μg/ml CMV pp65-derived peptide (SEQ ID NO: 01) for two hours under cell culture conditions (37° C., 5% CO₂, 80% humidity). Thereafter the cell suspension was 20-fold diluted with culture medium and further cultured in flat-bottom 96-well plates at a seeding density of 2×10⁵ cells per 96 well. After 4 to 5 days, 20 U/ml IL-2 (Roche, Cat. No. 11011456001), 25 ng/ml IL-7 (Peprotech, Cat. No. 200-01) and 25 ng/ml IL-15 (Peprotech, Cat. No. 200-15) were added and the cells were cultured for another 7 to 8 days. Stimulation of T-cells is visible under the microscope as cell clusters.

Re-Stimulation of PBLs

T-cells were co-cultured with stimulator cells, which are peptide-pulsed autologous primary PBLs of the same donor (either freshly prepared or derived from frozen stocks). The stimulator cells were pulsed with the peptide as described above. After the two hours of peptide incubation the PBLs were irradiated (4000 rad; STS GmbH OB29 Nr.9510-5) and washed twice in culture medium without peptide. The re-stimulation was carried out in 96 well plates round bottom plates. 8×10⁴ to 1×10⁵ stimulator cells were used per 96 well. Cells from the primary culture were washed twice with culture medium, resuspended in 200 μl culture medium and 80 μl were transferred to each well of the stimulator cells. After 3 days 20 U/ml IL-2, 25 ng/ml IL-7 and 25 ng/ml IL-15 were added. Cells did proliferate and were expanded every 2 to 3 days in new wells with fresh medium.

Analysis of T-Cells

Cells were stained for CD8 expression (BD, Cat. No. 345772) and CMV-specific T-cell receptors (ProImmune, Cat. No. F008-4A-E) and analyzed in FACS.

Cell Culture Medium

RPMI1640 (PAN Biotech, Cat. No. P04-17500), 5% Human Serum (HS; Sigma Cat. No. H2520), 2 mM L-glutamine (PAN Biotech, Cat. No. P04-80100), 100 μg/ml Penicillin/Streptomycin (Roche, Cat. No. 14001100).

Results

FACS analysis of four human donor derived peripheral blood lymphocytes (PBLs) was performed. The cells were labeled with a FITC-conjugated anti-CD8 antibody (BD, Cat. No. 345772) combined with APC-conjugated ProS pentamer (ProImmune, Cat. No. F008-4A-E) to stain T-cells which carry a T-cell receptor (TCR) recognizing MHC-class I (HLA-A*0201) loaded with CMV-derived peptide (NLVPMVATV (SEQ ID NO: 01)). For results see FIGS. 4A-B. At day 0 donor 1 and 4 carry low numbers of CMV-specific CD8 T-cells (0.08% and 0.1%, respectively). Donor 2 and 3 carry a higher number of CMV-specific CD8 T cells in their peripheral blood (0.25% and 3.12%, respectively). Fourteen days later after stimulation with CMV-derived peptide pulsed autologous cells only donors 2 and 3 show a significant increase in CMV-specific CD8 T cells (6.2% and 71.2%, respectively) whereas donors 1 and 4 do not show increased numbers of CMV-specific CD8 T cells (0.01% and 0.05%, respectively). Another 14 days later after a second stimulation with CMV-derived peptide pulsed autologous cells donors 2 and 3 show a further increase in CMV-specific CD8 T cells (15.1% and 96.6%, respectively).

### Example 2

### Cytotoxicity Assay

Acute lymphoblastic leukemia cells MN60 carry the A*0201 HLA-A allele. MN60 cells (1×10⁶ cells/ml) were incubated with 50 μg/ml CMV pp65 peptide (SEQ ID NO: 01) for 45 minutes under cell culture conditions (37° C., 5% CO₂, 80% humidity). The incubation results in a peptide exchange in the HLA-A*0201 peptide binding groove. The peptide exchanged MN60 cells were centrifuged and diluted to a density of 1×10⁶ cells/ml with PBS (PanBiotech, Cat. No. P04-36500) and stained with 1 μM of the cell tracer carboxyfluorescein succinimidyl ester (CFSE, Invitrogen, Cat. No. 34554) 15 minutes at room temperature (RT). Cells were washed thereafter once with PBS and diluted to 1×10⁵ cells/ml with AIM-V media (Gibco, Cat. No. 0870112DK). For the assay MN60 cells (target cells) were co-cultured in 96-well round bottom plates with CMV-specific human donor 3 derived CD8⁺ T-cells (effector cells, see example 1) for four hours under cell culture conditions. The effector to target cell ratio of was 4:1. Dead cells are stained with 1 μl/100 μl propidium iodide (PI, Sigma, Cat. No. P-4864) and were FACS analyzed.

Results

Flow Cytometric Analysis was performed to analyze the cytolytic capability of stimulated CTLs through lysis of MN60 tumor cells loaded with CMV peptide:

A co-culture of MN60 cells not loaded with the CMV-derived peptide was performed. MN60 cells are FITC-positive. Effector cells are FITC-negative. Dead cells are PI positive, alive cells are PI-negative. More than 85% of the MN60 cells are alive when they are not loaded with the CMV-derived peptide (Q2 and Q4).

A co-culture of MN60 cells loaded with the CMV-derived peptide was performed. More than 80% of the MN60 cells are dead (Q2 and Q4) whereas the ratio of alive and dead effector cells is not remarkably altered between the FACS analysis indicating a specific lysis of CMV-peptide-loaded target cells.

Flow Cytometric Analysis to analyze the cytolytic capability of stimulated CTLs through lysis of MN60 tumor cells loaded with CMV peptide depending on the effector to target cell ratio:

The cytotoxic assay was performed as described above. Different effector cell to target cell ratios were applied ranging from 0.5 effector cells per target cell to four effector cells per target cell. Incubation time was four hours. MN60 cells which were not loaded with the CMV-derived peptide do not show an increased number of dead cells with an increased effector to target ratio, i.e. ranging from 8% to 13% with ratio 0.5:1 to 4:1.

Almost 20% of the MN60 cells loaded with CMV-derived peptide are already killed with a low effector to target ratio of 0.5:1 within four hours. The number of dead cells increases steeply with an increase in effector to target ratio reaching up to 83% at a ratio of 4:1 effector cells per target cell.

### Example 3

### DNA Preparation, Transfection, Expression, Purification and Analysis

DNA Preparation

250 ml of overnight bacterial LB culture were harvested and plasmid DNA was extracted according to the manufacturer's protocol (High speed Maxi kit, Qiagen, Cat. No. 12663). The resulting plasmid DNA was eluted in 1 ml TE buffer and DNA concentration was determined by spectrophotometric measurement (Epoch, BioTek).

The final expression vector comprised the following elements:
the endonucleolytic restriction sites HindIII, NheI,
a CMV-promoter,
a 5′UTR 1 (derived from the human CMV),
Intron A,
a 5′UTR 2,
an ampicillin-resistance gene,
a BGH poly A site (bovine growth hormone polyadenylation signal),
pUC Ori.

Amino acid sequences of the elements of the multi-function protein comprising a CMV-derived peptide and IGF1R binding specificity (anti-IGF1R antibody):

CMV pp65 Peptide:
SEQ ID NO: 01
NLVPMVATV
Linker 1:
SEQ ID NO: 82
GGGGSGGGGSGGGGS
β2-microglobulin:
SEQ ID NO: 71
IQRTPKIQVYSRHPAENGKSNFLNCYVSGFHPSDIEVD
LLKNGERIEKVEHSDLSFSKDWSFYLLYYTEFTPTEKD
EYACRVNHVTLSQPKIVKWDRDM
Linker 2:
SEQ ID NO: 83
GGGGSGGGGSGGGGSGGGGS
HLA-A*0201 α1-α3:
SEQ ID NO: 72
GSHSMRYFFTSVSRPGRGEPRFIAVGYVDDTQFVRFDS
DAASQRMEPRAPWIEQEGPEYWDGETRKVKAHSQTH
RVDLGTLRGYYNQSEAGSHTVQRMYGCDVGSDWRF
LRGYHQYAYDGKDYIALKEDLRSWTAADMAAQTTK
HKWEAAHVAEQLRAYLEGTCVEWLRRYLENGKETL
QRTDAPKTHMTHHAVSDHEATLRCWALSFYPAEITLT
WQRDGEDQTQDTELVETRPAGDGTFQKWAAVVVPS
GQEQRYTCHVQHEGLPKPLTLRW
Linker 3:
SEQ ID NO: 73
GS
Linker 4:
SEQ ID NO: 83
GGGGSGGGGSGGGGSGGGGS
Linker 13:
SEQ ID NO: 136
GSG
Ig light chain:
SEQ ID NO: 120
EIVLTQSPATLSLSPGERATLSCRASQSVSSYLAWYQQ
KPGQAPRLLIYDASKRATGIPARFSGSGSGTDFTLTISS
LEPEDFAVYYCQQRSKWPPWTFGQGTKVESKRTVAA
PSVFIFPPSDEQLKSGTASVVCLLNNFYPREAKVQWKV
DNALQSGNSQESVTEQDSKDSTYSLSSTLTLSKADYE
KHKVYACEVTHQGLSSPVTKSFNRGEC
Ig heavy chain (IgG1-L234A, L235A mutant):
SEQ ID NO: 121
QVELVESGGGVVQPGRSQRLSCAASGFTFSSYGMHW
VRQAPGKGLEWVAIIWFDGSSTYYADSVRGRFTISRD
NSKNTLYLQMNSLRAEDTAVYFCARELGRRYFDLWG
RGTLVSVSSASTKGPSVFPLAPSSKSTSGGTAALGCLV
KDYFPEPVTVSWNSGALTSGVHTFPAVLQSSGLYSLSS
VVTVPSSSLGTQTYICNVNHKPSNTKVDKKVEPKSCD
KTHTCPPCPAPEAAGGPSVFLFPPKPKDTLMISRTPEVT
CVVVDVSHEDPEVKFNWYVDGVEVHNAKTKPREEQ
YNSTYRVVSVLTVLHQDWLNGKEYKCKVSNKALPAP
IEKTISKAKGQPREPQVYTLPPSRDELTKNQVSLTCLV
KGFYPSDIAVEWESNGQPENNYKTTPPVLDSDGSFFLY
SKLTVDKSRWQQGNVFSCSVMHEALHNHYTQKSLSL
SPGK
Ig heavy chain (IgG1-L234A, L235A mutant
with knob variation):
SEQ ID NO: 122
QVELVESGGGVVQPGRSQRLSCAASGFTFSSYGMHW
VRQAPGKGLEWVAIIWFDGSSTYYADSVRGRFTISRD
NSKNTLYLQMNSLRAEDTAVYFCARELGRRYFDLWG
RGTLVSVSSASTKGPSVFPLAPSSKSTSGGTAALGCLV
KDYFPEPVTVSWNSGALTSGVHTFPAVLQSSGLYSLSS
VVTVPSSSLGTQTYICNVNHKPSNTKVDKKVEPKSCD
KTHTCPPCPAPEAAGGPSVFLFPPKPKDTLMISRTPEVT
CVVVDVSHEDPEVKFNWYVDGVEVHNAKTKPREEQ
YNSTYRVVSVLTVLHQDWLNGKEYKCKVSNKALPAP
IEKTISKAKGQPREPQVYTLPPCRDELTKNQVSLWCLV
KGFYPSDIAVEWESNGQPENNYKTTPPVLDSDGSFFLY
SKLTVDKSRWQQGNVFSCSVMHEALHNHYTQKSLSL
SPGK
Ig heavy chain (IgG1-L234A, L235A mutant
with hole variation):
SEQ ID NO: 123
QVELVESGGGVVQPGRSQRLSCAASGFTFSSYGMHW
VRQAPGKGLEWVAIIWFDGSSTYYADSVRGRFTISRD
NSKNTLYLQMNSLRAEDTAVYFCARELGRRYFDLWG
RGTLVSVSSASTKGPSVFPLAPSSKSTSGGTAALGCLV
KDYFPEPVTVSWNSGALTSGVHTFPAVLQSSGLYSLSS
VVTVPSSSLGTQTYICNVNHKPSNTKVDKKVEPKSCD
KTHTCPPCPAPEAAGGPSVFLFPPKPKDTLMISRTPEVT
CVVVDVSHEDPEVKFNWYVDGVEVHNAKTKPREEQ
YNSTYRVVSVLTVLHQDWLNGKEYKCKVSNKALPAP
IEKTISKAKGQPREPQVCTLPPSRDELTKNQVSLSCAV
KGFYPSDIAVEWESNGQPENNYKTTPPVLDSDGSFFLV
SKLTVDKSRWQQGNVFSCSVMHEALHNHYTQKSLSL
SPGK
Ig heavy chain Fc-region (IgG1-L234A,
L235A mutant Fc-region knob variant):
SEQ ID NO: 124
EPKSADKTHTCPPCPAPEAAGGPSVFLFPPKPKDTLMI
SRTPEVTCVVVDVSHEDPEVKFNWYVDGVEVHNAKT
KPREEQYNSTYRVVSVLTVLHQDWLNGKEYKCKVSN
KALPAPIEKTISKAKGQPREPQVYTLPPCRDELTKNQV
SLWCLVKGFYPSDIAVEWESNGQPENNYKTTPPVLDS
DGSFFLYSKLTVDKSRWQQGNVFSCSVMHEALHNHY
TQKSLSLSPGK
scFv:
SEQ ID NO: 125
QVELVESGGGVVQPGRSQRLSCAASGFTFSSYGMHW
VRQAPGKCLEWVAIIWFDGSSTYYADSVRGRFTISRD
NSKNTLYLQMNSLRAEDTAVYFCARELGRRYFDLWG
RGTLVSVSSGGGGSGGGGSGGGGSGGGGSEIVLTQSP
ATLSLSPGERATLSCRASQSVSSYLAWYQQKPGQAPR
LLIYDASKRATGIPARFSGSGSGTDFTLTISSLEPEDFAV
YYCQQRSKWPPWTFGCGTKVESK

Amino acid sequences of the elements of the multi-function protein comprising a CMV-derived peptide and MCSP binding specificity (anti-MCSP antibody):

CMV pp65 Peptide:
SEQ ID NO: 01
NLVPMVATV
Linker 1:
SEQ ID NO: 82
GGGGSGGGGSGGGGS
β2-microglobulin:
SEQ ID NO: 71
IQRTPKIQVYSRHPAENGKSNFLNCYVSGFHPSDIEVD
LLKNGERIEKVEHSDLSFSKDWSFYLLYYTEFTPTEKD
EYACRVNHVTLSQPKIVKWDRDM
Linker 2:
SEQ ID NO: 83
GGGGSGGGGSGGGGSGGGGS
HLA-A*0201 α1-α3:
SEQ ID NO: 72
GSHSMRYFFTSVSRPGRGEPRFIAVGYVDDTQFVRFDS
DAASQRMEPRAPWIEQEGPEYWDGETRKVKAHSQTH
RVDLGTLRGYYNQSEAGSHTVQRMYGCDVGSDWRF
LRGYHQYAYDGKDYIALKEDLRSWTAADMAAQTTK
HKWEAAHVAEQLRAYLEGTCVEWLRRYLENGKETL
QRTDAPKTHMTHHAVSDHEATLRCWALSFYPAEITLT
WQRDGEDQTQDTELVETRPAGDGTFQKWAAVVVPS
GQEQRYTCHVQHEGLPKPLTLRW
Linker 3:
SEQ ID NO: 73
GS
Linker 4:
SEQ ID NO: 83
GGGGSGGGGSGGGGSGGGGS
Linker 13:
SEQ ID NO: 136
GSG
Ig light chain: MHCI-0008
SEQ ID NO: 126
DIVLTQSPSSLSASLGDRVTISCSASQGIRNYLNWYQQ
RPDGTVKLLIYYTSSLHSGVPSRFSGSGSGTDYSLTISN
LEPEDIATYYCQQYSKLPWTFGGGTKLEIKRTVAAPSV
FIFPPSDEQLKSGTASVVCLLNNFYPREAKVQWKVDN
ALQSGNSQESVTEQDSKDSTYSLSSTLTLSKADYEKH
KVYACEVTHQGLSSPVTKSFNRGEC
Ig light chain: MHCI-0030 and MHCI-0031
SEQ ID NO: 127
DIQMTQSPSSLSASVGDRVTITCRASQGIRNYLNWYQ
QKPGKAPKLLIYYTSSLHSGVPSRFSGSGSGTDFTLTIS
SLQPEDFATYYCQQYSKLPWTFGQGTKVEIKRTVAAP
SVFIFPPSDEQLKSGTASVVCLLNNFYPREAKVQWKV
DNALQSGNSQESVTEQDSKDSTYSLSSTLTLSKADYE
KHKVYACEVTHQGLSSPVTKSFNRGEC
Ig heavy chain (IgG1-L234A, L235A mutant):
MHCI-0008
SEQ ID NO: 128
EVQLQESGPGLVKPSQSLSLTCSVTGYSITSGYYWNWI
RQFPGNKLEWMGYITYDGSNNYNPSLKNRISITRDTSK
NQFFLKLNSVTTEDTATYYCADFDYWGQGTTLTVSSA
STKGPSVFPLAPSSKSTSGGTAALGCLVKDYFPEPVTV
SWNSGALTSGVHTFPAVLQSSGLYSLSSVVTVPSSSLG
TQTYICNVNHKPSNTKVDKKVEPKSCDKTHTCPPCPA
PEAAGGPSVFLFPPKPKDTLMISRTPEVTCVVVDVSHE
DPEVKFNWYVDGVEVHNAKTKPREEQYNSTYRVVSV
LTVLHQDWLNGKEYKCKVSNKALPAPIEKTISKAKGQ
PREPQVYTLPPSRDELTKNQVSLTCLVKGFYPSDIAVE
WESNGQPENNYKTTPPVLDSDGSFFLYSKLTVDKSRW
QQGNVFSCSVMHEALHNHYTQKSLSLSPGK
Ig heavy chain (IgG1-L234A, L235A mutant):
MHCI-0030 and MHCI-0031
SEQ ID NO: 129
QVQLQESGPGLVKPSQTLSLTCTVSGGSITSGYYWNW
IRQHPGKGLEWIGYITYDGSNNYNPSLKSRVTISRDTS
KNQFSLKLSSVTAADTAVYYCADFDYWGQGTLVTVS
SASTKGPSVFPLAPSSKSTSGGTAALGCLVKDYFPEPV
TVSWNSGALTSGVHTFPAVLQSSGLYSLSSVVTVPSSS
LGTQTYICNVNHKPSNTKVDKKVEPKSCDKTHTCPPC
PAPEAAGGPSVFLFPPKPKDTLMISRTPEVTCVVVDVS
HEDPEVKFNWYVDGVEVHNAKTKPREEQYNSTYRVV
SVLTVLHQDWLNGKEYKCKVSNKALPAPIEKTISKAK
GQPREPQVYTLPPSRDELTKNQVSLTCLVKGFYPSDIA
VEWESNGQPENNYKTTPPVLDSDGSFFLYSKLTVDKS
RWQQGNVFSCSVMHEALHNHYTQKSLSLSPGK
Ig heavy chain (IgG1-L234A, L235A mutant
with knob variation):
MHCI-0008
SEQ ID NO: 130
EVQLQESGPGLVKPSQSLSLTCSVTGYSITSGYYWNWI
RQFPGNKLEWMGYITYDGSNNYNPSLKNRISITRDTSK
NQFFLKLNSVTTEDTATYYCADFDYWGQGTTLTVSSA
STKGPSVFPLAPSSKSTSGGTAALGCLVKDYFPEPVTV
SWNSGALTSGVHTFPAVLQSSGLYSLSSVVTVPSSSLG
TQTYICNVNHKPSNTKVDKKVEPKSCDKTHTCPPCPA
PEAAGGPSVFLFPPKPKDTLMISRTPEVTCVVVDVSHE
DPEVKFNWYVDGVEVHNAKTKPREEQYNSTYRVVSV
LTVLHQDWLNGKEYKCKVSNKALPAPIEKTISKAKGQ
PREPQVYTLPPCRDELTKNQVSLWCLVKGFYPSDIAV
EWESNGQPENNYKTTPPVLDSDGSFFLYSKLTVDKSR
WQQGNVFSCSVMHEALHNHYTQKSLSLSPGK
Ig heavy chain (IgG1-L234A, L235A mutant
with knob variation):
MHCI-0030 and MHCI-0031
SEQ ID NO: 131
QVQLQESGPGLVKPSQTLSLTCTVSGGSITSGYYWNW
IRQHPGKGLEWIGYITYDGSNNYNPSLKSRVTISRDTS
KNQFSLKLSSVTAADTAVYYCADFDYWGQGTLVTVS
SASTKGPSVFPLAPSSKSTSGGTAALGCLVKDYFPEPV
TVSWNSGALTSGVHTFPAVLQSSGLYSLSSVVTVPSSS
LGTQTYICNVNHKPSNTKVDKKVEPKSCDKTHTCPPC
PAPEAAGGPSVFLFPPKPKDTLMISRTPEVTCVVVDVS
HEDPEVKFNWYVDGVEVHNAKTKPREEQYNSTYRVV
SVLTVLHQDWLNGKEYKCKVSNKALPAPIEKTISKAK
GQPREPQVYTLPPCRDELTKNQVSLWCLVKGFYPSDI
AVEWESNGQPENNYKTTPPVLDSDGSFFLYSKLTVDK
SRWQQGNVFSCSVMHEALHNHYTQKSLSLSPGK
Ig heavy chain (IgG1-L234A, L235A mutant
with hole variation):
MHCI-0008
SEQ ID NO: 132
EVQLQESGPGLVKPSQSLSLTCSVTGYSITSGYYWNWI
RQFPGNKLEWMGYITYDGSNNYNPSLKNRISITRDTSK
NQFFLKLNSVTTEDTATYYCADFDYWGQGTTLTVSSA
STKGPSVFPLAPSSKSTSGGTAALGCLVKDYFPEPVTV
SWNSGALTSGVHTFPAVLQSSGLYSLSSVVTVPSSSLG
TQTYICNVNHKPSNTKVDKKVEPKSCDKTHTCPPCPA
PEAAGGPSVFLFPPKPKDTLMISRTPEVTCVVVDVSHE
DPEVKFNWYVDGVEVHNAKTKPREEQYNSTYRVVSV
LTVLHQDWLNGKEYKCKVSNKALPAPIEKTISKAKGQ
PREPQVCTLPPSRDELTKNQVSLSCAVKGFYPSDIAVE
WESNGQPENNYKTTPPVLDSDGSFFLVSKLTVDKSRW
QQGNVFSCSVMHEALHNHYTQKSLSLSPGK
Ig heavy chain (IgG1-L234A, L235A mutant
with hole variation):
MHCI-0030 and MHCI-0031
SEQ ID NO: 133
QVQLQESGPGLVKPSQTLSLTCTVSGGSITSGYYWNW
IRQHPGKGLEWIGYITYDGSNNYNPSLKSRVTISRDTS
KNQFSLKLSSVTAADTAVYYCADFDYWGQGTLVTVS
SASTKGPSVFPLAPSSKSTSGGTAALGCLVKDYFPEPV
TVSWNSGALTSGVHTFPAVLQSSGLYSLSSVVTVPSSS
LGTQTYICNVNHKPSNTKVDKKVEPKSCDKTHTCPPC
PAPEAAGGPSVFLFPPKPKDTLMISRTPEVTCVVVDVS
HEDPEVKFNWYVDGVEVHNAKTKPREEQYNSTYRVV
SVLTVLHQDWLNGKEYKCKVSNKALPAPIEKTISKAK
GQPREPQVCTLPPSRDELTKNQVSLSCAVKGFYPSDIA
VEWESNGQPENNYKTTPPVLDSDGSFFLVSKLTVDKS
RWQQGNVFSCSVMHEALHNHYTQKSLSLSPGK
Ig heavy chain Fc-region (IgG1-L234A,
L235A mutant Fc-region knob variant):
SEQ ID NO: 124
EPKSADKTHTCPPCPAPEAAGGPSVFLFPPKPKDTLMI
SRTPEVTCVVVDVSHEDPEVKFNWYVDGVEVHNAKT
KPREEQYNSTYRVVSVLTVLHQDWLNGKEYKCKVSN
KALPAPIEKTISKAKGQPREPQVYTLPPCRDELTKNQV
SLWCLVKGFYPSDIAVEWESNGQPENNYKTTPPVLDS
DGSFFLYSKLTVDKSRWQQGNVFSCSVMHEALHNHY
TQKSLSLSPGK
scFv: MHCI-0008
SEQ ID NO: 134
EVQLQESGPGLVKPSQSLSLTCSVTGYSITSGYYWNWI
RQFPGNKLEWMGYITYDGSNNYNPSLKNRISITRDTSK
NQFFLKLNSVTTEDTATYYCADFDYWGQGTTLTVSSG
GGGSGGGGSGGGGSGGGGSDIVLTQSPSSLSASLGDR
VTISCSASQGIRNYLNWYQQRPDGTVKLLIYYTSSLHS
GVPSRFSGSGSGTDYSLTISNLEPEDIATYYCQQYSKLP
WTFGGGTKLEIK
scFv: MHCI-0030 and MHCI-0031
SEQ ID NO: 135
QVQLQESGPGLVKPSQTLSLTCTVSGGSITSGYYWNW
IRQHPGKGLEWIGYITYDGSNNYNPSLKSRVTISRDTS
KNQFSLKLSSVTAADTAVYYCADFDYWGQGTLVTVS
SGGGGSGGGGSGGGGSGGGGSDIQMTQSPSSLSASVG
DRVTITCRASQGIRNYLNWYQQKPGKAPKLLIYYTSSL
HSGVPSRFSGSGSGTDFTLTISSLQPEDFATYYCQQYS
KLPWTFGQGTKVEIK

Transfection

HEK 293 cells were diluted to 8×10⁵ cells/ml the day before transfection. About 1 to 1.6×10⁶ cells/ml were transfected according to the manufacturer's protocol. For a final transfection volume of 30 ml, 30 μg DNA were diluted to a final volume of 1 ml with Opti-MEM® I Reduced Serum Medium (Gibco, Cat. No. 31985070). 2 μl of 293Fectin™ Reagent (Invitrogen, Cat. No. 12347019) per 1 μg DNA were equally diluted to a final volume of 1 ml with Opti-MEM® medium and incubated for 5 minutes. After incubation the diluted DNA was added to the diluted 293Fectin™Reagent, gently mixed, incubated for another 20-30 minutes and afterwards drop wise pipetted to 28 ml of the HEK 293 cells to obtain a final volume of 30 ml. The cells were incubated under cell culture condition (37° C., 8% CO₂, 80% humidity) on an orbital shaker rotating at 125 rpm and harvested after 72 hours. The harvest was centrifuged for 10 minutes at 1000 rpm, for 10 minutes at 3000 rpm and filtered through a 22 μm sterile filter (Millipore, Cat. No. SCGPU05RE).

Western Blotting

500 μl of cell culture supernatant was concentrated with Pall Nanosep Omega-Membrane 30 KD Centrifugal Devices (Pall, Cat. No. OD030C33) to a volume of 50 μl. 17.5 μl of each concentrate was diluted to a final volume of 25 μl with 4×XT Sample Buffer (Bio Rad, Cat. No. 161-0791) and 20×XT Reducing Agent (BioRad, Cat. No. 161-0792), heated for 8 minutes at 96° C. and applied on a 4-12% Criterion XT Precast Gel (Cat. No. 345-0124). Blotting was performed with Trans-Blot SD semi-dry Transfer Cell (BioRad) at 20 V for 30 minutes on a Trans-blot Pure Nitrocellulose membrane (0.45 μm) (BioRad, Cat. No. 162-0117). Blocking of the membrane was performed with 1× Western Blocking Reagent (Roche, Cat. No. 11921681001) for one hour at room temperature. Staining was performed with peroxidase conjugated polyclonal rabbit anti-human κ-light chain (DAKO, Cat. No. P0129, diluted 1:3000) and polyclonal rabbit anti-human IgG antibody HRP conjugate (DAKO, Cat. No. P0214, diluted 1:5000) for one hour at room temperature. Luminescence detection was carried out with LUMI-Imager F1 (Roche).

Purification

Cells were removed from culture medium by centrifugation. Multi-function proteins were purified from supernatants by protein A affinity chromatography (MabSelect-Sepharose on an AKTA-Avant). Eluted multi-function proteins were concentrated with Amicon centrifugation tubes to a protein concentration of 3 mg/ml. An aliquot was analyzed on a size exclusion chromatography (HPLC TSKgel GFC300 Sys89). Preparative SEC on a Superdex 200 was performed to remove aggregates and buffer the fusion proteins in 20 mM histidine, 140 mM NaCl, pH 6.0. Eluted multi-function proteins were concentrated with Amicon centrifugation tube to a protein concentration of 1 mg/ml and sterile filtered (0.2 μm pore size).

Analytics

Multi-function protein samples were analyzed by OD280 using a UV spectrophotometer to determine the protein concentration in solution. The extinction coefficient required for this was calculated from the amino acid sequence according to Pace, C. N., et al., Protein Science 4 (1995) 2411-2423). Size-exclusion chromatography (SE-HPLC) was performed on TSK-Gel1300SWXL or Superdex 200 columns with a 0.2 M potassium phosphate buffer, comprising 0.25 M KCl, pH 7.0 as mobile phase in order to determine the content of monomeric, aggregated and degraded species in the samples. Sodium dodecyl sulfate (SDS) polyacrylamide gel electrophoresis (reducing and non-reducing) was performed to analyze the purity of the multi-function protein preparations with regard to product-related degradation products and unrelated impurities. Electrospray ionization mass spectrometry (ESI-MS) was performed with reduced (TCEP) and deglycosylated (N-glycosidase F) samples to confirm the correct mass/identity of each chain and detect chemical modifications. ESI-MS of the deglycosylated samples was carried out to analyze the nature and quality of the fully assembled protein and detect potential product-related side products.

Method SDS-PAGE and Coomassie Staining
Device: Invitrogen XCell Sure Lock Mini-Cell
Gel: 4-20% Tris-Glycine Gel, Invitrogen EC6025BOX
Buffer: Tris-Glycine SDS Running Buffer (10×), Invitrogen LC2675-5
Sample buffer: Tris-Glycine SDS Sample Buffer (2×), Invitrogen LC2676
Reducing buffer: NuPAGE Sample Reducing Agent (10×), Invitrogen NP0004
Molecular Weight Marker: Mark 12, MW Standard, Invitrogen LC5677

Protein Sample Preparation

The sample was adjusted to a protein concentration of 1 mg/ml with buffer. For sample reduction the following procedure was carried out:
reduction buffer: 4 ml Sample buffer (2×) and 1 ml reducing buffer (10×),
dilute sample 1:1 with reduction buffer,
incubate for 5 minutes at 70° C.

The gel electrophoresis was carried out at 125 V for 90 minutes. The gels were stained with Simply Blue Safe Stain (Invitrogen, Cat. No. LC6065).

Results

TABLE

| | polypeptides comprised in the | | |
|---|---|---|---|
| | multi-function protein with | | |
| No. | IGF-1R binding specificity | scheme | yield |
| 1 | 1. [CMV-pp65-peptide]-[linker 1]-[β2-microglobulin]-[linker 2]-[HLA-A-α1-α2-α3]-[linker 3]-[IgG1-L234A, L235A mutant with hole variation] 2. Ig heavy chain (IgG1- L234A, L235A mutant with knob variation) 3. Ig light chain | 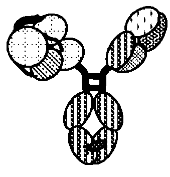 | 5 mg/l |
| 2 | A: 1. [CMV-pp65-peptide]-[linker 1]-[β2-microglobulin]-[linker 2]-[HLA-A-α1-α2-α3]-[linker 3]-[IgG1-L234A, L235A mutant with hole variation] 2. IgG1-L234A, L235A mutant Fc-region knob variant 3. Ig light chain | 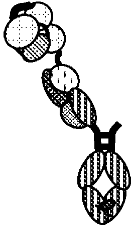 | A: 5-18 mg/l |
| | | A | |
| | B: Fusion to the Ig light chain | 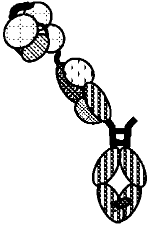 | |
| | | B | |
| 3 | A: 1. [CMV-pp65-peptide]-[linker 1]-[β2-microglobulin]-[linker 2]-[HLA-A-α1-α2-α3]-[linker 3]-[IgG1-L234A, L235A mutant with hole variation] 2. IgG1-L234A, L235A mutant with knob variation 3. Ig light chain | 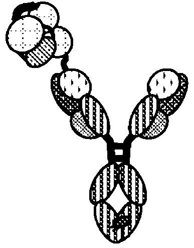 | A: 4-23 mg/l |
| | | A | |
| | B: Fusion to the Ig light chain | 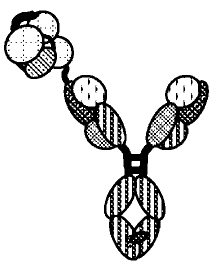 | |
| | | B | |
| 4 | 1. [CMV-pp65-Peptide]-[Linker 1]-[β2-microglobulin]-[Linker 2]-[HLA-A-α1-α2-α3]-[Linker 3]-[IgG1-L234A, L235A mutant with hole variation] 2. IgG1-L234A, L235A mutant with knob variation | 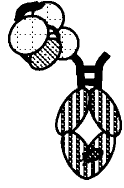 | 4 mg/l |
| 5 | 1. [CMV-pp65-peptide]-[linker 1]-[β2-microglobulin]-[linker 2]-[HLA-A-α1-α2-α3]-[linker 3]-[IgG1-L234A, L235 A-Fc-region] | 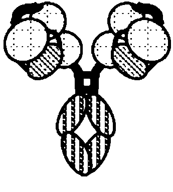 | 4 mg/l |
| 6 | 1. [CMV-pp65-peptide]-[linker 1]-[β2-microglobulin]-[linker 2]-[HLA-A-α1-α2-α3]-[linker 3]-[IgG1-L234A, L235A mutant Fc-region]-[linker 4]-[scFv] | 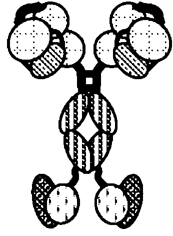 | <1 μg/l |
| 7 | A: 1. [CMV-pp65-peptide]- [linker 1]-[β2-microglobulin]- [linker 2]-[HLA-A-α1-α2-α3]- [linker 3]-[IgG1- L234A, L235A mutant] 2. Ig light chain | 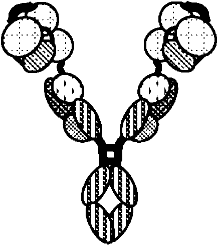 | <1 μg/l |
| | | A | |
| | B: Fusion to the Ig light chain | 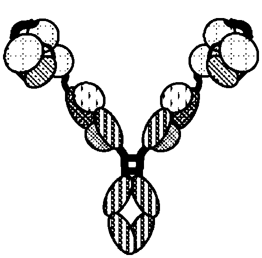 | |
| | | B | |

The SDS gel with Coomassie staining and the corresponding SEC chromatograms of selected multi-function proteins with a structure corresponding to number 1, 2A and 3A according to the previous table are shown in FIGS. 5A, 5B and FIGS. 6A-1, 6A-2, 6B-1 and 6B-2. It can be seen that defined multi-function proteins can be obtained.

### Example 4

### Binding of MHC-I-Anti-IGF-1R Multi-Function Protein to Human IGF-1R Positive Cell Line

H460M2 cells were diluted to 8×10⁵ cells/ml in AIM-V medium (Gibco, Cat. No. 0870112DK). 500 μl of the cell suspension was stained with 10 μg of a MHC-I-anti-IGF-1R multi-function protein as reported herein either at 4° C. or 37° C. for one hour. Thereafter cells were washed once with ice-cold AIM-V medium and stained with a second antibody, which was a goat F(ab′)₂ anti-human IgG (H+L) antibody conjugated to R-PE (Dianova, Cat. No. 109-116-088, dilution 1:50) for 30 minutes at 4° C. Thereafter cells were washed once with ice-cold AIM-V medium and fluorescence was measured via FACS Canto II (BD Bioscience) with gating on living cells. A bispecific antibody served as Isotype control, an anti IGF-1R antibody (see e.g. WO 2004/087756 and WO 2007/115814) served as positive control.

Results

Considering the shift in the PE-fluorescence measurement (X-axis), the MHC-I-anti-IGF-1R multi-function protein shows no visible difference in binding to H460M2 target cells in comparison to the control antibody. There is also no difference whether the incubation with the MHC-I-anti-IGF-1R multi-function protein is accomplished at 4° C. or 37° C. Neither the incubation with the isotype control nor with the fluorescence labeled secondary antibody alone shows any shift in the PE fluorescence measurement. Despite the fusion of the class I MHC molecule the antibody variable domain of the MHC-I-anti-IGF-1R multi-function protein herein still binds to the H460M2 target cells.

### Example 5

### In Vitro Removal of Antigen Expressing Cells

I24 target cells (1×10⁵ cells/ml) were seeded in cell culture media (RPMI 1640 supplemented with 2 mM L-glutamine, 1 mM sodium pyruvate, 0.1 mM NEAA, and 10% (v/w) FCS) on WillCo Glass Bottom Dishes (FA. WillCo Wells BV, REF GWST-3522) for 24 to 48 hours. WillCo Glass Bottom Dishes were pre-coated with 50 μg/ml poly-L-lysine hydrochloride (Sigma Aldrich, Cat # P2658) per dish for 30 min. After coating the dishes were thoroughly rinsed with sterile tissue culture grade water and dried for two hours.

After the cultivation cell culture media was removed and the IGF-1R binding multi-function protein comprising one [CMV-pp65-peptide]-[linker 1]-[β2-microglobulin]-[linker 2]-[HLA-A-α1-α2-α3]-[linker 3]-[IgG1-L234A, L235A mutant with hole variation] fusion polypeptide, one IgG1-L234A, L235A mutant Fc-region knob variant disulfide-linked polypeptide and one Ig light chain, wherein the multi-function protein specifically binds to human IGF-1R as reported herein (see e.g. Example 3) was added in a final concentration of 5 μg/ml in 3 mM K⁺ Krebs Ringer HEPES Buffer pH 7.3 (supplemented with 0.5 mM DL-dithiothreitol, 1 mM ascorbic acid, and 4 mM glutathione).

T-cells were added in a target cell to effector cell ration of 1:10. Imaging was performed for 4 hours with a Zeiss Axiovert 135 microscope.

Results

The IGF-1R binding multi-function protein mediated lysis of human IGF-1R expressing I24 3T3 cells (large adherently growing cells). Lysis is mediated by human CMV-specific T-cells (small cells either round shaped or amoeboid migrating cells). I24 cells are incubated with the multi-function protein at a concentration of 5 μg/ml and human CMV-specific T-cells (pre-activated with HLA-A0201⁺/CMV peptide pulsed APCs). Note the interaction of the I24 cells with the T-cells at 56 min and 76 min and subsequently the collapse of the I24 cell after 125 min.

A control showing the absence of lysis of I24 3T3 cells (large adherently growing cells, white arrowhead) through human CMV-specific T-cells (small cells either round shaped or amoeboid migrating cells) in the absence of an antigen binding multi-function protein as reported herein was performed. I24 cells are incubated with specific cytotoxic T-cells (pre-activated with HLA-A0201⁺/CMV peptide pulsed APCs). Time lapse is indicated below the respective picture.

### Example 6

### Cytotoxicity Assay

Cell culture medium (50 μl) was pipetted into each well of an Xcelligence 96well E-plate (Roche, Cat #05232368001) to perform background measurement.

I24 cells were diluted to 1×10⁶ cells/ml in cell culture media (RPMI 1640, 2 mM L-glutamine, 1 mM Sodium pyruvate, 0.1 mM NEAA, 10% (v/w) FCS) and 50 μl (2×10⁴ cells/well) were pipetted in each well of an Xcelligence 96well plate to a final volume of 100 μl and cultivated for 24 hours (37° C., 8% CO₂, 80% humidity). After 24 hours the medium was removed and the cells were washed with 200 μl AIM-V (Serum Free Medium (Invitrogen) T-cell medium (Cat-No): 12055-083) medium. The IGF-1R binding multi-function protein comprising one [CMV-pp65-peptide]-[linker 1]-[β2-microglobulin]-[linker 2]-[HLA-A-α1-α2-α3]-[linker 3]-[IgG1-L234A, L235A mutant with hole variation] fusion polypeptide, one IgG1-L234A, L235A mutant Fc-region knob variant disulfide-linked polypeptide and one Ig light chain, wherein the multi-function protein specifically binds to human IGF-1R, was added to the washed target cells in a final concentration of 1 μg/ml in AIM-V medium. Effector cells in the respectable ratio were added in AIM-V media to a final volume of 150 μl. Afucosylated IgG1 monoclonal antibody directed against human IGF-1R (anti-IGF-1R antibody-afucosylated) and non-binding human anti-digoxigenin antibody (anti-digoxygenin antibody) served as Isotype control and specific antibody control, respectively. Measurement was performed for 6 to 9 hours respectively with the Xcelligence System (Roche).

Results

The IGF-1R binding multi-function protein triggers lysis of H460M2 tumor cells through human CMV-specific T-cells.

Tumor cells were incubated for 4 hours with 1 μg/ml of the multi-function protein comprising one [CMV-pp65-peptide]-[linker 1]-[β2-microglobulin]-[linker 2]-[HLA-A-α1-α2-α3]-[linker 3]-[IgG1-L234A, L235A mutant with hole variation] fusion polypeptide, one IgG1-L234A, L235A mutant Fc-region knob variant disulfide-linked polypeptide and one Ig light chain, wherein the multi-function protein specifically binds to human IGF-1R, and specific T-cells in the respective ratio (1:1.5 to 1:0.5) (see FIGS. 8A-C). Percentage of lysis is denoted above the respective bars. Afucosylated IgG1 monoclonal antibody directed against human IGF-1R (MAB IGF-1R-afu) did not trigger a significant tumor cell lysis.

The multi-function protein as reported herein triggers lysis of I24 3T3 target cells through human CMV-specific T-cells.

Target cells were incubated for 4 hours with 1 μg/ml of an antigen binding multi-function protein comprising one [CMV-pp65-peptide]-[linker 1]-[β2-microglobulin]-[linker 2]-[HLA-A-α1-α2-α3]-[linker 3]-[IgG1-L234A, L235A mutant with hole variation] fusion polypeptide, one IgG1-L234A, L235A mutant Fc-region knob variant disulfide-linked polypeptide and one Ig light chain, wherein the multi-function protein specifically binds to human IGF-1R, and specific T-cells in the respective ratio (1:1.5 to 1:0.5) (see FIGS. 9A-C). Percentage of lysis is denoted above the respective bars. Afucosylated IgG1 monoclonal antibody directed against human IGF-1R (anti-IGF-1R antibody-afucosylated) and non-binding human anti-Digoxigenin antibody (anti-digoxygenin antibody) did not trigger a significant target cell lysis.

### Example 7

### In Vitro Efficacy

IGF-1R positive lung adenocarcinoma cell line H460M2 was incubated with 1 μg/ml of a multi-function protein comprising an hCMV-derived peptide and an anti-IGF-1R antibody and human CMV-specific CD8-positive T-cells at a low effector cell to target cell ratio (1.5 to 0.5 specific T-cells per tumor cell). Control antibody was a glyco-engineered anti-IGF-1R antibody. The incubation time was 6 hours. The incubation with multi-function protein results in a potent removal of H460M2 tumor cells.

### Example 8

### Binding of Different MHC-I-Anti-MCSP Multi-Function Protein to MCSP Positive Target Cells

Colo38 cells were incubated for 5 min. with Accutase (PAA, Cat.# L11-007) to obtain a single cell suspension. 2×10⁵ cells per vial were incubated with 1 μg/ml MHC-I-anti-MCSP multi-function protein construct in 100 n1 PBS/2% FCS for 45 min. at 4° C. After incubation cells were washed with 1 ml cold PBS/2% FCS and centrifuged for 7 min with 910 rpm. Cells were resuspended in 100 μl PBS/2% FCS with secondary antibody (goat anti-human IgG1 antibody-PE conjugate, Jackson Lab., Cat.#109-116-088) at 2 μg/ml and incubated for another 45 min. at 4° C. Cells were washed twice with 1 ml PBS %2% FCS and measured with BD Canto II Flow Cytometer. The results are shown in FIG. 7.

### Example 9

### Incubation of MCSP Positive Cells with MHC-I-Anti-MCSP Multi-Function Proteins

Colo38 or WM266-4 cells were incubated for 5 min. with Accutase (PAA, Cat.# L11-007) to obtain a single cell suspension. 2×10⁴ cells of the Colo38 cell line or 1×10⁴ cells of the WM266-4 cell line per well were incubated for 24 h in Eplates96 (Roche, Cat.#05232368001) in 100 n1 of the respective cell culture medium (Colo38 cell line: RPMI1640 supplemented with 2 mM glutamine, 10% FCS; WM-266-4 cell line: RPMI1640 supplemented with 2 mM glutamine, 10% FKS, 2 mM sodium pyruvate, 2 mM NEAA) and adherence (impedance) was measured every 15 min with ACEA technology (Xcelligence RTCA). After 24 h (undisturbed growth phase) the cells were washed with 200 μl of AIMV-medium (Gibco, Cat.#0870112DK). MHC-I-anti-MCSP multi-function proteins were added in a final concentration of 1 μg/ml together with stimulated T-cells or PBMCs in different ratios to a final volume of 150 μl in AIMV-medium to the cells. Incubation was continued for another 4 to 48 hours with simultaneous ACEA measurement every 5 minutes. The read-out is based on impedance measurement, detecting lysed or collapsed cells as detached from the Eplate bottom. The cell index has been normalized to 1 at the first measurement point after addition of the multi-function protein. The results for 1 μg/ml multi-function protein concentration (MHCI-0008 (1), MHCI-0010 (2), MHCI-0030 (3), MHCI-0031 (4)), effector to target cell ratio of 10:1; PBMCs from Donor 3 (200.000 cells, Donor 3 is CMV-positive but EBV negative) and melanoma tumor cell line Colo38 (20.000 cells) and per 96 well, data are triplicates is shown in FIG. 14. The results for 1 μg/ml multi-function protein concentration (MHCI-0008 (1), MHCI-0010 (2), PBMCs only (3)), effector to target cell ratio of 10:1; PBMCs from Donor 3 (200.000 cells, Donor 3 is CMV-positive but EBV negative) and melanoma tumor cell line Colo38 (20.000 cells) and per 96 well, data are triplicates is shown in FIG. 15A (Colo38) and 15B (WM266).

### Example 10

### Cytotoxicity Assay

Cell culture medium (50 μl) was pipetted into each well of an Xcelligence 96well E-plate (Roche, Cat #05232368001) to perform background measurement.

Colo38 cells were diluted to 1×10⁶ cells/ml in cell culture media (RPMI1640 supplemented with 2 mM glutamine, 10% FCS) and 50 μl (2×10⁴ cells/well) were pipetted in each well of an Xcelligence 96well plate to a final volume of 100 μl and cultivated for 24 hours (37° C., 8% CO₂, 80% humidity). After 24 hours the medium was removed and the cells were washed with 200 μl AIM-V (Serum Free Medium (Invitrogen) T-cell medium (Cat-No): 12055-083) medium. The MCSP binding multi-function proteins MHCI-0008 (monovalent, CMV peptide loaded), MHCI-0010 (monovalent, EBV peptide loaded control), MHC-0026 (bivalent, CMV peptide loaded, non-binding control), MHCI-0030 (monovalent, CMV peptide loaded, active) and MHCI-0031 (bivalent, CMV peptide loaded, active) were individually added to the washed target cells in a final concentration of 1 μg/ml in AIM-V medium. Effector cells in the respectable ratio of 10:1 (E:T) were added in AIM-V media to a final volume of 150 μl. Measurement was performed 42 hours post addition with the Xcelligence System (Roche).

The results obtained for 200.000 PBMCs (effector cells) freshly isolated from Donor 3 co-cultured with 20.000 adherent Colo38 cells (96 well plates in triplicates) are shown in FIG. 16 (lysis of cells after 42 hours of incubation with multi-function protein). 25% of the PBMCs are CD8-positive T cells of which in turn 3% are CMV-pp65-peptide specific resulting in approx. 1.500 CMV-pp65-peptide-specific CD8+ T cells per 20.000 Colo38 target cells (real E:T (Effector to Target Cell Ratio)=1:13).

Results

The MCSP binding multi-function protein triggers lysis of Colo38 tumor cells through human CMV-specific T-cells.

### Example 11

### LDH Release Assay

ACEA plates were centrifuged for 7 min. at 910 rpm. 50 μl of ACEA supernatants were transferred in another 96well flat bottom plate (Costar). LDH reagent (Cytotoxicity Detection Kit, Roche, Cat.#11644793001) 1 and 2 are diluted according to the manufacturer's instructions and 50 μl of the solution were added to the supernatant. Absorption was detected after an incubation period of 5 to 25 minutes in Tecan Reader Sunrise (Tecan). Total lysis is detected through addition of 1% Triton X-100 (Sigma, Cat.# T-8787) to the target cells before centrifugation.

The results obtained for 200.000 PBMCs (effector cells) freshly isolated from Donor 3 co-cultured with 20.000 adherent Colo38 cells (96 well plates in triplicates) are shown in FIG. 17 (LDH release after 48 hours of incubation with multi-function protein). 200.000 PBMCs freshly isolated from Donor 3 were co-cultured with 20.000 adherent Colo38 cells (96 well plates in triplicates). 25% of the PBMCs are CD8-positive T cells of which in turn 3% are CMV-pp65-peptide specific resulting in approx. 1.500 CMV-pp65-peptide-specific CD8+ T cells per 20.000 Colo38 target cells (E:T (Effector to Target Cell Ratio)=1:13)

Results

The MCSP binding multi-function protein triggers lysis of Colo38 tumor cells through human CMV-specific T-cells.

### Example 12

### Cytotoxicity Assay

PBMCs were obtained from whole blood via Ficoll centrifugation. 1×10⁷ PBMCs per ml were diluted in T-cell medium (RPMI 1640 supplemented with 10% HS, 2 mM glutamine) and peptide exchange on HLA-A0201 molecules was accomplished by addition of 50 μg/ml CMV pp65 peptide to the suspension. After 2-3 h incubation the PBMCs were diluted 1:10 and plated á 200 μl in 96well round bottom plates. On day 3 20 U/ml IL-2, 25 ng/ml IL-7 and IL-15 were added. After 14 d a re-stimulation was performed.

The stimulated T-cells were washed two times in the 96well plates and diluted in 200 μl T-cell medium from which 80 μl were transferred in new 96well round bottom plates.

PBMCs were stimulated according to the protocol above. Stimulated PBMCs were irradiated after peptide exchange with 4000 Gray, washed with T-cell medium twice and 1×10⁵ PBMCs were pipetted to the 80 μl of T-cells. On day 3 20 U/ml IL-2, 25 ng/ml IL-7 and IL-15 were added. An Xcelligence cytotoxicity assay was performed with re-stimulated T-cells on day 11.

63% of the effector cells are CMV-pp65-peptide specific CD8⁺ T-cells.

The target cell to effector cell ratio was 1:3.5. Cell lysis was determined 10 hours after addition of the respective multi-function protein. The multi-function fusion protein was added to a final concentration of 1 μg/ml.

The results are shown in FIG. 18A for Colo38 cells and in FIG. 18B for WM266 cells.

### Example 13

### Disulfide-Stabilized Multi-Function Proteins

A disulfide-bridge between position 11 and 227 of the antigen presenting domain in the multi-function protein as reported herein has been introduced.

The amino acid sequence of the disulfide stabilized antigen presenting domain is:

(SEQ ID NO: 137)
NLVPMVATVGCGGSGGGGSGGGGSIQRTPKIQVYSRHPAENGKSNFLNCY
VSGFHPSDIEVDLLKNGERIEKVEHSDLSFSKDWSFYLLYYTEFTPTEKD
EYACRVNHVTLSQPKIVKWDRDMGGGGSGGGGSGGGGSGGGGSGSHSMRY
FFTSVSRPGRGEPRFIAVGYVDDTQFVRFDSDAASQRMEPRAPWIEQEGP
EYWDGETRKVKAHSQTHRVDLGTLRGCYNQSEAGSHTVQRMYGCDVGSDW
RFLRGYHQYAYDGKDYIALKEDLRSWTAADMAAQTTKHKWEAAHVAEQLR
AYLEGTCVEWLRRYLENGKETLQRTDAPKTHMTHHAVSDHEATLRCWALS
FYPAEITLTWQRDGEDQTQDTELVETRPAGDGTFQKWAAVVVPSGQEQRY
TCHVQHEGLPKPLTLRWGSGQVQLQESGPGLVKPSQTLSLTCTVSGGSIT
SGYYWNWIRQHPGKGLEWIGYITYDGSNNYNPSLKSRVTISRDTSKNQFS
LKLSSVTAADTAVYYCADFDYWGQGTLVTVSSASTKGPSVFPLAPSSKST
SGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAA
GGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALGAPIEKT
ISKAKGQPREPQVCTLPPSRDELTKNQVSLSCAVKGFYPSDIAVEWESNG
QPENNYKTTPPVLDSDGSFFLVSKLTVDKSRWQQGNVFSCSVMHEALHNH
YTQKSLSLSPGK.

Without disulfide stabilization 6.4 mg of multi-function protein can be obtained from 1 l cultivation supernatant after protein A affinity purification. The final yield after size exclusion chromatography to separate aggregates was 2.2 mg.

With disulfide stabilization 17.8 mg of multi-function protein can be obtained from 1 l cultivation supernatant after protein A affinity purification. The final yield after size exclusion chromatography was 11.4 mg due to a lower amount of aggregates due to increased thermal stability by the introduced disulfide bridge.

The analytical size exclusion chromatograms after protein A affinity chromatography but prior to aggregate removal by preparative size exclusion chromatography are shown in FIGS. 19A-1 and 19B-1.

The disulfide-linked multi-function proteins show the same functionality as the non-disulfide-linked multi-function proteins.

PBMCs were obtained from whole blood via Ficoll centrifugation. 1×10⁷ PBMCs per ml were diluted in T-cell medium (RPMI 1640, supplemented with 10% HS, 2 mM glutamine) and peptide exchange on HLA-A0201 molecules was accomplished by addition of 50 μg/ml CMV pp65 peptide to the suspension. After 2-3 h incubation the PBMCs are diluted 1:10 and plated á 200 μl in 96well round bottom plates. On day 3 20 U/ml IL-2, 25 ng/ml IL-7 and IL-15 were added. Cells were taken 11 d after primary stimulation; 45% of the cells were CMV specific. The results for a target cell to effector cell ration of 1:3 are shown in FIG. 20.

1. A multi-function protein, characterized in that it comprises
exactly one antigen presenting domain,
exactly one antibody Fc-region, and
at least one antigen binding site,
wherein the antigen presenting domain comprises in N- to C-terminal direction
either
(i) a T-cell response eliciting peptide,
(ii) a β2-microglobulin, and
(iii) the extracellular domains α1, α2, and α3 of a class I MHC molecule with a relative frequency of 1% or more,
or
(i) a T-cell response eliciting peptide,
(ii) the extracellular domains α1, α2, and α3 of a class I MHC molecule with a relative frequency of 1% or more, and
(iii) a β2-microglobulin,
wherein the antigen binding site binds to a melanoma-associated chondroitin sulfate proteoglycan (MCSP).

2. The multi-function protein according to claim 1, characterized in that the antibody Fc-region comprises a first and second disulfide-linked Fe-region polypeptide, whereby the antigen binding site comprises the first Fc-region polypeptide.

3. The multi-function protein according to claim 1, characterized in that the antigen binding site comprises i) a pair of an antibody heavy chain and an antibody light chain, or ii) a scFv fusion polypeptide comprising in N- to C-terminal direction a scFv antibody fragment and an antibody Fc-region polypeptide, or iii) a scFab fusion polypeptide comprising in N- to C-terminal direction a scFab and an antibody Fc-region polypeptide.

4. The multi-function protein according to claim 1, characterized in that i) the antigen presenting domain is linked to the N-terminus of the heavy chain or to the N-terminus of the light chain of the antigen binding site, or ii) the antigen presenting domain is linked to the C-terminus of the heavy chain or to the C-terminus of the light chain of the antigen binding site, or iii) the antigen presenting domain is linked to the N- or C-terminus of the scFv fusion polypeptide, or iv) the antigen presenting domain is linked to the N- or C-terminus of the scFab fusion polypeptide, or iv) the antigen presenting domain is linked to the N- or C-terminus of the second Fc-region polypeptide.

5. (canceled)

6. The multi-function protein according to claim 1, characterized in that the T-cell response eliciting peptide is a virus-derived peptide.

7. The multi-function protein according to claim 6, characterized in that the virus-derived peptide is a human cytomegalovirus-derived peptide.

8. The multi-function protein according to claim 1, characterized in that the virus-derived peptide has the amino acid sequence of SEQ ID NO: 01.

9. The multi-function protein according to claim 1, characterized in that the class I MHC molecule with a relative frequency of 1% or more is selected from the group comprising HLA-A*0201, HLA-A*1101, HLA-A*2402, HLA-A*340101, HLA-C*0304, HLA-C*0401, and HLA-C*0702.

10. The multi-function protein according to claim 1, characterized in that the class I MHC molecule with a relative frequency of less than 1% is selected from the group comprising HLA-B*4201, HLA-B*5901, HLA-B*6701, and HLA-B*7802.

11. The multi-function protein according to claim 1, characterized in that the antigen presenting domain comprises
(i) a virus-derived peptide,
(ii) β2-microglobulin, and
(iii) the soluble HLA-A allele A*0201.

12. The multi-function protein according to claim 1, characterized in that the antigen presenting domain comprises in N- to C-terminal direction:
(i) a virus-derived peptide that has an amino acid sequence selected from the group comprising SEQ ID NO: 01 to SEQ ID NO: 70,
(ii) a first linker peptide that has an amino acid sequence selected from the group comprising SEQ ID NO: 77, 78, 79, 82, 83, and 84,
(iii) a β2-microglobulin that has an amino acid sequence of SEQ ID NO: 71,
(iv) a second linker peptide that has an amino acid sequence selected from the group comprising SEQ ID NO: 77, 78, 79, 82, 83, and 84,
(v) the extracellular domains α1, α2, and α3 of a class I MHC molecule that has an amino acid sequence of SEQ ID NO: 72, and
(vi) a third linker peptide that has an amino acid sequence selected from the group comprising SEQ ID NO: 73, 77, 78, 79, 82, 83, 84, and 136.

13. A pharmaceutical formulation comprising the multi-function protein according to claim 1 and optionally a pharmaceutically acceptable carrier.

14. (canceled)

15. (canceled)

16. (canceled)

17. (canceled)

18. A method of treating cancer comprising administering to a subject in need thereof the pharmaceutical formulation according to claim 13.

19. A method of attracting virus-specific cytotoxic T-cells to a target comprising administering to a subject in need thereof the multi-specific protein according to claim 1.

20. A method of removing cancer cells or virus infected cells comprising administering to a subject in need thereof the multi-specific protein according to claim 1.


SEQUENCE LISTING (138 sequences)

SEQ ID NO: 1 - protein, 9 aa, Human cytomegalovirus
NLVPMVATV

SEQ ID NO: 2 - protein, 9 aa, Human cytomegalovirus
VTEHDTLLY

SEQ ID NO: 3 - protein, 9 aa, Human cytomegalovirus
NTDFRVLEL

SEQ ID NO: 4 - protein, 9 aa, Human cytomegalovirus
CVETMCNEY

SEQ ID NO: 5 - protein, 9 aa, Human cytomegalovirus
VLEETSVML

SEQ ID NO: 6 - protein, 9 aa, Human cytomegalovirus
NLVPMVATV

SEQ ID NO: 7 - protein, 9 aa, Human cytomegalovirus
RIFAELEGV

SEQ ID NO: 8 - protein, 9 aa, Human cytomegalovirus
IIYTRNHEV

SEQ ID NO: 9 - protein, 9 aa, Human cytomegalovirus
VLAELVKQI

SEQ ID NO: 10 - protein, 9 aa, Human cytomegalovirus
AVGGAVASV

SEQ ID NO: 11 - protein, 9 aa, Human cytomegalovirus
TVRSHCVSK

SEQ ID NO: 12 - protein, 9 aa, Human cytomegalovirus
IMREFNSYK

SEQ ID NO: 13 - protein, 10 aa, Human cytomegalovirus
GPISHGHVLK

SEQ ID NO: 14 - protein, 9 aa, Human cytomegalovirus
ATVQGQNLK

SEQ ID NO: 15 - protein, 9 aa, Human cytomegalovirus
VYALPLKML

SEQ ID NO: 16 - protein, 10 aa, Human cytomegalovirus
AYAQKIFKIL

SEQ ID NO: 17 - protein, 9 aa, Human cytomegalovirus
QYDPVAALF

SEQ ID NO: 18 - protein, 9 aa, Human cytomegalovirus
YVKVYLESF

SEQ ID NO: 19 - protein, 9 aa, Human cytomegalovirus
DIYRIFAEL

SEQ ID NO: 20 - protein, 10 aa, Human cytomegalovirus
VFETSGGLVV

SEQ ID NO: 21 - protein, 9 aa, Human cytomegalovirus
KARDHLAVL

SEQ ID NO: 22 - protein, 9 aa, Human cytomegalovirus
QARLTVSGL

SEQ ID NO: 23 - protein, 9 aa, Human cytomegalovirus
KARAKKDEL

SEQ ID NO: 24 - protein, 9 aa, Human cytomegalovirus
QIKVRVDMV

SEQ ID NO: 25 - protein, 9 aa, Human cytomegalovirus
RRRHRQDAL

SEQ ID NO: 26 - protein, 9 aa, Human cytomegalovirus
ARVYEIKCR

SEQ ID NO: 27 - protein, 9 aa, Human cytomegalovirus
KMQVIGDQY

SEQ ID NO: 28 - protein, 9 aa, Human cytomegalovirus
NVRRSWEEL

SEQ ID NO: 29 - protein, 12 aa, Human cytomegalovirus
CPSQEPMSIYVY

SEQ ID NO: 30 - protein, 13 aa, Human cytomegalovirus
KPGKISHIMLDVA

SEQ ID NO: 31 - protein, 9 aa, Human cytomegalovirus
ELRRKMMYM

SEQ ID NO: 32 - protein, 9 aa, Human cytomegalovirus
IPSINVHHY

SEQ ID NO: 33 - protein, 9 aa, Human cytomegalovirus
FEQPTETPP

SEQ ID NO: 34 - protein, 9 aa, Human cytomegalovirus
YAYIYTTYL

SEQ ID NO: 35 - protein, 11 aa, Human cytomegalovirus
QEFFWDANDIY

SEQ ID NO: 36 - protein, 9 aa, Human cytomegalovirus
YEQHKITSY

SEQ ID NO: 37 - protein, 9 aa, Human cytomegalovirus
QEPMSIYVY

SEQ ID NO: 38 - protein, 10 aa, Human cytomegalovirus
SEHPTFTSQY

SEQ ID NO: 39 - protein, 9 aa, Human cytomegalovirus
QAIRETVEL

SEQ ID NO: 40 - protein, 9 aa, Human cytomegalovirus
TRATKMQVI

SEQ ID NO: 41 - protein, 8 aa, Human cytomegalovirus
DALPGPCI

SEQ ID NO: 42 - protein, 9 aa, Human cytomegalovirus
CEDVPSGKL

SEQ ID NO: 43 - protein, 9 aa, Human cytomegalovirus
HERNGFTVL

SEQ ID NO: 44 - protein, 13 aa, Human cytomegalovirus
PTFTSQYRIQGKL

SEQ ID NO: 45 - protein, 9 aa, Human cytomegalovirus
QMWQARLTV

SEQ ID NO: 46 - protein, 12 aa, Human cytomegalovirus
HELLVLVKKAQL

SEQ ID NO: 47 - protein, 12 aa, Human cytomegalovirus
DDYSNTHSTRYV

SEQ ID NO: 48 - protein, 9 aa, Human immunodeficiency virus
SLYNTVATL

SEQ ID NO: 49 - protein, 9 aa, Human herpesvirus 4
GLCTLVAML

SEQ ID NO: 50 - protein, 9 aa, Influenza A virus
GILGFVFTL

SEQ ID NO: 51 - protein, 9 aa, Hepatitis B virus
STNRQSGRQ

SEQ ID NO: 52 - protein, 9 aa, Human T-cell lymphotropic virus type 1
LLFGYPVYV

SEQ ID NO: 53 - protein, 9 aa, Artificial Sequence
  V-jun Sarcoma Virus 17 Oncogene Homolog (JUN)
FAEGFVRAL

SEQ ID NO: 54 - protein, 9 aa, Human adenovirus type 3
LIVIGILIL

SEQ ID NO: 55 - protein, 8 aa, Hepatitis C virus
ILHTPGCV

SEQ ID NO: 56 - protein, 9 aa, Dengue virus
WYAQIQPHW

SEQ ID NO: 57 - protein, 9 aa, Dengue virus
AFSGVSWTM

SEQ ID NO: 58 - protein, 9 aa, Dengue virus
ILIGVVITW

SEQ ID NO: 59 - protein, 9 aa, Dengue virus
MMIPTVVAF

SEQ ID NO: 60 - protein, 9 aa, Dengue virus
PFPQSNAPI

SEQ ID NO: 61 - protein, 9 aa, Dengue virus
LLLTLLATV

SEQ ID NO: 62 - protein, 9 aa, Dengue virus
IVLEHGSCV

SEQ ID NO: 63 - protein, 9 aa, Dengue virus
LLFKTENGV

SEQ ID NO: 64 - protein, 9 aa, Dengue virus
PLNEAIMAV

SEQ ID NO: 65 - protein, 9 aa, Dengue virus
NLVRLQSGV

SEQ ID NO: 66 - protein, 9 aa, Dengue virus
LVISGLFPV

SEQ ID NO: 67 - protein, 9 aa, Dengue virus
LLLVAHYAI

SEQ ID NO: 68 - protein, 9 aa, Dengue virus
LALLAAFKV

SEQ ID NO: 69 - protein, 9 aa, Dengue virus
VILAGPMPV

SEQ ID NO: 70 - protein, 9 aa, Dengue virus
HVLGRLITV

SEQ ID NO: 71 - protein, 99 aa, Homo sapiens [sequence omitted]

SEQ ID NO: 72 - protein, 274 aa, Homo sapiens [sequence omitted]

SEQ ID NO: 73 - protein, 2 aa, Artificial Sequence
  linker peptide 1
GS

SEQ ID NO: 74 - protein, 3 aa, Artificial Sequence
  linker peptide 2
GGS

SEQ ID NO: 75 - protein, 4 aa, Artificial Sequence
  linker peptide 3
GGGS

SEQ ID NO: 76 - protein, 8 aa, Artificial Sequence
  linker peptide 4
GGGSGGGS

SEQ ID NO: 77 - protein, 12 aa, Artificial Sequence
  linker peptide 5
GGGSGGGSGGGS

SEQ ID NO: 78 - protein, 16 aa, Artificial Sequence
  linker peptide 6
GGGSGGGSGGGSGGGS

SEQ ID NO: 79 - protein, 20 aa, Artificial Sequence
  linker peptide 7
GGGSGGGSGGGSGGGSGGGS

SEQ ID NO: 80 - protein, 5 aa, Artificial Sequence
  linker peptide 8
GGGGS

SEQ ID NO: 81 - protein, 10 aa, Artificial Sequence
  linker peptide 9
GGGGSGGGGS

SEQ ID NO: 82 - protein, 15 aa, Artificial Sequence
  linker peptide 10
GGGGSGGGGSGGGGS

SEQ ID NO: 83 - protein, 20 aa, Artificial Sequence
  linker peptide 11
GGGGSGGGGSGGGGSGGGGS

SEQ ID NO: 84 - protein, 25 aa, Artificial Sequence
  linker peptide 12
GGGGSGGGGSGGGGSGGGGSGGGGS

SEQ ID NO: 85 - protein, 107 aa, Homo sapiens
APELLGGPSVFLFPPKPKDTLMISRTPEVTCVWDVSHEDPEVKFNWYVDGVEVHNAKTKPREEQESTYRWSVLTVLHQDWLNGKEYKCKVSNKALPAPIEKTISKAK

SEQ ID NO: 86 - protein, 106 aa, Homo sapiens [sequence omitted]

SEQ ID NO: 87 - protein, 232 aa, Homo sapiens
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 232 aa, Artificial Sequence
  variant human Fc-region of the IgG1 isotype with the mutations L234A, L235A
EPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 232 aa, Artificial Sequence
  variant human Fc-region of the IgG1 isotype with a hole mutation
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 90 - protein, 232 aa, Artificial Sequence
  variant human Fc-region of the IgG1 isotype with a knob mutation
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 232 aa, Artificial Sequence
  variant human Fc-region of the IgG1 isotype with a L234A, L235A and hole mutation
EPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 92 - protein, 232 aa, Artificial Sequence
  variant human Fc-region of the IgG1 isotype with a L234A, L235A and knob mutation
EPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 232 aa, Artificial Sequence
  variant human Fc-region of the IgG1 isotype with a P329G mutation
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 232 aa, Artificial Sequence
  variant human Fc-region of the IgG1 isotype with a L234A, L235A and P329G mutation
EPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 232 aa, Artificial Sequence
  variant human Fc-region of the IgG1 isotype with a P239G and hole mutation
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 96 - protein, 232 aa, Artificial Sequence
  variant human Fc-region of the IgG1 isotype with a P329G and knob mutation
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 232 aa, Artificial Sequence
  variant human Fc-region of the IgG1 isotype with a L234A, L235A, P329G and hole mutation
EPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 98 - protein, 232 aa, Artificial Sequence
  variant human Fc-region of the IgG1 isotype with a L234A, L235A, P329G and knob mutation
EPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - protein, 229 aa, Artificial Sequence
  variant human Fc-region of the IgG4 isotype with a S228P and L235E mutation
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 101 - protein, 229 aa, Artificial Sequence
  variant human Fc-region of the IgG4 isotype with a S228P, L235E and P329G mutation
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - protein, 229 aa, Artificial Sequence
  variant human Fc-region of the IgG4 isotype with a S228P and L235E mutation
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 103 - protein, 229 aa, Artificial Sequence
  variant human Fc-region of the IgG4 isotype with a S228P, L235E and P329G mutation
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 104 - protein, 11 aa, Artificial Sequence
  HVR-L1
SASQGIRNYLN

SEQ ID NO: 105 - protein, 7 aa, Artificial Sequence
  HVR-L2
YTSSLHS

SEQ ID NO: 106 - protein, 9 aa, Artificial Sequence
  HVR-L3
QQYSKLPWT

SEQ ID NO: 107 - protein, 11 aa, Artificial Sequence
  HVR-L1
RASQGIRNYLN

SEQ ID NO: 108 - protein, 11 aa, Artificial Sequence
  HVR-H1
GYSITSGYYWN

SEQ ID NO: 109 - protein, 16 aa, Artificial Sequence
  HVR-H2
YITYDGSNNYNPSLKN

SEQ ID NO: 110 - protein, 3 aa, Artificial Sequence
  HVR-H3
FDY

SEQ ID NO: 111 - protein, 11 aa, Artificial Sequence
  HVR-H1
GGSITSGYYWN

SEQ ID NO: 112 - protein, 16 aa, Artificial Sequence
  HVR-H2
YITYDGSNNYNPSLKS

SEQ ID NO: 113 - protein, 107 aa, Artificial Sequence
  LC007 Chimeric Antibody VL
DIVLTQSPSSLSASLGDRVTISCSASQGIRNYLNWYQQRPDGTVKLLIYYTSSLHSGVPSRFSGSGSGTDYSLTISNLEPEDIATYYCQQYSKLPWTFGGGTKLEIK

SEQ ID NO: 114 - protein, 112 aa, Artificial Sequence
  LC007 Chimeric Antibody VH
EVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSS

SEQ ID NO: 115 - protein, 107 aa, Artificial Sequence
  LC007 Humanized Antibody ML2 VL
DIQMTQSPSSLSASVGDRVTITCRASQGIRNYLNWYQQKPGKAPKLLIYYTSSLHSGVPSRFSGSGSGTDFTLTISSLQPEDFATYYCQQYSKLPWTFGQGTKVEIK

SEQ ID NO: 116 - protein, 112 aa, Artificial Sequence
  LC007 Humanized Antibody M4-3 VH
QVQLQESGPGLVKPSQTLSLTCTVSGGSITSGYYWNWIRQHPGKGLEWIGYITYDGSNNYNPSLKSRVTISRDTSKNQFSLKLSSVTAADTAVYYCADFDYWGQGTLVTVSS

SEQ ID NO: 117 - protein, 862 aa, Artificial Sequence
  MHC-I-VH (MCSP)-IgG1 Fc-region L234A, L235A, P329G, T366S, L368A, Y407V mutant amino acid sequence
NLVPMVATVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWGSGQVQLQESGPGLVKPSQTLSLTCTVSGGSITSGYYWNWIRQHPGKGLEWIGYITYDGSNNYNPSLKSRVTISRDTSKNQFSLKLSSVTAADTAVYYCADFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 118 - protein, 442 aa, Artificial Sequence
  VH(MCSP)-IgG1 Fc-region L234A, L235A, P329G, T366W mutant amino acid sequence
QVQLQESGPGLVKPSQTLSLTCTVSGGSITSGYYWNWIRQHPGKGLEWIGYITYDGSNNYNPSLKSRVTISRDTSKNQFSLKLSSVTAADTAVYYCADFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 214 aa, Artificial Sequence
  VL(MCSP)-CL amino acid sequence
DIQMTQSPSSLSASVGDRVTITCRASQGIRNYLNWYQQKPGKAPKLLIYYTSSLHSGVPSRFSGSGSGTDFTLTISSLQPEDFATYYCQQYSKLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 215 aa, Artificial Sequence
  Ig Light Chain
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 448 aa, Artificial Sequence
  Ig Heavy Chain (IgG1-L234A, L235A mutant)
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 448 aa, Artificial Sequence
  Ig Heavy Chain (IgG1-L234A, L235A mutant with knob variation)
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 448 aa, Artificial Sequence
  Ig Heavy Chain (IgG1-L234A, L235A mutant with hole variation)
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 124 - protein, 232 aa, Artificial Sequence; identical to SEQ ID NO: 92
  Ig heavy chain Fc region (IgG1-L234A, L235A mutant Fc-region)

SEQ ID NO: 125 - protein, 246 aa, Artificial Sequence
  scFv
QVELVESGGGVVQPGRSQRLSCAASGFTFSSYGMHWVRQAPGKCLEWVAIIWFDGSSTYYADSVRGRFTISRDNSKNTLYLQMNSLRAEDTAVYFCARELGRRYFDLWGRGTLVSVSSGGGGSGGGGSGGGGSGGGGSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGCGTKVESK

SEQ ID NO: 126 - protein, 214 aa, Artificial Sequence
  Murine anti-MCSP monoclonal light chain antibody amino acid sequence (kappa)
DIVLTQSPSSLSASLGDRVTISCSASQGIRNYLNWYQQRPDGTVKLLIYYTSSLHSGVPSRFSGSGSGTDYSLTISNLEPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 214 aa, Artificial Sequence; identical to SEQ ID NO: 119
  Humanized anti-MCSP monoclonal light chain antibody amino acid sequence (kappa)

SEQ ID NO: 128 - protein, 442 aa, Artificial Sequence
  Murine anti-MCSP monoclonal heavy chain antibody amino acid sequence (IgG1 L234A, L235A mutant)
EVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 442 aa, Artificial Sequence
  Humanized anti-MCSP monoclonal heavy chain antibody amino acid sequence (IgG1 L234A, L235A mutant)
QVQLQESGPGLVKPSQTLSLTCTVSGGSITSGYYWNWIRQHPGKGLEWIGYITYDGSNNYNPSLKSRVTISRDTSKNQFSLKLSSVTAADTAVYYCADFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 442 aa, Artificial Sequence
  Murine anti-MCSP monoclonal heavy chain antibody amino acid sequence (IgG1 L234A, L235A mutant and knob variant)
EVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 442 aa, Artificial Sequence
  Humanized anti-MCSP monoclonal heavy chain antibody amino acid sequence (IgG1 L234A, L235A mutant and knob variant)
QVQLQESGPGLVKPSQTLSLTCTVSGGSITSGYYWNWIRQHPGKGLEWIGYITYDGSNNYNPSLKSRVTISRDTSKNQFSLKLSSVTAADTAVYYCADFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 442 aa, Artificial Sequence
  Murine anti-MCSP monoclonal heavy chain antibody amino acid sequence (IgG1 L234A, L235A mutant and hole variant)
EVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 133 - protein, 442 aa, Artificial Sequence
  Humanized anti-MCSP monoclonal heavy chain antibody amino acid sequence (IgG1 L234A, L235A mutant and hole variant)
QVQLQESGPGLVKPSQTLSLTCTVSGGSITSGYYWNWIRQHPGKGLEWIGYITYDGSNNYNPSLKSRVTISRDTSKNQFSLKLSSVTAADTAVYYCADFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 134 - protein, 239 aa, Artificial Sequence
  Disulfide-stabilized single chain Fv of murine anti-MCSP monoclonal antibody
EVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSGGGGSGGGGSGGGGSGGGGSDIVLTQSPSSLSASLGDRVTISCSASQGIRNYLNWYQQRPDGTVKLLIYYTSSLHSGVPSRFSGSGSGTDYSLTISNLEPEDIATYYCQQYSKLPWTFGGGTKLEIK

SEQ ID NO: 135 - protein, 239 aa, Artificial Sequence
  Disulfide-stabilized single chain Fv of humanized anti-MCSP monoclonal antibody
QVQLQESGPGLVKPSQTLSLTCTVSGGSITSGYYWNWIRQHPGKGLEWIGYITYDGSNNYNPSLKSRVTISRDTSKNQFSLKLSSVTAADTAVYYCADFDYWGQGTLVTVSSGGGGSGGGGSGGGGSGGGGSDIQMTQSPSSLSASVGDRVTITCRASQGIRNYLNWYQQKPGKAPKLLIYYTSSLHSGVPSRFSGSGSGTDFTLTISSLQPEDFATYYCQQYSKLPWTFGQGTKVEIK

SEQ ID NO: 136 - protein, 3 aa, Artificial Sequence
  linker peptide 13
GSG

SEQ ID NO: 137 - protein, 862 aa, Artificial Sequence [sequence omitted]

SEQ ID NO: 138 - protein, 2322 aa, Homo sapiens
MQSGPRPPLPAPGLALALTLTMLARLASAASFFGENHLEVPVATALTDIDLQLQFSTSQPEALLLLAAGPADHLLLQLYSGRLQVRLVLGQEELRLQTPAETLLSDSIPHTVVLTVVEGWATLSVDGFLNASSAVPGAPLEVPYGLFVGGTGTLGLPYLRGTSRPLRGCLHAATLNGRSLLRPLTPDVHEGCAEEFSASDDVALGFSGPHSLAAFPAWGTQDEGTLEFTLTTQSRQAPLAFQAGGRRGDFIYVDIFEGHLRAVVEKGQGTVLLHNSVPVADGQPHEVSVHINAHRLEISVDQYPTHTSNRGVLSYLEPRGSLLLGGLDAEASRHLQEHRLGLTPEATNASLLGCMEDLSVNGQRRGLREALLTRNMAAGCRLEEEEYEDDAYGHYEAFSTLAPEAWPAMELPEPCVPEPGLPPVFANFTQLLTISPLVVAEGGTAWLEWRHVQPTLDLMEAELRKSQVLFSVTRGARHGELELDIPGAQARKMFTLLDVVNRKARFIHDGSEDTSDQLVLEVSVTARVPMPSCLRRGQTYLLPIQVNPVNDPPHIIFPHGSLMVILEHTQKPLGPEVFQAYDPDSACEGLTFQVLGTSSGLPVERRDQPGEPATEFSCRELEAGSLVYVHRGGPAQDLTFRVSDGLQASPPATLKVVAIRPAIQIHRSTGLRLAQGSAMPILPANLSVETNAVGQDVSVLFRVTGALQFGELQKQGAGGVEGAEWWATQAFHQRDVEQGRVRYLSTDPQHHAYDTVENLALEVQVGQEILSNLSFPVTIQRATVWMLRLEPLHTQNTQQETLTTAHLEATLEEAGPSPPTFHYEVVQAPRKGNLQLQGTRLSDGQGFTQDDIQAGRVTYGATARASEAVEDTFRFRVTAPPYFSPLYTFPIHIGGDPDAPVLTNVLLVVPEGGEGVLSADHLFVKSLNSASYLYEVMERPRHGRLAWRGTQDKTTMVTSFTNEDLLRGRLVYQHDDSETTEDDIPFVATRQGESSGDMAWEEVRGVFRVAIQPVNDHAPVQTISRIFHVARGGRRLLTTDDVAFSDADSGFADAQLVLTRKDLLFGSIVAVDEPTRPIYRFTQEDLRKRRVLFVHSGADRGWIQLQVSDGQHQATALLEVQASEPYLRVANGSSLVVPQGGQGTIDTAVLHLDTNLDIRSGDEVHYHVTAGPRWGQLVRAGQPATAFSQQDLLDGAVLYSHNGSLSPRDTMAFSVEAGPVHTDATLQVTIALEGPLAPLKLVRHKKIYVFQGEAAEIRRDQLEAAQEAVPPADIVFSVKSPPSAGYLVMVSRGALADEPPSLDPVQSFSQEAVDTGRVLYLHSRPEAWSDAFSLDVASGLGAPLEGVLVELEVLPAAIPLEAQNFSVPEGGSLTLAPPLLRVSGPYFPTLLGLSLQVLEPPQHGALQKEDGPQARTLSAFSWRMVEEQLIRYVHDGSETLTDSFVLMANASEMDRQSHPVAFTVTVLPVNDQPPILTTNTGLQMWEGATAPIPAEALRSTDGDSGSEDLVYTIEQPSNGRVVLRGAPGTEVRSFTQAQLDGGLVLFSHRGTLDGGFRFRLSDGEHTSPGHFFRVTAQKQVLLSLKGSQTLTVCPGSVQPLSSQTLRASSSAGTDPQLLLYRVVRGPQLGRLFHAQQDSTGEALVNFTQAEVYAGNILYEHEMPPEPFWEAHDTLELQLSSPPARDVAATLAVAVSFEAACPQRPSHLWKNKGLWVPEGQRARITVAALDASNLLASVPSPQRSEHDVLFQVTQFPSRGQLLVSEEPLHAGQPHFLQSQLAAGQLVYAHGGGGTQQDGFHFRAHLQGPAGASVAGPQTSEAFAITVRDVNERPPQPQASVPLRLTRGSRAPISRAQLSVVDPDSAPGEIEYEVQRAPHNGFLSLVGGGLGPVTRFTQADVDSGRLAFVANGSSVAGIFQLSMSDGASPPLPMSLAVDILPSAIEVQLRAPLEVPQALGRSSLSQQQLRVVSDREEPEAAYRLIQGPQYGHLLVGGRPTSAFSQFQIDQGEVVFAFTNFSSSHDHFRVLALARGVNASAVVNVTVRALLHVWAGGPWPQGATLRLDPTVLDAGELANRTGSVPRFRLLEGPRHGRVVRVPRARTEPGGSQLVEQFTQQDLEDGRLGLEVGRPEGRAPGPAGDSLTLELWAQGVPPAVASLDFATEPYNAARPYSVALLSVPEAARTEAGKPESSTPTGEPGPMASSPEPAVAKGGFLSFLEANMFSVIIPMCLVLLLLALILPLLFYLRKRNKTGKHDVQVLTAKPRNGLAGDTETFRKVEPGQAIPLTAVPGQGPPPGGQPDPELLQFCRTPNPALKNGQYWV